(19) United States
(12) Patent Application Publication
(10) Pub. No.: US 2018/0267024 A1
(43) Pub. Date: Sep. 20, 2018

(54) COMPOSITION FOR DETERMINATION OF CELL-MEDIATED IMMUNE RESPONSIVENESS

(71) Applicant: Lophius Biosciences GmbH, Regensburg (DE)

(72) Inventors: Ludwig DEML, Regenstauf (DE); Sascha BARABAS, Bad Abbach (DE); Mathias SCHEMMERER, Landshut (DE)

(21) Appl. No.: 15/577,935

(22) PCT Filed: Jun. 8, 2016
(86) PCT No.: PCT/EP2016/063066

§ 371 Date: Nov. 29, 2017

(30) Foreign Application Priority Data
Jun. 8, 2015 (EP) 15171074.6

(51) Int. Cl. G01N 33/50

(52) U.S. Cl. CPC G01N 33/5091; G01N 2800/24; G01N 33/5047; G01N 33/505

(57) ABSTRACT

The present invention relates to a composition comprising (i) a first substance which is capable to stimulate T cells, (ii) a second substance which is capable to stimulate NK cells (natural killer cells), and (iii) lipopolysaccharide (LPS) and wherein the second substance is a double stranded nucleic acid, single stranded nucleic acid, unmethylated CpG oligodeoxynucleotide, TLR agonist except lipopolysaccharide (LPS), arabinoxylan (BioBran® MGN-3), an immunoglobulin, a murine cytomegalovirus (MCMV)-encoded protein, CCL5 (chemokine (C—C motif) ligand 5), a UL-16-binding protein (ULBP), CD48, CD70, CD155, CD112, Necl-1, B7-H6, ICAM-1, RAE-1 (retinoic acid early inducible 1), H60, Mult1 and/or hemagglutinin, to a method for measuring, determining and/or detecting the status of cell-mediated immune responsiveness of a subject, to a kit comprising the composition according to the invention, to the use of the composition for measuring, determining and/or detecting of cell-mediated immunity (CMI) and/or for detecting, diagnosing, monitoring an immunosuppression condition in a subject.

The present invention relates to a composition comprising (i) a first substance which is capable to stimulate T cells, (ii) a second substance which is capable to stimulate NK cells (natural killer cells), and (iii) lipopolysaccharide (LPS) and wherein the second substance is a double stranded nucleic acid, single stranded nucleic acid, unmethylated CpG oligodeoxynucleotide, TLR agonist except lipopolysaccharide (LPS), arabinoxylan (BioBran® MGN-3), an immunoglobulin, a murine cytomegalovirus (MCMV)-encoded protein, CCL5 (chemokine (C—C motif) ligand 5), a UL-16-binding protein (ULBP), CD48, CD70, CD155, CD112, Necl-1, B7-H6, ICAM-1, RAE-1 (retinoic acid early inducible 1), H60, Multi and/or hemagglutinin, to a method for measuring, determining and/or detecting the status of cell-mediated immune responsiveness of a subject, to a kit comprising the composition according to the invention, to the use of the composition for measuring, determining and/or detecting of cell-mediated immunity (CMI) and/or for detecting, diagnosing, monitoring an immunosuppression condition in a subject.

The human immune system protects the human organism against pathogens or harmful substances, wherein the immune system enables the recognition, combating and rejection of infections and tumors as well as foreign tissue, the memory of information and regulation of itself to prevent over reactions. In immune defense beside the humoral components (e.g. B cells and antibodies) cell mediated immune response plays a crucial role. The cell mediated immune response is divided in an innate and adaptive part.

The innate immune defense is the first-line of defense against invading microbial pathogens. Innate immune response is fast and effective but not pathogen specific and it does not confer long-lasting or protective immunity. Natural killer cells (NK cells) form here one of the first lines of defense against pathogens and tumors.

On all nucleated cells MHC class I molecules are expressed presenting endogenous peptides. In case of an infection also cell foreign epitopes of the pathogen are presented. These are recognized by cytotoxic T cells (CTL), whereupon the cell is eliminated. Tumors or viral infections can result in a suppression of the expression and surface exposure of MHC class I molecules, whereby these cells are no longer effectively recognized and eliminated by CTL. NK cells may recognize the decreased expression of MHC class I molecules on infected cells by receptors such as the killer immunoglobulin-like receptor (killer cell immunoglobulin-like receptor, KIR) and eliminate these cells by release of perforins and granzymes. The function and mode of action of NK cells are described in detail in the review Vivier et al., 2008, Nature Immunology, Vol. 9, No. 5.

In addition to this defense function foreign structures, so-called pathogen-associated molecular patterns (pathogen-associated molecular patterns, PAMPs) are detected by structure-recognizing receptors (pattern recognition receptors, PRR). So far, three classes of PRR are described, the RIG-I-like receptors (RIG-I-like receptor, DFR), Toll-like receptors (toll-like receptor, TLR) and NOD-like receptors (NOD-like receptor, NLR). The so far discovered 10 human Toll-like receptors (TLR1 to 10) recognize structures such as fatty acids (TLR2), double-stranded RNA (TLR3), lipopolysaccharide (TLR4), flagellin (TLR5), Imidazoquinolin (TLR7), viral single-stranded RNA (TLR8) and viral DNA (TLR9) (Sandor & Buc, 2005). NK cells have endosomal Toll-like receptors TLR3, as well as the cytoplasmic RIG-I-like receptors RIG-I and MDA-5 and thus may detect viral dsRNA. This leads to the activation of the transcription factor IRF3 (interferon regulatory factor 3), followed by the production and secretion of type I interferons and inflammatory cytokines (Fredericksen et al., 2008).

Antigen-presenting cells build the connection between the innate and adative part of the immune system. These cells represent cell's own—and in case of infection foreign peptides on MHC class I and II molecules to T cells of the adaptive immune response and lipids via the MHC-like molecule CD1d to NKT cells of the innate immune response. Thus, APC help to identify infections and to initiate the adaptive immune response.

The presentation of peptides via MHC class II molecule takes place via the so-called professional APC. These include dendritic cells (DC), monocytes, macrophages and certain B lymphocytes and certain activated epithelial cells, which are able to actively engage foreign structures by endocytosis, phagocytosis or pinocytosis and to degrade intracellularly. The endosome with the remaining heterologous peptide rests are then fused to the MHC class II molecules loaded endosome. There, the peptides may, depending on their affinity bind to MHC class II molecules whereupon the complex migrates to the cell surface and presents the peptide to T helper cells. The exogenous peptides presented on the MHC class II molecule are usually between 13 to 21 amino acids long, although also longer polypeptides as well as whole proteins may be presented.

Another way of peptide presentation is via the MHC class I molecule. This molecule is found in all nucleated cells and presents peptides, ranging from 8 to 11 amino acids in length, which are typically derived from protein antigens in the cytosol that arise from conventional as well as cryptic translational reading frames. The presentation of heterologous peptides occurs, for example, after an infection of the cell with a pathogen. Cytosolic pathogen-derived antigens are degraded by the proteasome into peptides, which are then transported into the endoplasmic reticulum and bound depending on their affinity to MHC class I molecules. If a peptide binds to an MHC class I molecule, then this complex moves to the cell surface, in order to present the peptide to cytotoxic T cells (CTL). It is now known that exogenous proteins get in these endogenous processing pathway and can lead to peptide presentation on MHC-I molecules. In vivo cross-presentation is mainly carried out by specific dendritic cell (DC) subsets through an adaptation of their endocytic and phagocytic pathways. In vitro, cross-presentation of several exogenous model antigens is achieved by several types of APC such as dendritic cells and macrophages, but also by B cells, endothelial cells and neutrophils via various routes for antigen delivery from endosomes and phagosomes to the cytosol or by direct processing of captured antigens in intracellular vesicles or at the plasma membrane (Joffre et al. 2012, Nature Reviews Immunology, 12, 557-569).

Both endogenous and exogenous lipids are presented on the MHC-like molecule CD1d which is expressed on thymocytes, B cells, monocytes, in vivo activated T cells and is weakly expressed on resting T cells (Exley et al., 2000). The endosomal lipid loading of CD1d is supported by cofactors which extract the lipids from lipid bilayers and bridge the lipid-water interface (Darmoise et al., 2010).

Pathogen or disease-specific T helper cells (Th-cells) and cytotoxic T cells (CTL) are specifically activated by using heterologous peptide-loaded APC and then contribute to combat an infection via various effector mechanisms. In addition to heterologous peptides also foreign lipids are presented which are detected by natural killer T cells (NKT cells).

T helper cells are important for the coordination of the immune response. They play for example, an important role in the stimulation of B-cells for production of specific antibodies, and activation of cytotoxic T-cells. They recognize exogenous peptides presented on MHC class II molecules by using their T-cell receptor (TCR). The glycoprotein CD4 of the Th cells acts as a coreceptor of the TCR and enhances his signal. If a pathogenic epitope is detected, a specific Defense reaction occurs depending on the type of Th-cell. Meanwhile, many different types of Th cells were identified like Th1, Th2 and Th17. Th1 cells produce besides IFN-γ also proinflammatory cytokines such as tumor necrosis factor-alpha and -beta and thus mainly protect against intracellular pathogens. Th2 cells produce Interleukins (IL) IL-4, IL-5, IL-9, IL-10, and IL-13 and are important for the combat against extracellular pathogens. The newly identified Th17 cells produce IL-7A and F, and play an important role in autoimmunity.

Besides Th-1, Th-2 and Th-17 cells, the population of CD4+ T cells includes approximately 10% regulatory T cells playing an essential role in the dampening of immune responses, in the prevention of autoimmune diseases and in oral tolerance. Regulatory T cells can be subdivided in CD4-, CD25- and CTLA4-positive natural regulatory T cells (Treg) as well as Th3 and Tr1 cells, which are characterized by the production of TGF-β (Th3 cells) or IL-10 (Tr1 cells).

In addition to the coordination of cell-mediated immune response by Th-cells, the elimination of infected cells is essential. This task is fulfilled by cytotoxic T cells (CTL). They can use their T-cell receptor to bind both to endogenous and exogenous peptides presented on MHC class I molecules. As a co-receptor serves the glycoprotein CD8, wherein a second costimulatory signal via CD28 has to take place which interacts with CD80 or CD86 of APC. Even then it comes to a complete activation of CTL. The production of co-receptors CD80 and CD86, in the APC is excited via PAMPs derived from the pathogen included. Is now a foreign cell epitope presented by an APC and recognized by a CTL, so there may be a complete activation of CTL. This activated CTL may now lyse identically infected cells by releasing perforin and granzyme B. In addition to the direct control of the infected cell it also occurs a secretion of IFN-γ (Morandi et al., 2008) which enhances the activity of CTL and activates NK cells.

Another important representative of T cells in addition to the Th cells and CTL are the natural killer T cells (NKT cells). They do not bind with their T-cell receptor to peptides presented by MHC molecules but to lipids bound by the MHC-like Molecule CD1d. If an exogenous and endogenous lipid is presented and is recognized by NKT cells as pathogen a Th1 and/or Th2 cell-mediated defense reaction occurs dependent on the structure or conformation of the presented lipids.

The cell-mediated immune response plays an important role in the recognition and control of foreign tissue and, thus, contributes also to the rejection of transplanted tissue. In order to avoid graft rejection, a lifelong individual immunosuppressive treatment of transplant recipient is necessary, wherein it is distinguished between two treatment levels.

The initial induction therapy takes place directly after transplantation for a short period and rarely longer than three months. In this case, antibodies are administered which block cell surface receptors or lead to depletion of certain cell populations to prevent early rejection of the graft. For example, the anti-CD25 antibody basiliximab blocks the interleukin-2 receptors on activated T cells. The humanized monoclonal antibody alemtuzumab, however, binds to CD52 and leads to the depletion of T and B lymphocytes, NK cells and monocytes (Abboudi & Mcphee, 2012).

Maintenance therapy is a life-long therapy with a combination of different immunosuppressants which lead to a reduced inflammation and activation of lymphocytes to prevent the destruction of the graft by the immune system. Here, for example, corticosteroids with calcineurin inhibitors such as cyclosporine or tacrolimus and antiproliferative agents such as azathioprine or mycophenolate are used. Thus, the synthesis of interleukins and interferons as well as purine is blocked and co-stimulation by CD28 is prevented (Abboudi & Macphee, 2012).

In the administration of immunosuppressants it is not only important to weaken the immune response so that damage or rejection of the transplanted organ is prevented, but also that the functionality of the immune system is maintained to combat pathogens sufficiently. Thus, in case of an oversuppression of the immune system infections and reactivations of pathogens may occur which may lead to serious complications caused by the transplantation.

One example is infection with EBV, which, in the transplant recipient may be accompanied with a lymphoma-like disease in particular the post-transplant lymphoproliferative disease (PTLD) and often results in death of the patient (Hansen & Nielsen, 2012). Furthermore, a CMV reactivation occur which when untreated may cause serious and sometimes fatal complications such as CMV disease, graft loss, and opportunistic infections (Ljungman et al., 1986). In addition, immunosuppressants often have severe side effects which may lead, inter alia, to metabolic disorders, impaired hepatic and renal function, as well as gastrointestinal complaints. Thus, it is important to dose as accurately as possible immunosuppressants individually to prevent a rejection of the transplanted organ or injury through its own immune defense and simultaneously maintain the functional immune status for defense of infection.

However, the determination of the immune status does not only play a role in case of transplantations but also if the subject suffers from an auto-immune disease, such as rheumatism. Physicians usually treat autoimmune diseases with an immunosuppressive drug that decreases the activity of the immune system so it does not attack the person's own tissues or transplanted organs or tissues (e.g. islet transplants as treatment of T1D). The disadvantage of immunosuppressive drugs is that they not only suppress the attack on the patient's own cells (or transplanted cells) but also hinder the ability of the immune system to fight infectious diseases.

Moreover, it is important to analyze the immune status of subjects which are immunosuppressed due to an HIV (human immunodeficiency virus) infection, human subject who may suffer from systemic inflammatory response syndrome (SIRS), compensatory anti-inflammatory response syndrome (CARS)—both after sepsis or during pregnancy.

CARS is an immunologic phenomenon that increasingly was noticed to occur in sepsis. Like its precursor, SIRS CARS is a complex and incompletely defined pattern of immunologic responses to severe infection. While SIRS is a proinflammatory syndrome that seemed tasked with killing infectious organisms through activation of the immune system, CARS is a systemic deactivation of the immune system tasked with restoring homeostasis from an inflammatory state. Additionally, it has a distinct set of cytokines and cellular responses and may have a powerful influence on clinical outcomes in sepsis.

Since a determination of drug concentration in blood is not suitable enough for the assessment of the degree of immunosuppression (Levitsky, 2011), other methods of monitoring the immune system are required. One way is the monitoring of the cellular immune response which is possible limited by a number of available diagnostic products. It may be distinguished between a direct detection with quantification and analysis of the functionality of specific immune cells.

The ImmunKnow assay from Cylex is designed to measure the activity of CD4+ T cells as a marker of global immune-competence. This assay detects intracellular ATP synthesis in antigen-unspecific stimulated CD4+ cells isolated from whole blood utilizing magnetic beads.

One possibility for direct identification and quantification of peptide-specific T cells represent peptide-loaded HLA multimers as for example, offered by Beckman Coulter Inc. in the form of tetramers or by ProImmune Ltd. in the form of pentamers. These multimers are complexes from a fluorochrome conjugated with MHC molecules, which bind a specific peptide.

The multimers may thus only bind epitope-recognizing T helper or cytotoxic T cells depending on the class of the MHC molecule. Here, also, the disadvantage of this detection strategy becomes apparent, since the multimers are both epitope and HLA-specific and thus only a part of the T-cells may be detected. In addition, no statement regarding the functionality of the detected cells is possible. A further disadvantage is that in a more comprehensive verification of the T cells a wide range of multimers must be used, which is reflected in high costs. In addition, multimers are only available for a limited number of HLA alleles, majorly HLA class-I alleles.

The verification of the functionality of cells, however, is based on an ex vivo stimulation of heparinized blood or PBMC (mononuclear cells peripheral blood) with pathogen or disease-specific antigens. The detection of specifically stimulated cells takes place by determining production characteristic marker cytokines such as IFN-γ (Lucas & Gaudieri, 2012). The measurement of marker cytokines may be performed using various techniques by ELISA (Enzyme-linked immunosorbent assay), the ELISpot assay (enzyme-linked immunospot assay), multiplex bead assays or by flow cytometry using an intracellular cytokine staining or the secretion assay. Alternative methods for the detection of marker production are PCR, RT-qPCR or array-based technologies.

A commercially available test to verify the functionality of immune cells to CMV is the T-Track® CMV of Lophius Biosciences GmbH. This test is based on the stimulation of PBMC with urea formulated CMV proteins and the subsequent detection of IFN-γ secreting cells by ELISpot assay and allows the assessment of the influence of immunosuppressive treatment on the functionality of a wide spectrum of CMV protein-reactive T-cells (Th cells and CTL) and APC. A further test to monitor the immune response against CMV is QuantiFERON-CMV from Cellestis Inc. This, however, only verifies the functionality of CMV-specific T cells by stimulation of whole blood with a mixture of CMV peptides.

The detection of secreted IFN-γ is based on the ELISA technology. The disadvantage of both test systems is that they only may be used in CMV-seropositive patients to verify the functionality of the cellular immune response against CMV. The Quanitiferon CMV test also considered only the functionality of CMV-specific CTL and does not allow assessment of the overall status of CMV-reactive cells.

Thus, there is a need to develop a composition allowing the assessment of the functionality of the broadest possible spectrum of clinically relevant effector cell mediated immunity regardless of infection status and the constellation of HLA alleles.

This object is solved by the subject matter defined in the claims.

The following figures illustrate the present invention.

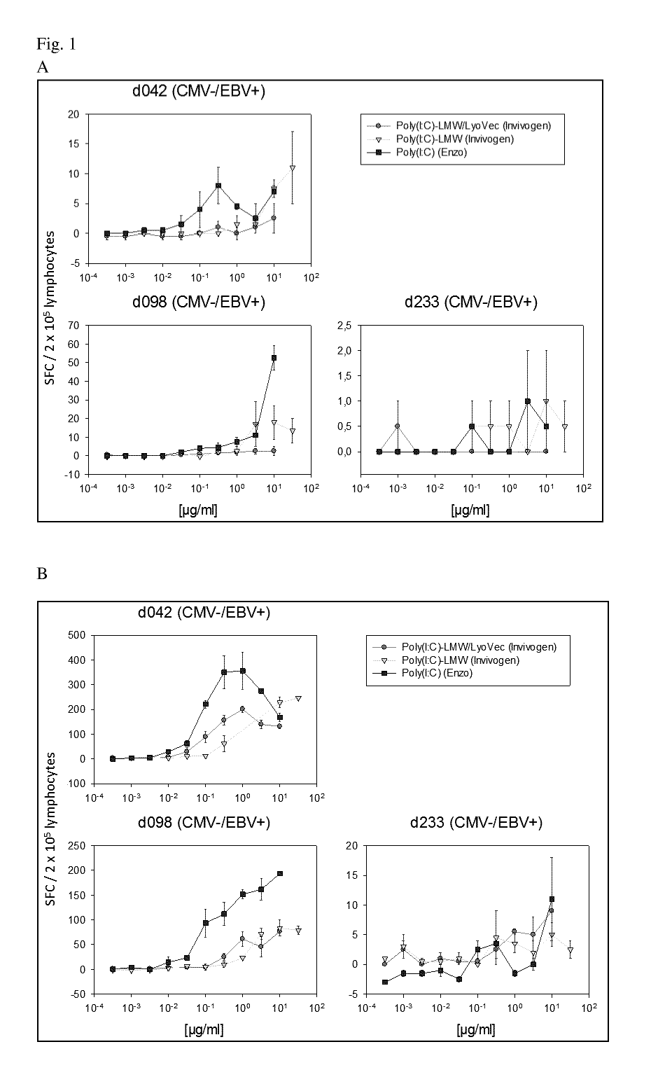

FIG. 1 shows the graphical course of the number of IFN-γ producing cells in SFC (spot-forming cells) of three donors d042, d098 and d233 in dependency of increasing concentrations of the three poly(I:C) variants, poly(I:C) (Enzo), poly(I:C)-LMW (Invivogen) and poly(I:C)-LMW/LyoVec (Invivogen). For each concentration value of poly(I:C) variant PBMC adjusted to 2×10⁵ lymphocytes were used and incubated for 19 hours at 37° C. and 5% CO₂. The number of IFN-γ secreting cells was determined by using the ELISpot assay in a one-step (FIG. 1 A) and two-step (FIG. 1 B) manner. Duplicate analyses were performed for each sample. The results showed are averages formed from the single values minus the averages formed from the single values of the unstimulated controls±standard deviation. The term in parentheses each refers to the companies InvivoGen San Diego, Calif., USA and Enzo Life Sciences GmbH, Lörrach from which the substances were purchased. Abbreviation: poly(I:C) means polyinosinic:polycytidylic acid.

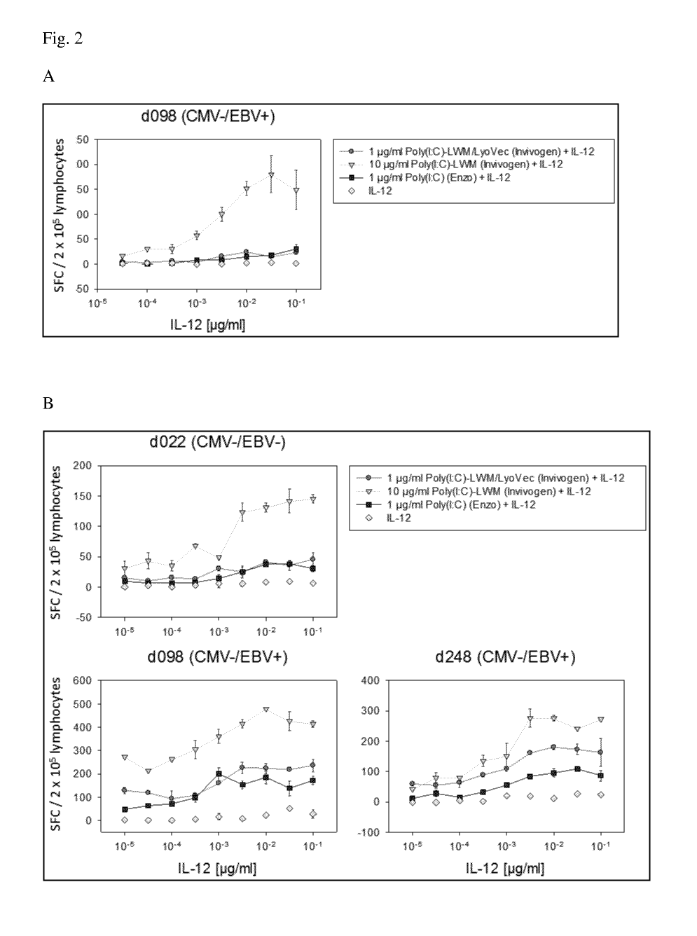

FIG. 2 shows the graphical course of the number of IFN-γ producing cells in SFC (spot-forming cells) of one donor d098 (A) and three donors d042, d098 and d233 (B), respectively in dependency of increasing concentrations of IL-12 alone or in combination with the three poly(I:C) variants, poly(I:C) (Enzo), poly(I:C)-LMW (Invivogen) and poly(I:C)-LMW/LyoVec (Invivogen). For each concentration value of IL-12 and combinations with poly(I:C) variant PBMC adjusted to 2×10⁵ lymphocytes were used and incubated for 19 hours at 37° C. and 5% CO₂. The number of IFN-γ secreting cells was determined by using the ELISpot assay in a one-step (FIG. 2 A) and two-step (FIG. 2 B) manner. Duplicate analyses were performed for each sample. The results showed are averages formed from the single values minus the averages formed from the single values of the unstimulated controls±standard deviation. The term in parentheses each refers to the companies InvivoGen San Diego, Calif., USA and Enzo Life Sciences GmbH, Lörrach from which the substances were purchased. Abbreviation: poly(I:C) means polyinosinic:polycytidylic acid.

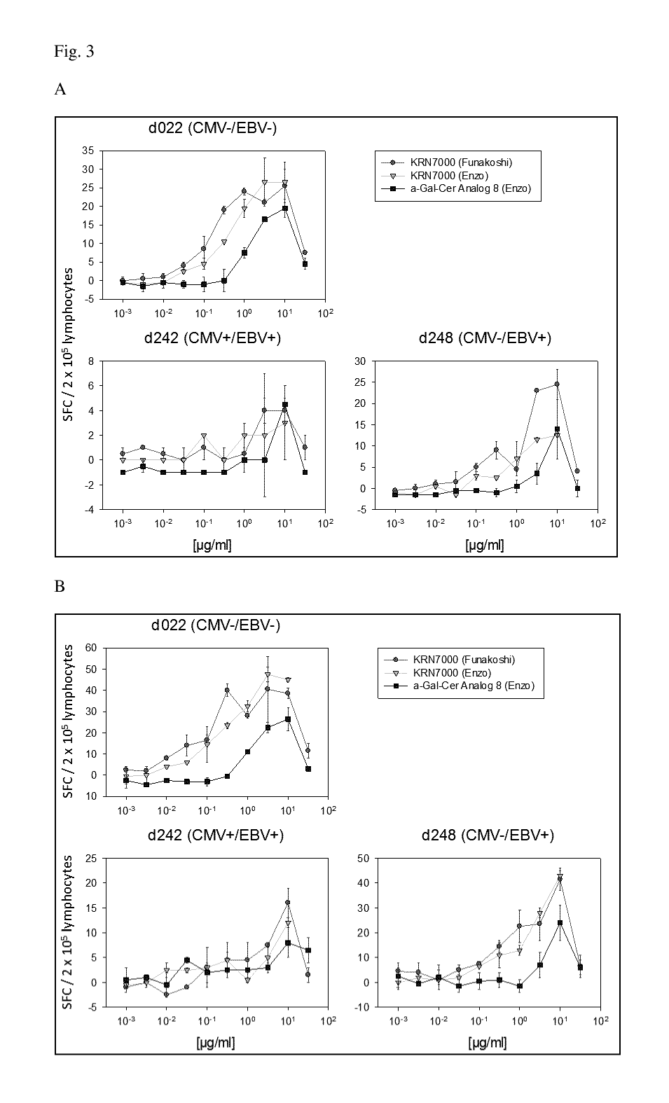

FIG. 3 shows the graphical course of the number of IFN-γ producing cells in SFC (spot-forming cells) of three donors d022, d242 and d248 in dependency of increasing concentrations of the three alpha Galactosylceramide variants, KRN7000 (Funakoshi), KRN7000 (Enzo) and a-Gal-Cer analogue 8 (Enzo). For each concentration value of three alpha Galactosylceramide variants PBMC adjusted to 2×10⁵ lymphocytes were used and incubated for 19 hours at 37° C. and 5% CO₂. The number of IFN-γ secreting cells was determined by using the ELISpot assay in a one-step (FIG. 3 A) and two-step (FIG. 3 B) manner. Duplicate analyses were performed for each sample. The results showed are averages formed from the single values minus the averages formed from the single values of the unstimulated controls±standard deviation. The term in parentheses each refers to the companies Funakoshi Co., Ltd., Tokyo, Japan and Enzo Life Sciences GmbH, Lörrach from which the substances were purchased. Abbreviation: a-Gal-Cer analogue 8 refers to alpha-Galactosylceramide analogue 8.

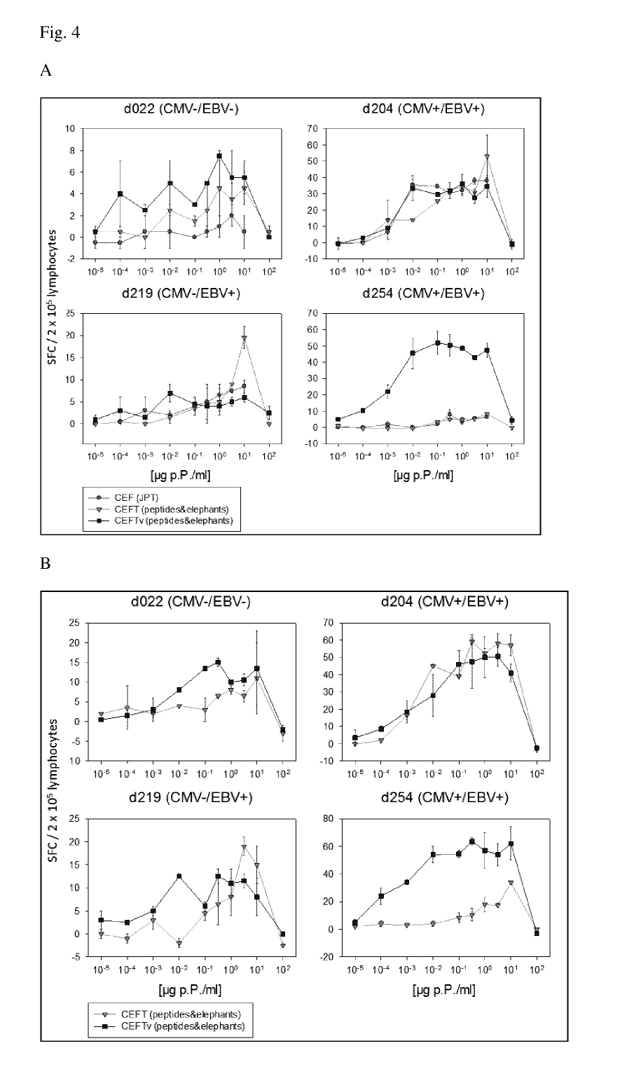

FIG. 4 shows the graphical course of the number of IFN-γ producing cells in SFC (spot-forming cells) of four donors d022, d204, d219 and d254 in dependency of increasing concentrations of the peptide pool CEF (JPT), CEFT (peptides&elephants) and CEFTv (peptides&elephants) (A) or CEFT (peptides&elephants) and CEFTv (peptides&elephants). For each concentration value of the peptide pool PBMC adjusted to 2×10⁵ lymphocytes were used and incubated for 19 hours at 37° C. and 5% CO₂. The number of IFN-γ secreting cells was determined by using the ELISpot assay in a one-step (FIG. 4 A) and two-step (FIG. 4 B) manner. Duplicate analyses were performed for each sample. The results showed are averages formed from the single values minus the averages formed from the single values of the unstimulated controls±standard deviation. The term in parentheses each refers to the companies JPT Innovative Peptide Solutions, Berlin and peptides&elephants GmbH, Potsdam, Germany from which the substances were purchased.

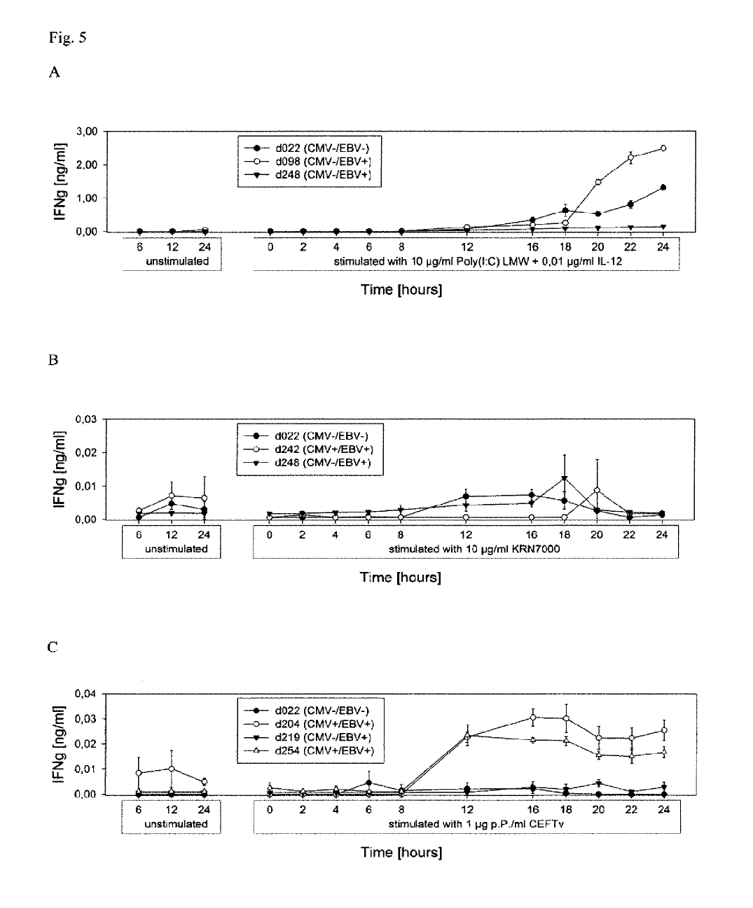

FIG. 5 shows the graphical course of the number of IFN-γ producing cells in SFC (spot-forming cells) of three donors d022, d098 and d248 in dependency of time after stimulation with 0.01 μg/ml IL-12 in combination with 10 μg/ml poly(I:C)-LMW (A) or of three donors d022, d242 and d248 in dependency of time after stimulation with 10 μg/ml KRN7000 (B) or of the four donors d022, d204, d219 and d254 after stimulation with 0.1 μg p.p./ml CEFTv (C) in comparison to unstimulated cells. For each stimulation preparation PBMC adjusted to 2×10⁵ lymphocytes were used and incubated for 19 hours at 37° C. and 5% CO₂ with poly(I:C)-LMW and KRN7000, respectively. The number of IFN-γ secreting cells was determined from the supernatant after centrifugation by using the ELISA. Triplicate analyses were performed for each sample. The results showed are averages±standard deviation. Abbreviation: poly(I:C) means polyinosinic:polycytidylic acid.

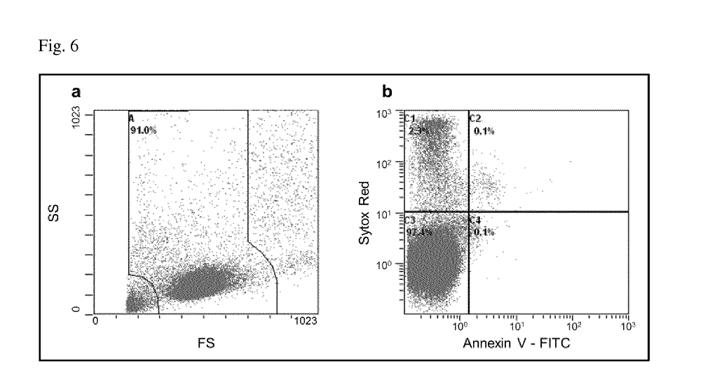

FIG. 6 shows the graphical representation of the gating procedure for determining and discrimination of vital and non-vital lymphocytes. Since the dead lymphocytes are distinct from vital lymphocytes due to their characteristic scattering light properties and diffract the light stronger, first the gate (a) was drawn in a broad manner to separate lymphocytes in general from debris. These cells registered by this gate A could be divided (b) in vital (C3), necrotic (C1), early-stage apoptotic (C4) or late-stage apoptotic (C2) cells by Sytox Red staining of the DNA and/or Annexin V-FITC staining of phophatidylserine.

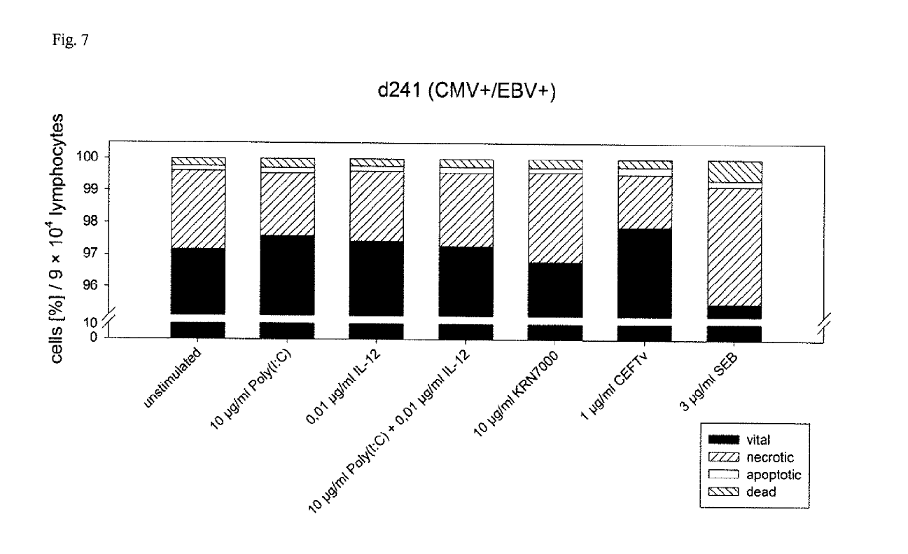

FIG. 7 shows in a column representation the results of a flow cytometric evaluation of the vitality of PBMC after 19 hours incubation with different stimulants. For each preparation PBMC adjusted to 1×10⁶ lymphocytes of donor d241 was given in a 5 ml round bottom tube and incubated for 19 hours at 37° C. and 5% CO₂ with one or two stimulants. After stimulation the Sytox Red staining and Annexin V-FITC staining of phosphatidylserine was performed and the cells were analyzed subsequently by flow cytometry. The composition of the measured lymphocytes of vital and non-vital cells is shown here in percent.

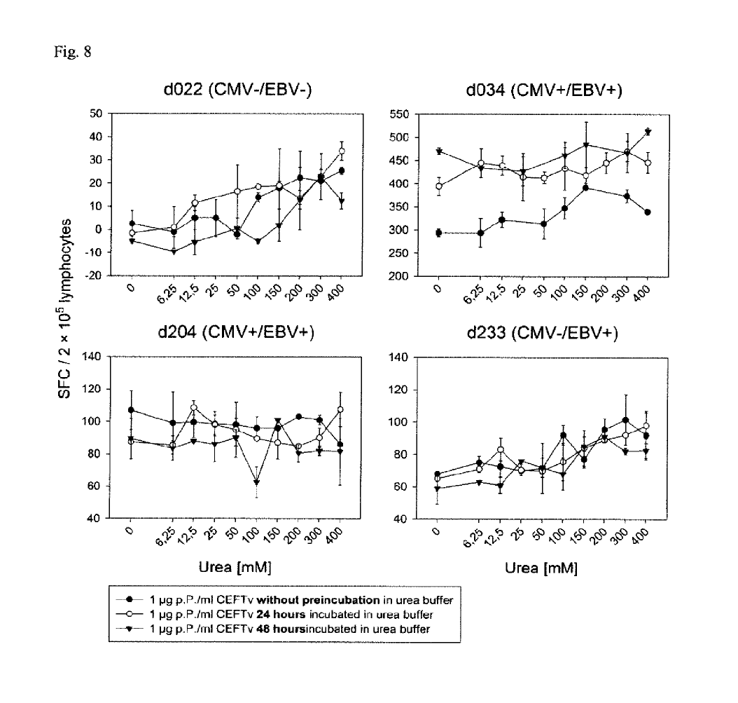

FIG. 8 shows the graphical course of the number of IFN-γ producing cells in SFC (spot-forming cells) of four donors d022, d034, d204 and d233 in dependency of increasing concentrations of urea used for pre-incubation of 1 μg p.p./ml CEFTv for 24 hours or 48 hours in comparison to CEFTv not preincubated with urea. For each stimulation preparation PBMC adjusted to 2×10⁵ lymphocytes were used and incubated for 19 hours at 37° C. and 5% CO₂. The number of IFN-γ secreting cells was determined by using the ELISpot assay in a two-step manner. Duplicate analyses were performed for each sample. The results showed are averages formed from the single values minus the averages formed from the single values of the unstimulated controls±standard deviation.

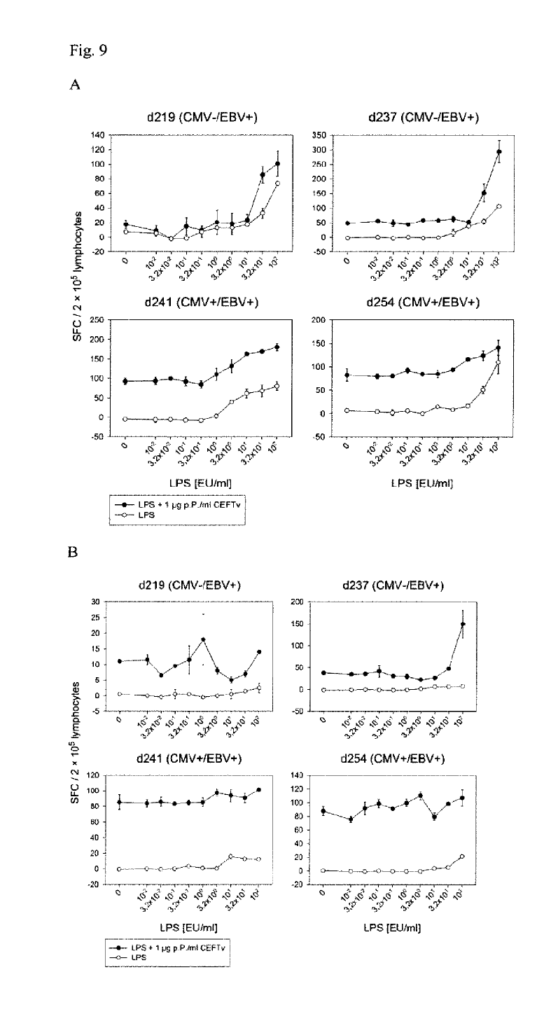

FIG. 9 shows the graphical course of the number of IFN-γ producing cells in SFC (spot-forming cells) of four donors d219, d237, d241 and d254 in dependency of increasing concentrations of LPS or LPS and 1 μg p.p./ml CEFTv used for pre-incubation of 1 μg p.p./ml CEFTv for 24 hours or 48 hours in comparison to CEFTv not preincubated with urea. For each stimulation PBMC adjusted to 2×10⁵ lymphocytes were used and incubated for 19 hours at 37° C. and 5% CO₂. The number of IFN-γ secreting cells was determined by using the ELISpot assay in a one-step (A) or two-step (B) manner. Duplicate analyses were performed for each sample. The results showed are averages formed from the single values minus the averages formed from the single values of the unstimulated controls±standard deviation.

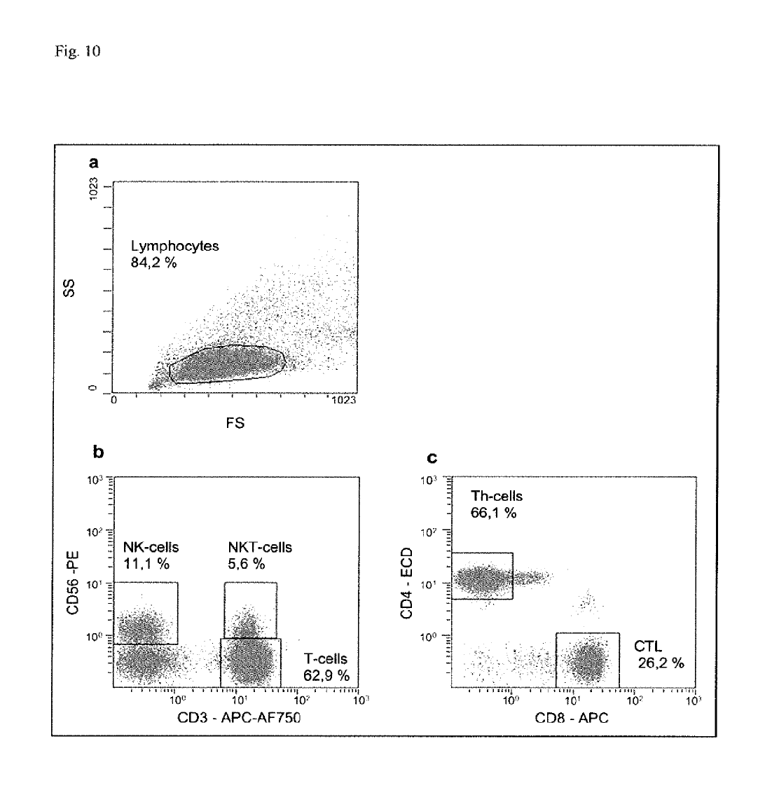

FIG. 10 shows a graphical representation of the gating procedure for distinction of lymphocyte populations. For evaluation the lymphocytes were selected by their characteristic scattering light properties from the overall population (a). Afterwards the NK, NKT-like and T cells were separated by the expression of CD56 and CD3 (b). Thereby it could be distinguished between CD3-CD56+NK cells, CD3+CD56+ NKT-like cells and CD3+CD56-T cells. At last the T cells were separated by their CD4 and CD8 expression in their subpopulations (c). Thereby the CD4+CD8− Th cells may be distinguished from CD4-CD8+ CTL.

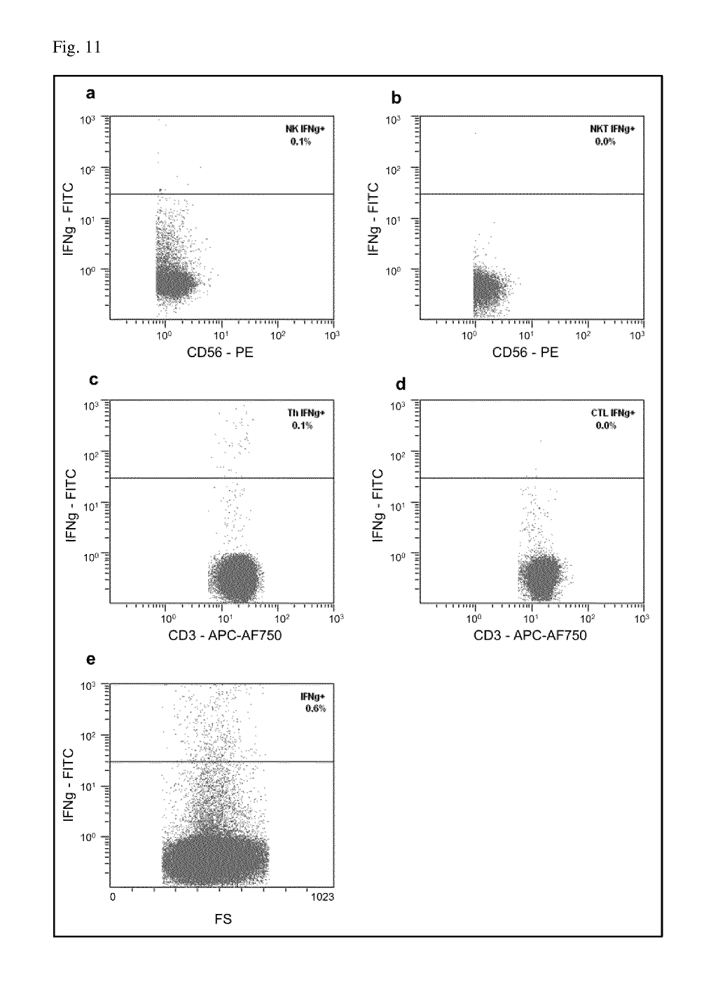

FIG. 11 shows a graphical representation of the gating procedure for determining of IFN-γ positive lymphocyte subpopulations. For individual evaluation a plot was formed for each of the isolated lymphocyte subpopulations (NK cells (a), NKT-like cells (b), Th cells (c), CTL (d)) and for total lymphocytes (e). The gating strategy of these populations is shown in FIG. 10. The gates for determination of IFN-γ positive cells were set utilizing isotype controls and non-stimulated cells. Cells stained bright enough to enter the IFN-γ gate were assessed to be positive.

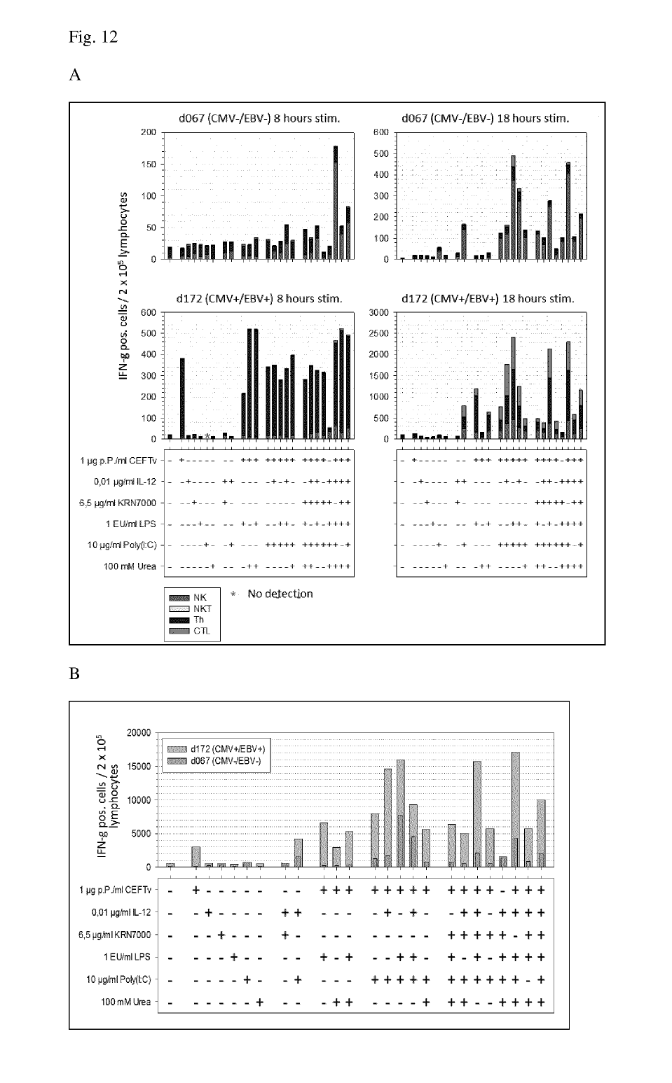

FIG. 12 shows a column diagram representing the number of IFN-γ producing lymphocyte subpopulations (A) or in lymphocytes (B) in SFC (spot-forming cells) of the two donors d067 and d172 after stimulation with different combinations of cell-specific stimulants for 8 hours or 18 hours. For each stimulation preparation PBMC adjusted to 1×10⁶ lymphocytes were given in a 5 ml round bottom tube and incubated at 37° C. and 5% CO₂ with the mentioned stimulants alone or in combinations for 8 and 18 hours. Six hours prior to the incubation end BFA was added to prevent the secretion of IFN-γ. After incubation period the CD antigens CD3, CD4, CD8 and CD56 were stained by fluorescence-labeled antibodies. After fixing and permeabilisation of the cells IFN-γ was intracellularly stained. The cells were stored at 4° C. in dark overnight and analyzed by flow cytometry on the next day. For evaluation the determined lymphocytes were normalized to 2×10⁵ lymphocytes and the composition of IFN-γ positive NK, NKT-like, Th cells and CTL are presented as stacked column diagrams (A). For the representation in FIG. 12 B the IFN-γ positive lymphocytes of the 8 hour and 18 hours stimulation were added and the results of the two donors opposed.

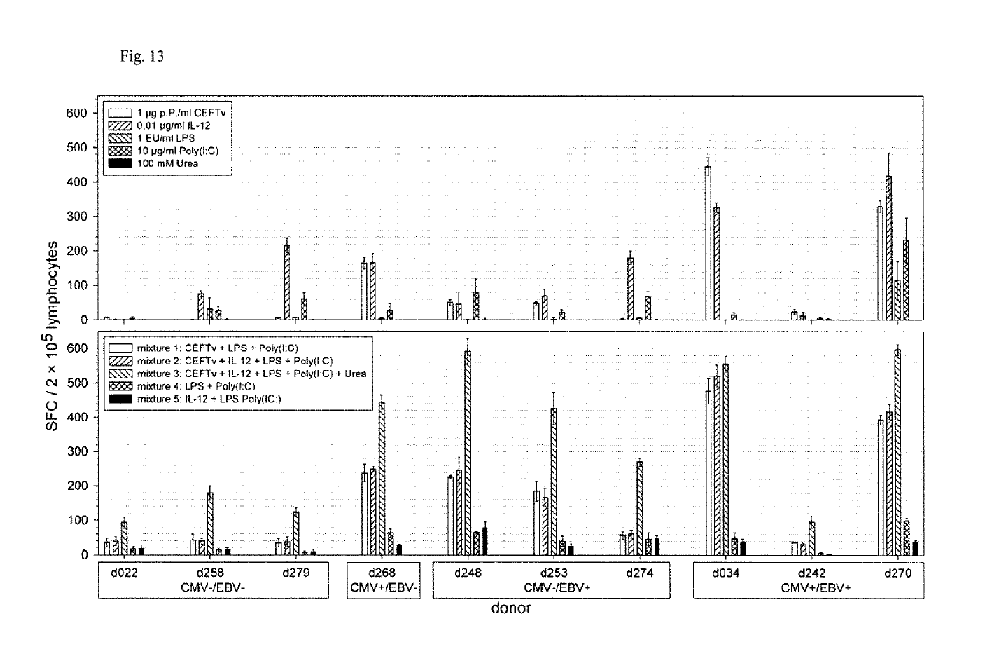

FIG. 13 shows a column diagram representing the number of IFN-γ producing cells in SFC (spot-forming cells) of the three CMV-negative/EBV-negative donors d022, d258 and d279, the CMV-positive/EBV-negative donor d268, the three CMV-negative/EBV-positive donors d248, d253 and d274 and the three CMV-positive/EBV-positive donors d034, d242 and d270 after stimulation with specific stimulants alone or in different combinations. For each stimulation preparation PBMC adjusted to 2×10⁵ lymphocytes were used and incubated for 19 hours at 37° C. and 5% CO₂. The number of IFN-γ secreting cells was determined by using the ELISpot assay in a one-step manner. Quadruple analyses were performed for each sample. The results showed are averages formed from the single values minus the averages formed from the single values of the unstimulated controls±standard deviation.

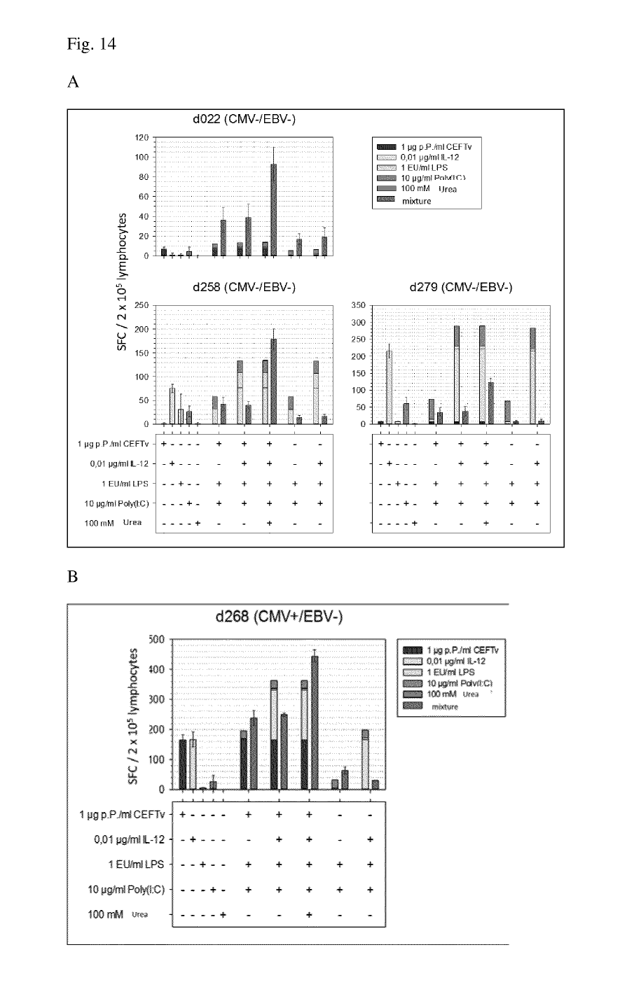

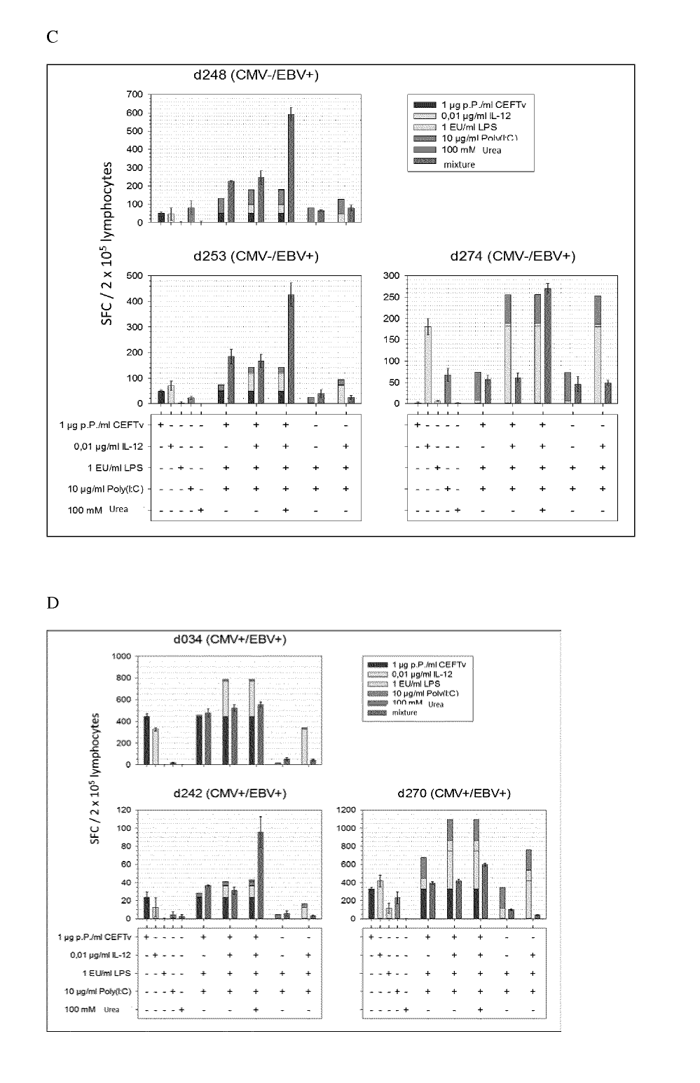

FIG. 14 shows a column diagram representing the number of IFN-γ producing cells in SFC (spot-forming cells) of the three CMV-negative/EBV-negative donors d022, d258 and d279 (A), the CMV-positive/EBV-negative donor d268 (B), the three CMV-negative/EBV-positive donors d248, d253 and d274 (C) and the three CMV-positive/EBV-positive donors d034, d242 and d270 (D) after stimulation with specific stimulants alone or in different combinations. For each stimulation preparation PBMC adjusted to 2×10⁵ lymphocytes were used and incubated for 19 hours at 37° C. and 5% CO₂. The number of IFN-γ secreting cells was determined by using the ELISpot assay in a one-step manner. The coloured column represents the addition of the numbers of IFN-γ secreting cells which were stimulated by the stimulants alone. The grey column represents the number of IFN-γ secreting cells after stimulation of PBMC with a combination of the stimulants. Quadruple analyses were performed for each sample. The results showed are averages formed from the single values minus the averages formed from the single values of the unstimulated controls±standard deviation. The stacked columns show the averages without standard deviation.

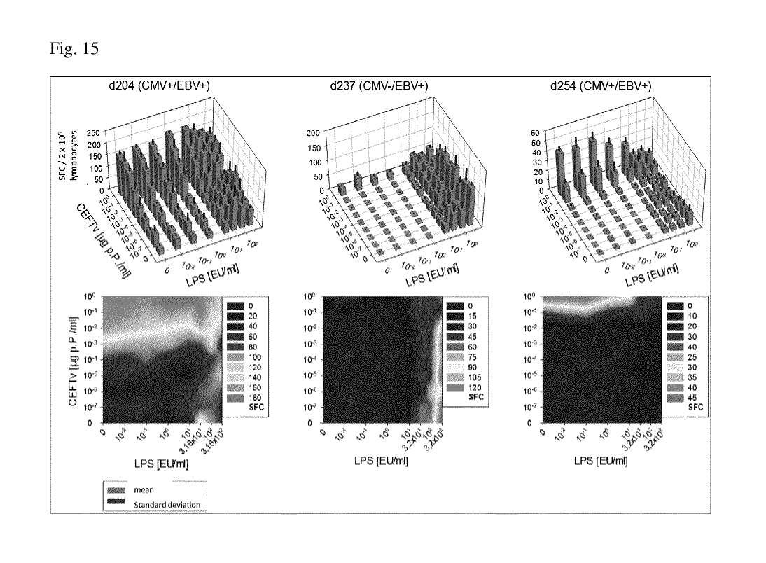

FIG. 15 shows graphical representations and column diagrams representing the number of IFN-γ producing cells in SFC (spot-forming cells) of the three donors d204, d237 and d254 after stimulation with LPS and CEFTv alone and in combination in different concentration ratios. CEFTv and LPS was each used alone, as well as titrated in half- and/or whole-logarithmic steps against each other. Per concentration, each PBMC adjusted to 2×10⁵ lymphocytes of three different donors d204, d237 and d254 were used and incubated for 19 hours at 37° C. and 5% CO₂. The number of IFN-γ secreting cells was determined by using the ELISpot assay in a one-step manner. Duplicate analyses were performed for each sample. The presented data are averages in the density-plot (below graphs) averages±standard deviation in the column diagram (upper graphs).

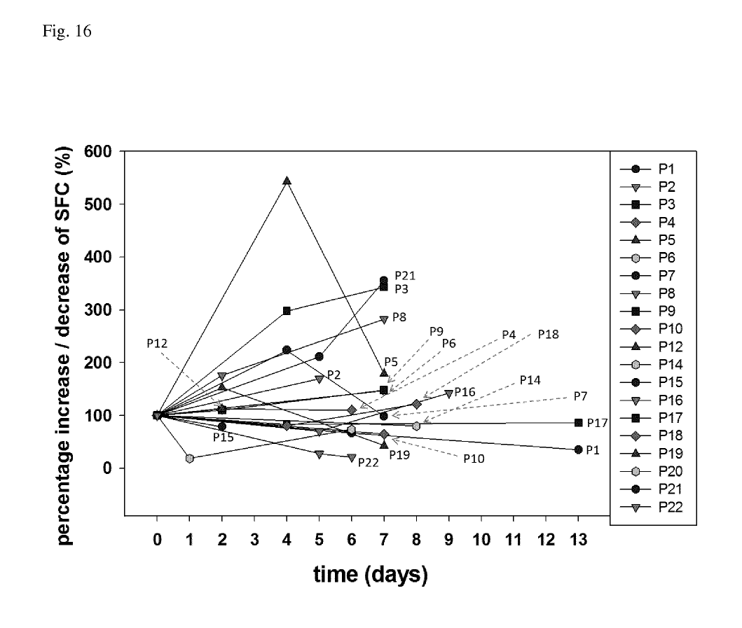

FIG. 16 shows the graphical course of the number of cocktail-responsive IFN-γ producing cells in SFC (spot-forming cells) of 20 rheumatism patients prior to and at indicated time points in the course of treatment with glucocorticoids and/or other immunomodulatory rheumatism drugs. For each stimulation with cocktail and SEB (positive control) (each at the indicated concentration) as well as the AIM-V medium (negative control) PBMC adjusted to 2×10⁵ lymphocytes were used and incubated for 19 hours at 37° C. and 5% CO₂. The number of IFN-γ secreting cells was determined by using the IFN-γ ELISpot assay as described in example 15. Quadruplicate analyses were performed for each sample. Mean values were formed from the quadruplicate measurements±standard deviation. Mean values of individual patients obtained at day 0 (prior to initiation of treatment) were set as 100% and means values of measurements obtained at subsequent measurements are shown as percentage increase/decrease compared to the mean value measured at day 0.

Abbreviation: P means patient.

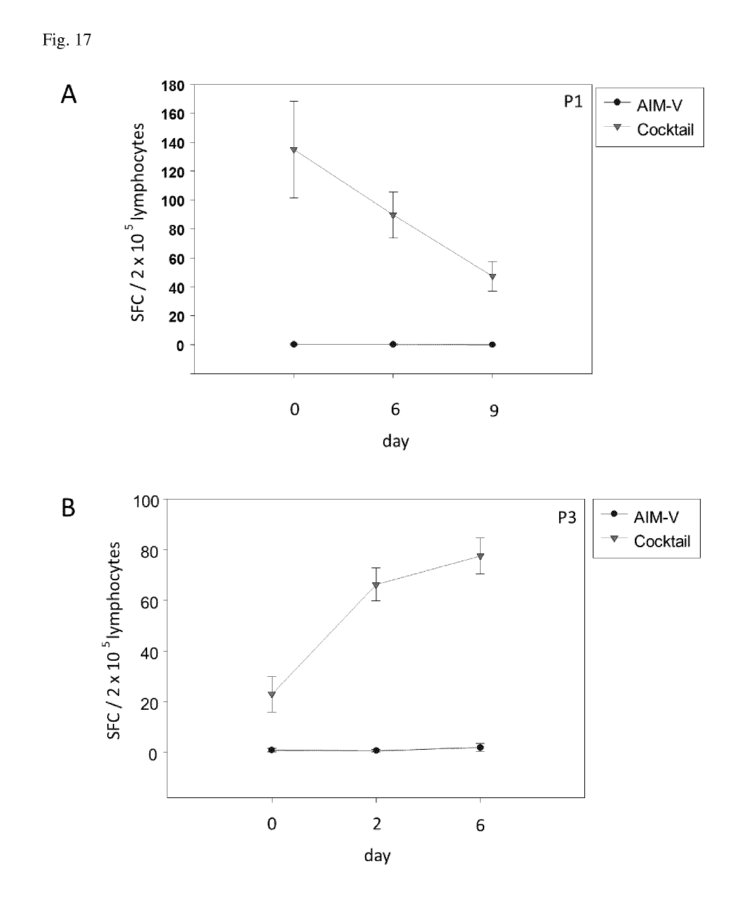

FIG. 17 shows the graphical course of the number of cocktail-responsive IFN-γ producing cells in SFC (spot-forming cells) of rheumatism patients p1 (A) and p3 (B) prior to and at indicated time points in the course of treatment with glucocorticoids and/or other immunomodulatory rheumatism drugs. For each stimulation with cocktail and SEB (positive control) (each at the indicated concentration) as well as the AIM-V medium (negative control) PBMC adjusted to 2×10⁵ lymphocytes were used and incubated for 19 hours at 37° C. and 5% CO₂. The number of IFN-γ secreting cells was determined by using the IFN-γ ELISpot assay as described in example 15. Quadruplicate analyses were performed for each sample. The results showed are mean values±standard deviation of the quadruplicate measurements. Abbreviation: AIM-V: Serum-free cell culture medium, P means patient, SFC means spot-forming cells.

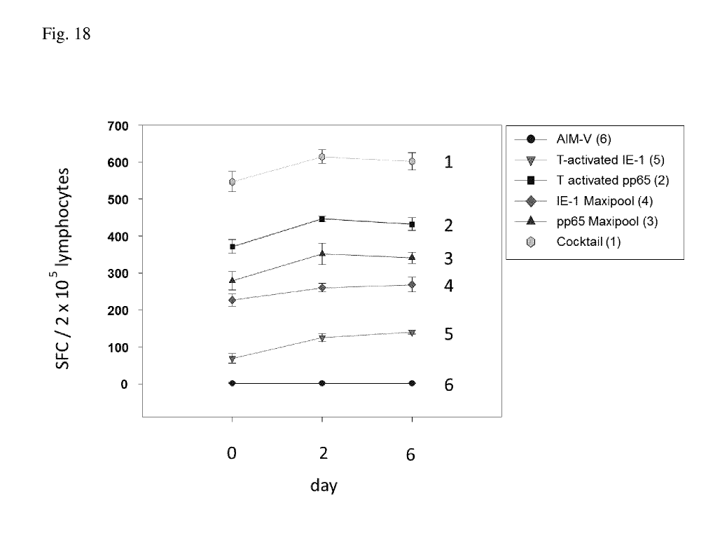

FIG. 18 shows the graphical course of the number of IFN-γ producing cells in SFC (spot-forming cells) of the CMV-seropositive rheumatism patient p4 prior to and at indicated time points in the course of treatment with glucocorticoids and/or other immunomodulatory rheumatism drugs. For stimulation with each (1) 50 μl cocktail working solution (2) 3 μg/ml T-activated pp65, (3) 1 μg/ml/peptide pp65 Maxipool (pool of 44 15-mer peptides with 11 aa overlap covering aa 366-546 of the CMV pp65 protein; strain AD169)), (4) 3 μg/ml/peptide IE-1 Maxipool (pool of 120 15-mer peptides with 11 aa overlap covering the complete CMV IE-1 protein; Towne strain)), and (5) 15 μg/ml T-activated IE-1 as well as the AIM-V medium (negative control) adjusted to 2×10⁵ lymphocytes were used and incubated for 19 hours at 37° C. and 5% CO₂. The number of IFN-γ secreting cells was determined by using the IFN-γ ELISpot assay as described in example 15. Quadruplicate analyses were performed for each sample. The results showed are mean values±standard deviation of the quadruplicate measurements. Abbreviations: aa: amino acid, AIM-V: CMV: cytomegalovirus; serum-free cell culture medium, P means patient, SFC means spot-forming cells.

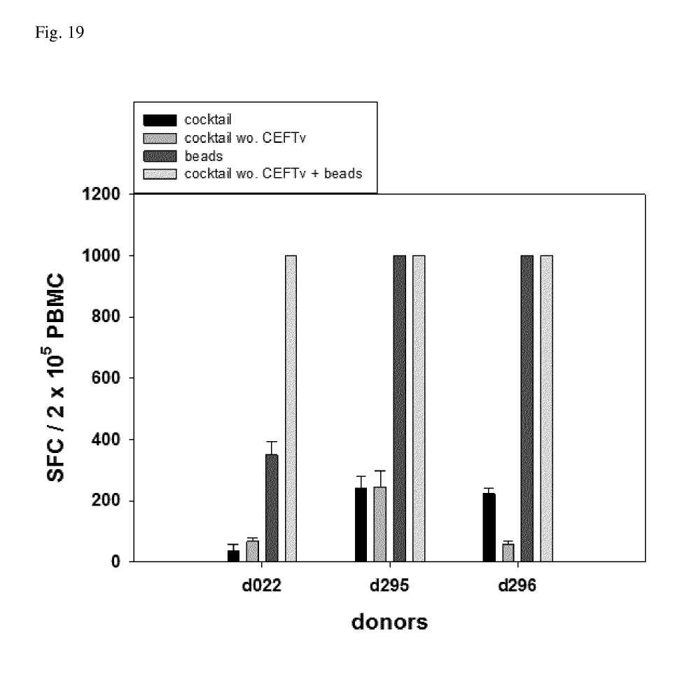

FIG. 19 shows the number of IFN-γ producing cells in SFC (spot-forming cells) in 2×10⁵ PBMC of three healthy individuals in response to a 19 hour stimulation at 37° C. and 5% CO2 with either the cocktail (original composition), the cocktail without the CEFTv peptides, the Dynabeads® Human T-Activator CD3/CD28 or the cocktail, where the CEFTv peptides were replaced by the Dynabeads® Human T-Activator CD3/CD28. The numbers of IFN-γ secreting cells were determined by using the IFN-γ ELISpot assay as described in example 15. Quadruplicate analyses were performed for each sample. The results showed are mean values±standard deviation of the quadruplicate measurements. Abbreviation: AIM-V: Serum-free cell culture medium, P means patient, SFC means spot-forming cells. SFC values of 1.000 are at the detection limit of the assay, thus indicated values of 1000 stand for values >1.000.

The term “T cells” as used herein refers to T lymphocytes, such as CD4+ T cells or CD8+ T cells or a mixture of CD4+ T cells, and CD8+ T cells, respectively. Herein the group of CD4+ T cells encompasses, T helper cells, such as T helper 1 (Th-1) cells, T helper 2 (Th-2) cells, T helper 17 (Th-17) cells, CD4+CD25+ regulatory T cells (Treg), Tr1 cells and T helper 3 (Th-3) cells. The group of CD8+ T cells comprises CD4-CD8+ cytotoxic T cells and T cells, which exhibit a CD4+CD8+ phenotype (CD4+CD8dim, CD4dimCD8bright or CD4hiCD8hi).

The term “capable to stimulate” cells as used herein refers to that the first or second substance according to the invention is capable to induce the transcription, expression, production and/or secretion of at least one immune effector molecule. The transcription, expression, production and/or secretion of at least one immune effector molecule may be detected by means of e.g. ELISpot, ELISA, FACS technology, multiplex bead assays, PCR, quantitative PCR (qPCR), reverse transcription quantitative real-time PCR (RT-qPCR) and/or microarray.

The term “cell-mediated immune response” as used herein refers to the direct and indirect effects of cells caused by the composition.

The term “p.p.” as used herein refers to the abbreviation per peptide.

The terms “control” or “control sample” as used herein refers to an experiment or test carried out to provide a standard, against which experimental results can be evaluated.

The term “immunosuppressed” as used herein refers to a condition of a subject who may suffer from a natural immunosuppression due to e.g. cancer, or from a bacterial, viral and/or fungal infection, systemic inflammatory response syndrome (SIRS), compensatory anti-inflammatory response syndrome (CARS)—both after sepsis or during pregnancy, pregnancy or increased age, or may suffer from immunosuppression due to therapeutic measures because the subject may have received a transplant and/or is receiving a transplant, suffers from rheumatoid arthritis or chronic inflammatory disease (e.g. Morbus Crohn, Colitis ulcerosa),

The term “species” as used herein refers to but not limited all kinds of pathogens, such as microorganisms like viruses, bacteria, fungi, nematodes, plasmodium.

The term “monitoring” as used here in refers to the use of the present invention to provide useful information about a subject or a subject's health, immune status, immune responsiveness, cell mediated immunity or disease status. “Monitoring” can include, determination of prognosis, risk-stratification, selection of drug therapy, assessment of ongoing drug therapy, prediction of outcomes, determining response to therapy, diagnosis of a disease or disease complication, following progression of a disease.

The term “detecting” as used herein refers to qualitatively or quantitatively determining the presence elevation or decrease in the level of at least one immune effector molecule by means of e.g. ELISpot, ELISA, FACS technology, multiplex bead assays, PCR, quantitative PCR (qPCR), reverse transcription quantitative real-time PCR (RT-qPCR) and/or microarray.

The term “enhancer” as used herein refers to a substance which increases the stimulatory activity of other substances, as e.g. the first and/or second substance on immune cells to release immune effector molecules.

The term “measuring” as used herein refers to the extent, quantity, quality, amount, or degree of at least one immune effector molecule, as determined by measurement or calculation.

The term “CD4+ T cell” as used herein refers to T helper cells, which either orchestrate the activation of macrophages and CD8+ T cells (Th-1 cells), the production of antibodies by B cells (Th-2 cells) or which have been thought to play an essential role in autoimmune diseases (Th-17 cells). In addition, the term “CD4+ T cells” also refers to regulatory T cells, which represent approximately 10% of the total population of CD4+ T cells. Regulatory T cells play an essential role in the dampening of immune responses, in the prevention of autoimmune diseases and in oral tolerance.

The terms “natural regulatory T cells” or “regulatory T cells” as used herein refer to Treg, Th3 and Tr1 cells. Treg are characterized by the expression of surface markers CD4, CD25, CTLA4 and the transcription factor Foxp3. Th3 and Tr1 cells are CD4+ T cells, which are characterized by the expression of TGF-β (Th3 cells) or IL-10 (Tr1 cells), respectively.

The terms “CD8+ T cell” or “CTL” as used herein refers to cytotoxic T cells recognizing and destructing degenerated, neoplastic and malignant cells as well as tissue and cells, which are infected by micro-organisms or parasites. CD8+ T cell or CD4-CD8+ T cells are also called CTL.

The term “antigen-presenting cell (APC)” as used herein refers to cells, which are capable of capturing, processing polypeptides and presenting fragments of these polypeptides (epitopes) to the immune system in association with MHC class I and MHC class II proteins. Particularly, the term “antigen-presenting cell (APC)” as used herein refers to professional APC such as dendritic cells, monocytes, macrophages and B cells, but also to non-professional APC such as neutrophiles, fibroblasts but also vascular epithelial cells and various epithelial, mesenchymal cells as well as microglia cells of the brain.

The term “immune cells” as used herein denotes lymphocytes with helper, cytolytic or regulatory properties such as, for example, CD4+ T cells, CD8+ T cells, CD4+CD8+ T cells, CD4+CD8dim T cells, CD4+ regulatory T cells, CD56+CD8+ and CD56-CD57+CD8+ NKT-like cells as well as CD16+CD56+NK cells. However, the term “immune cells” as used herein does not mean only immune cells held and multiplied in vitro in culture media but also immune cell populations taken from a healthy blood donor, patient or an animal as well as respectively purified immune cells.

The term “epitope” as used herein designates the region of a polypeptide which possesses antigen properties and for example serves as a recognition site of T cells or immunoglobulins. In the sense of this invention epitopes for example are those regions of polypeptides which are recognised by immune cells such as, for example, CD4+ T cells, CD8+ T cells, CD4+CD8+ T cells, CD4+CD8dim T cells, CD56+CD8+ and CD56-CD57+CD8+ NKT cells or CD4+ regulatory T cells. An epitope can comprise 3 or more amino acids. Usually, an epitope consists of at least 6 to 7 amino acids or, which is more common, 8 to 12 amino acids, or 13 to 18 amino acids. However, an epitope may also consist of more than 18 amino acids and—even more rarely—of more than 30 amino acids. The term “epitope” as used herein also comprises a unique spatial conformation for the epitope. This spatial conformation is obtained from the sequence of amino acids in the region of the epitope.

The term “polypeptide” or “protein” as used herein denotes a polymer of amino acids of arbitrary length. Preferably, the term “polypeptide” as used herein refers to a polymer of amino acids consisting of more than 6 amino acid residues. The term polypeptide also comprises the terms epitope, peptide, oligopeptide, protein, polyprotein and aggregates of polypeptides. Also included in this term are polypeptides which have post-translational modifications e.g. glycosylations, acetylations, phosphorylations and similar modifications as well as chemical modifications such as carbamoylations, thiocarbamoylations, substituted guanidine groups and similar modifications. This term furthermore comprises, for example, polypeptides which have one or a plurality of analogs of amino acids (e.g. unnatural amino acids), polypeptides with substituted links as well as other modifications which are state of the art, regardless of whether they occur naturally or are of non-natural origin.

The term “carbamoylation” as used herein means the transfer of the carbamoyl from a carbamoyl-containing molecule (e.g., carbamoyl phosphate) to an acceptor moiety such as an amino group, a carboxy group, a sulfhydryl group, a phosphate group, a hydroxyl group or a imidazole group. Moreover, the term “carbamoylation” as used herein further comprises the thiocarbamoylation of polypeptides.

The term “cyanate” as used herein refers to the anion NCO—derived from cyanic acid (HNCO) and any salt of cyanic acid. Moreover, the term “cyanate” as used herein refers to any organic compound containing the monovalent group —OCN and thus to any organic compound of the structure R—OCN, wherein R is any organic moiety. In particular the term “cyanate” as used herein refers also to isocyanate and isothiocyanate.

The present invention relates to a composition comprising
i) a first substance which is capable to stimulate T cells,
ii) a second substance which is capable to stimulate NK cells (natural killer cells), and
iii) lipopolysaccharide (LPS) and/or urea.

The first substance according to the present invention may stimulate T cells to produce at least one immune effector molecule. The capability of the first substance to stimulate T cells to produce at least one immune effector molecule may be determined by measuring the immune effector molecule(s), preferably cytokines, more preferably IFN-β, INF-γ, TNF-α, IL-2, IL-4, IL-5, IL-6, IL-10, IL-12, IL-13, GM-CSF, TGF-β, MIP1a, MIP1b, 4-1BB, preferably soluble and/or membrane bound, CD25, perforin and/or granzyme by means of an ELISA, an ELISPOT assay or by means of FACS technology, multiplex bead assays, PCR, quantitative PCR (qPCR), reverse transcription quantitative real-time PCR (RT-qPCR) and/or microarray. In a preferred embodiment the soluble immune effector molecules, preferably cytokines, more preferably IFN-β, INF-γ, TNF-α, IL-2, IL-4, IL-5, IL-6, IL-10, IL-12, IL-13, GM-CSF, TGF-β, MIP1a, MIP1b, 4-1BB, preferably soluble may be localized intracellular and/or extracellular.

In a preferred embodiment the composition according to the present invention comprises at least one, more preferably two or more first substances, most preferably 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90 or 100 first substances.

In one embodiment of the present invention, the first substance according to the present invention is derived from a pathogenic agent, associated with the disease condition or cancer or a toxicant. Alternatively, the infection, disease condition, cancer or toxicant may suppress cell-mediated immunity in which case any first substance to which the subject has been prior exposed could be employed.

In another embodiment, the first substance according to the present invention may be a stimulant. The stimulant according to the present invention comprises specific stimulants and/or unspecific stimulants. The specific stimulants are capable to stimulate the T cells specific via the T cell receptor, thus only a few T cells are stimulated. The unspecific stimulants are capable to stimulate the T cells independent of an epitope and thus, more T cells are stimulated. A specific stimulant according to the invention may be an antigen, preferably a protein, a polypeptide, a peptide and/or a peptide pool. An unspecific stimulant according to the invention may be an antibody, such as an anti-CD3 antibody, TGN1412, anti-CD28 antibody and/or anti-CD49 antibody and/or any combination thereof, and/or further stimulants such as, Staphylococcal enterotoxin B (SEB), plant lectins such as phytohemagglutinin (PHA) and pokeweed mitogen (PWM) and/or concanavalin A.

In a preferred embodiment the first substance according to the present invention is the pp65 protein and/or a fragment thereof. In a preferred embodiment the fragment of the pp65 protein comprises an immune dominant fragment. In another preferred embodiment the fragment of the pp65 protein may comprise at least the protein translocation domain. Such the protein translocation domain is rich of the amino acids lysine and arginine. In another preferred embodiment the fragment of the pp65 protein may comprise the amino acid sequence according to SEQ ID NO: 88.

In a preferred embodiment the pp65 protein has an amino acid sequence according to the SEQ ID NO: 83. In another preferred embodiment the pp65 protein fragment has an amino acid sequence according to SEQ ID NO: 84, 85 or 86. In another preferred embodiment the pp65 protein or fragment thereof may additionally comprise a tag, preferably a His-, S-, Strep-, Flag-, Avi-, Streptavidin-, MBP-, GST- and/or GFP-tag and/or at the C- and/or the N-terminal end of the protein. In another preferred embodiment the pp65 protein fragment has an amino acid sequence according to SEQ ID NO: 87.

Preferably, the first substance is an anti-CD3-antibody, more preferably an activating anti-CD3-antibody, most preferably OKT3. In another preferred embodiment the anti-CD3 antibody is used in combination with the anti-CD28 antibody or anti-CD49 antibody. In another preferred embodiment the first substance, preferably anti-CD3 antibody in combination with anti-CD28 or anti-CD49 antibody may be both and/or only one of them be immobilized on a carrier, such as a bead. In a preferred embodiment the first substance is a combination of anti-CD3 antibody and antiCD28 antibody provided on beads, more preferably provided as Dynabeads® Human T-Activator CD3/CD28 by the catalogue number 111.61D by the company ThermoFisher.

In another preferred embodiment the anti-CD3 antibody is used in combination with the anti-CD28 antibody or anti-CD49 antibody, wherein only the anti-CD3 antibody is provided on a bead.

In another preferred embodiment the anti-CD3 antibody is used in combination with the anti-CD28 antibody or anti-CD49 antibody provided on beads in a ratio of beads to cells of 5:1 to 1:1, more preferably 3:1 to 1:1, most preferably 1:1.

The first substance according to the present invention may be synthetic, recombinant or naturally occurring.

In a preferred embodiment the first substance according to the present invention is provided on a carrier or immobilized on beads. In another preferred embodiment the first substance according to the present invention is provided on beads in a ratio of beads to cells of 5:1 to 1:1, more preferably 3:1 to 1:1, most preferably 1:1.

It is also contemplated that the first substance is capable of eliciting a response from antigen-specific memory T cells in PBMC samples, irrespective of the infection status of the respective subject. The capability of the first substance to eliciting a response from antigen-specific memory T cells in PBMC samples may be determined by measuring the immune effector molecule(s).

The immune effector molecule(s) according to the invention may be a nucleic acid or a protein or polypeptide, in particular a RNA, a DNA, a nucleic acid fragment and is induced by contact and incubation with the composition according to the invention. The immune effector molecule according to the present invention may be a cytokine, such as a lymphokine, interleukin or chemokine, such as such as IFN-β, INF-γ, TNF-α, IL-2, IL-4, IL-5, IL-6, IL-10, IL-12, IL-13, GM-CSF, TGF-β, MIP1a, MIP1b, 4-1BB, preferably soluble and/or membrane bound, CD25, perforin and/or granzyme. Preferably the immune effector molecule is interferon gamma (IFN-γ). Alternatively or additionally, the immune effector molecule may be a co-stimulatory molecule, such as a member of the TNF-receptor or TNF-ligand superfamily, such as 4-1BB Ligand (4-1BBL), preferably soluble and/or membrane bound, OX40 ligand (OX40L), TNFSF (CD70), B7.1 (CD80), B7.2 (CD86), FcγRIII (CD16), FcγRII (CD32), FcγRI (CD64) or a further representative of the TNF/TNF-receptor and/or immunoglobulin superfamily or a member of the CXCL family e.g. CXCL9, CXCL10, CXCL11 or a member of the chemokine (C—C motif) ligand family e.g. CCL2, CCL 7, CCL8, CCL10 or IL1RN.

In a preferred embodiment the soluble immune effector molecules, preferably cytokines, more preferably IFN-β, INF-γ, TNF-α, IL-2, IL-4, IL-5, IL-6, IL-10, IL-12, IL-13, GM-CSF, TGF-β, MIP1a, MIP1b, 4-1BB, preferably soluble may be localized intracellular and/or extracellular.

The detection of the immune effector molecule(s) may be performed by means of PCR, qPCR (quantitative PCR), microarray, an ELISA, an ELISpot assay, multiplex bead assays or by means of FACS technology. Furthermore, according to the invention it is envisaged that the immune effector molecule is a further cytokine or chemokine produced by activated T cells or reactivated memory T cells.

In another embodiment the first substance is an antigen, preferably a protein, also referred to as protein antigen. In yet another embodiment the first substance according to the present invention is one or more peptides derived from a protein antigen. In a further embodiment the first substance according to the present invention is one or more peptides selected from a peptide pool. Preferably, the peptide pool comprises two or more peptides.

Another aspect of the present invention relates to a composition comprising a peptide pool according to the present invention.

A further aspect relates to a peptide pool according to the present invention.

In an embodiment the peptide pool according to the present invention may be used irrespective of the infection status of the subject. In a preferred embodiment, the peptide pool comprises antigens of CMV (Cytomegalovirus), EBV (Epstein Barr virus), VZV (Variella zoster virus). In a preferred embodiment the peptide pool according to the present invention may be used for determining the immune status of a subject irrespective of the infection status of the subject, preferably independent of the EBV-, VZV-, HCV, HCMV-, Influenza- and Clostridium tetani—serostatus of the subject.

The peptide may have any length, such as 6 or more, e.g. 6, 7, 8, 9, 10, 11, 12, 13, 14, 15, 16, 17, 18, 19, 20, 25, 26, 27, 28, 29, 30, 31, 32, 34, 35, 36, 37, 38, 39, 40, 50, 60, 70, 80, 90, 100, 110, 120, 130, 140, 150, 160, 170, 180, 190, 200 or more amino acids. The peptide may also be a fragment of a protein. Such a fragment may also have a length of 6, 7, 8, 9, 10, 11, 12, 13, 14, 15, 16, 17, 18, 19, 20, 25, 26, 27, 28, 29, 30, 31, 32, 34, 35, 36, 37, 38, 39, 40, 50, 60, 70, 80, 90, 100, 110, 120, 130, 140, 150, 160, 170, 180, 190, 200 or more amino acids.

In a preferred embodiment the peptide pool comprises 2 or more peptides, e.g. 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 50, 60, 70, 80, 90, 100, 110, 120, 130, 140, 105, 160, 170, 180, 190, 200 or more peptides, more preferably 20, 21, 22, 23, 24, 25, 26, 27, 28, 29 30, 31, 32, 34, 35, 35, 36, 37, 38, 39 or 40 peptides.

In a preferred embodiment the peptide pool comprises at least two peptides having a length ranging from 5 to 100 amino acids, more preferably having a length ranging from 5 to 50 amino acids, even more ranging from 9 to 50 amino acids, more preferably ranging from 20 to 40 or from 20 to 50 or 20 to 60, most preferably from 18 to 31 amino acids.

In a further preferred embodiment, the peptide pool comprises at least two peptides which are all or part of protein antigen derived from the influenza virus, Epstein-Barr virus, Cytomegalovirus and Clostridium tetani. In a more preferred embodiment, the peptide pool comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100 110, 120, 130, 140, 105, 160, 170, 180, 190, 200 or more peptides, more preferably 20, 21, 22, 23, 24, 25, 26, 27, 28, 29 30, 31, 32, 34, 35, 35, 36, 37, 38, 39 or 40 peptides which are all or part of a protein antigen derived from the influenza virus, Epstein-Barr virus, Cytomegalovirus and Clostridium tetani.

In a further preferred embodiment, the peptide pool comprises at least two peptides which are all or part of protein antigen derived from distinct species, preferably from two or more distinct species. In a further preferred embodiment, the peptide pool comprises at least two peptides which are all or part of protein antigen derived from two distinct species selected form the group consisting of the influenza virus, Epstein-Ban virus, Cytomegalovirus and Clostridium tetani.

In a further preferred embodiment the peptides may optionally be overlapping, such as the antigen derived peptides arrangements that are disclosed for example in EP 1 257 290 B2.

The peptide pool may be but is not limited to peptide mixtures as disclosed in WO2013000021.

In a further preferred embodiment the peptide pool comprises at least two peptides which are all or part of protein antigen selected from the group consisting of Influenza A, Influenza PB1, Influenza NP, Influenza M1, Influenza M, CMV pp65, EBV EBNA 1, EBV EBNA-3A, EBV EBNA-3B, EBV EBNA-3C, EBV BRLF-1, EBV BMLF1, EBV BZLF-1, EBV RTA, EBNA 4NP, EBV LMP1, EBV LMP2A, EBV LMP2B, and Tetanus Toxin Precursor.

Due to the use of such peptide pools providing mixtures of peptides derived from antigens of different pathogens, antigens are provided to which the most humans have been exposed to. Since most people have been exposed to these antigens it is expected that most of them possess a memory response to them.

In a preferred embodiment, the peptide pool is one or more of the peptide pools commercially available, more preferably an EF (Epstein Barr virus, Influenza virus), EFT- (Epstein Barr virus, Influenza virus, Clostridium tetani), CEF-(Cytomegalovirus, Epstein Barr virus, Influenza virus) or CEFT- (Cytomegalovirus, Epstein Barr virus, Influenza virus, Clostridium tetani) peptide pool as provided by JPT Innovative Peptide Solutions, Berlin; peptides&elephants GmbH, Potsdam; ProImmune Ltd., Oxford; Miltenyi Biotec GmbH, Bergisch Gladbach, Germany; Anaspec, EGT group, Fremont, Calif.; MABTECH AB, Germany; PANATecs GmbH, Heilbronn, Germany, CTL Europe GmbH, Germany and AXXORA DEUTSCHLAND GmbH.

In a preferred embodiment, the peptide pool is the CEF peptide pool according to the product code PM-CEF-S-120 as provided by JPT Innovative Peptide Solutions, Berlin, the CEFT peptide pool according to the order number CEF a or CEF c as provided by peptides&elephants GmbH, Potsdam; the CEFT peptide pool according to the product code PX-CEFT or the CEF peptide pool according to the product code PX-CEF as provided by ProImmune Ltd., Oxford; the CEF peptide pool according to the order number 130-098-426 as provided by Miltenyi Biotec GmbH, Bergisch Gladbach, Germany; the CEF peptide pool according to the catalog number 61036-05 as provided by Anaspec, EGT group, Fremont, Calif.; the CEF peptide pool according to the order number 3615-1 as provided by MABTECH AB, Germany; the CEFT peptide pool according to the catalog number PA-CEFT-001 or the EFT peptide pool according to the catalog number PA-EFT-001 as provided by PANATecs GmbH, Heilbronn, Germany and/or the CEF peptide pool according to the catalog number PT-PA-CEF-001-1, PT-PA-CEF-002-1, PT-PA-CEF-004-1 as provided by AXXORA DEUTSCHLAND GmbH.

In a particularly preferred embodiment the peptide pool is a CEFT pool comprising peptides that are extended at their respective N- and C-terminus by at least one, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 31, 32, 34, 35, 36, 37, 38, 39, 40, 50, 60, 70, 80, 90, 100 or more amino acids, preferably by five amino acids. Said extension preferably reflects the wild type amino acid sequence that is found at the N- and C-terminal end of the respective peptide when compared to the antigen from which this peptide was derived from.

In a preferred embodiment the peptide pool comprises at least two peptides according to SEQ ID NO: 1 to 82, more preferably according to SEQ ID NO: 1 to 27.

In a preferred embodiment the peptide pool comprises the peptides according to SEQ ID NO: 28 to 59 or the peptides according to SEQ ID NO: 28, 29, 30, 33, 34, 36, 37, 38, 41, 42, 43, 44, 46, 47, 48, 49, 50, 51, 52, 54, 56, 57 and 59, or the peptides according to SEQ ID NO: 28, 29, 30, 33, 34, 36, 37, 38, 41, 42, 43, 44, 46, 47, 48, 49, 50, 51, 52, 54, 56, 57, 59 and 61 to 63 or the peptides according to SEQ ID NO: 60 to 82 or the peptides according to SEQ ID NO:60, 64, 65, 68, 71, 72, 74, 81 and 82.

In a preferred embodiment the peptide pool comprises the peptides according to SEQ ID NO: 1 to 82, or the peptides according to SEQ ID NO: 28 to 59 or the peptides according to SEQ ID NO: 28 to 63 or the peptides according to SEQ ID NO: 28, 29, 30, 33, 34, 36, 37, 38, 41, 42, 43, 44, 46, 47, 48, 49, 50, 51, 52, 54, 56, 57 and 59, or the peptides according to SEQ ID NO: 28, 29, 30, 33, 34, 36, 37, 38, 41, 42, 43, 44, 46, 47, 48, 49, 50, 51, 52, 54, 56, 57, 59 and 61 to 63 or the peptides according to SEQ ID NO: 60 to 82 or the peptides according to SEQ ID NO: 60, 64, 65, 68, 71, 72, 74, 81 and 82, wherein the peptides are extended at their respective N- and C-terminus by at least one, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 31, 32, 34, 35, 36, 37, 38, 39, 40, 50, 60, 70, 80, 90, 100 or more amino acids, preferably by five amino acids. Said extension preferably reflects the wild type amino acid sequence that is found at the N- and C-terminal end of the respective peptide when compared to the antigen from which this peptide was derived from.

In a preferred embodiment the peptide pool is selected from one of the peptide pools EFT, CEF, CEFT and CEFTv as shown in Table 1, 2, 3, 4, 5 and 6. In the most preferred embodiment the peptide pool is CEFTv as shown in Table 6. In the preferred embodiment the peptide pool comprises the peptides according to SEQ ID NO: 1 to 27.

TABLE 1
Peptides of the CEF peptide pool:
CEF Peptide pool

| Peptide | | | HLA- |
|---|---|---|---|
| # | Amino acid sequence | Source | Restriction |
| 1 | VSDGGPNLY (SEQ ID NO: 28) | Influenza PB1 | HLA-A1 |
| 2 | CTELKLSDY (SEQ ID NO: 29) | Influenza NP | HLA-A1 |
| 3 | GILGFVFTL (SEQ ID NO: 30) | Influenza M1 | HLA-A2 |
| 4 | GLCTLVAML (SEQ ID NO: 33) | EBV BMLF1 | HLA-A2 |
| 5 | NLVPMVATV (SEQ ID NO: 34) | CMV pp65 | HLA-A2 |
| 6 | KTGGPIYKR (SEQ ID NO: 44) | Influenza NP | HLA-A68 |
| 7 | ILRGSVAHK (SEQ ID NO: 36) | Influenza NP | HLA-A3 |
| 8 | RVRAYTYSK (SEQ ID NO: 37) | EBV BRLF-1 | HLA-A3 |
| 9 | RLRAEAQVK (SEQ ID NO: 38) | EBV EBNA-3A | HLA-A3 |
| 10 | IVTDFSVIK (SEQ ID NO: 41) | EBV EBNA-3B | HLA-A11 |
| 11 | ATIGTAMYK (SEQ ID NO: 42) | EBV BRLF1 | HLA-A11 |
| 12 | DYCNVLNKEF (SEQ ID NO: 43) | EBV RTA | HLA-A24 |
| 13 | RPPIFIRRL (SEQ ID NO: 46) | EBV EBNA-3A | HLA-B7 |
| 14 | ELRSRYWAI (SEQ ID NO: 48) | Influenza NP | HLA-B8 |
| 15 | RAKFKQLL (SEQ ID NO: 49) | EBV BZLF-1 | HLA-B8 |
| 16 | FLRGRAYGL (SEQ ID NO: 50) | EBV EBNA 3A | HLA-B8 |
| 17 | QAKWRLQTL (SEQ ID NO: 51) | EBV EBNA 3 | HLA-B8 |
| 18 | SRYWAIRTR (SEQ ID NO: 54) | Influenza NP | HLA-B27 |
| 19 | RRIYDLIEL (SEQ ID NO: 56) | EBV EBNA 3C | HLA-B27 |
| 20 | YPLHEQHGM (SEQ ID NO: 57) | EBV EBNA 3A | HLA-B35 |
| 21 | EENLLDFVRF (SEQ ID NO: 59) | EBV EBNA-3C | HLA-B44 |
| 22 | EFFWDANDIY (SEQ ID NO: 52) | CMV pp65 | HLA-B44 |
| 23 | TPRVTGGGAM (SEQ ID NO: 47) | CMV pp65 | HLA-B7 |

TABLE 2
Peptides of the CEFT peptide pool:
CEFT Peptide pool

| Peptide | | | HLA- |
|---|---|---|---|
| # | Amino acid sequence | Source | Restriction |
| 1 | VSDGGPNLY (SEQ ID NO: 28) | Influenza PB 1 | HLA-A1 |
| 2 | CTELKLSDY (SEQ ID NO: 29) | Influenza NP | HLA-A1 |
| 3 | GILGFVFTL (SEQ ID NO: 30) | Influenza M1 | HLA-A2 |
| 4 | GLCTLVAML (SEQ ID NO: 33) | EBV BMLF1 | HLA-A2 |
| 5 | NLVPMVATV (SEQ ID NO: 34) | CMV pp65 | HLA-A2 |
| 6 | KTGGPIYKR (SEQ ID NO: 44) | Influenza NP | HLA-A68 |
| 7 | ILRGSVAHK (SEQ ID NO: 36) | Influenza NP | HLA-A3 |
| 8 | RVRAYTYSK (SEQ ID NO: 37) | EBV BRLF-1 | HLA-A3 |
| 9 | RLRAEAQVK (SEQ ID NO: 38) | EBV EBNA-3A | HLA-A3 |
| 10 | IVTDFSVIK (SEQ ID NO: 41) | EBV EBNA-3B | HLA-A11 |
| 11 | ATIGTAMYK (SEQ ID NO: 42) | EBV BRLF1 | HLA-A11 |
| 12 | DYCNVLNKEF (SEQ ID NO: 43) | EBV RTA | HLA-A24 |
| 13 | RPPIFIRRL (SEQ ID NO: 46) | EBV EBNA-3A | HLA-B7 |
| 14 | ELRSRYWAI (SEQ ID NO: 48) | Influenza NP | HLA-B8 |
| 15 | RAKFKQLL (SEQ ID NO: 49) | EBV BZLF-1 | HLA-B8 |
| 16 | FLRGRAYGL (SEQ ID NO: 50) | EBV EBNA 3A | HLA-B8 |
| 17 | QAKWRLQTL (SEQ ID NO: 51) | EBV EBNA 3 | HLA-B8 |
| 18 | SRYWAIRTR (SEQ ID NO: 54) | Influenza NP | HLA-B27 |
| 19 | RRIYDLIEL (SEQ ID NO: 56) | EBV EBNA 3C | HLA-B27 |
| 20 | YPLHEQHGM (SEQ ID NO: 57) | EBV EBNA 3A | HLA-B35 |
| 21 | EENLLDFVRF (SEQ ID NO: 59) | EBV EBNA-3C | HLA-B44 |
| 22 | EFFWDANDIY (SEQ ID NO: 52) | CMV pp65 | HLA-B44 |
| 23 | TPRVTGGGAM (SEQ ID NO: 47) | CMV pp65 | HLA-B7 |
| 24 | QYIKANSKFIGITEL (SEQ ID NO: | Tetanus Toxin | HLA-Klasse II |
| | 60) | Precursor | Allele not |
| | | | determined |
| 25 | FNNFTVSFWLRVPKVSASHLE (SEQ | Tetanus Toxin | HLA-DRB1 |
| | ID NO: 61) | Precursor | |
| 26 | KFIIKRYTPNNEIDSF | Tetanus Toxin | HLA-DPB1 |
| | (SEQ ID NO: 62) | Precursor | HLA-DRB1 |
| 27 | VSIDKFRIFCKALNPK | Tetanus Toxin | HLA-DRB1 |
| | (SEQ ID NO: 63) | Precursor | |

TABLE 3
Peptides of the PX-CEF peptide pool

| | Amino acid | | | |
|---|---|---|---|---|
| Peptide # | sequence | Source | Protein | HLA allele |
| 1 | VSDGGPNLY | Influenza A | RNA polymerase | HLA-A1 |
| | (SEQ ID NO: 28) | | | |
| 2 | CTELKLSDY | Influenza A | Nucleoprotein | HLA-A1 |
| | (SEQ ID NO: 29) | | | |
| 3 | GILGFVFTL | Influenza A | Matrix Protein | HLA-A2 |
| | (SEQ ID NO: 30) | | | |
| 4 | FMYSDFHFI | Influenza A | Polymerase PA | HLA-A2 |
| | (SEQ ID NO: 31) | | | |
| 5 | CLGGLLTMV | EBV | LMP2A | HLA-A2 |
| | (SEQ ID NO: 32) | | | |
| 6 | GLCTLVAML | EBV | BMLF1 | HLA-A2 |
| | (SEQ ID NO: 33) | | | |
| 7 | NLVPMVATV | HCMV | pp65 | HLA-A0201 |
| | (SEQ ID NO: 34) | | | |
| 8 | RVLSFIKGTK | Influenza A | Nucleoprotein | HLA-A3 |
| | (SEQ ID NO: 35) | | | |
| 9 | ILRGSVAHK | Influenza A | Nucleoprotein | HLA-A3 |
| | (SEQ ID NO: 36) | | | |
| 10 | RVRAYTYSK | EBV | BRLF1 | HLA-A3 |
| | (SEQ ID NO: 37) | | | |
| 11 | RLRAEAQVK | EBV | EBNA3A | HLA-A3 |
| | (SEQ ID NO: 38) | | | |
| 12 | SIIPSGPLK | Influenza A | Matrix Protein | HLA- |
| | (SEQ ID NO: 39) | | | A3/A11/A68 |
| 13 | AVFDRKSDAK | EBV | EBNA-3B | HLA-A11 |
| | (SEQ ID NO: 40) | | | |
| 14 | IVTDFSVIK | EBV | EBNA-3B | HLA-A11 |
| | (SEQ ID NO: 41) | | | |
| 15 | ATIGTAMYK | EBV | BRLF1 | HLA-A11 |
| | (SEQ ID NO: 42) | | | |
| 16 | DYCNVLNKEF | EBV | BRLF1 | HLA-A24 |
| | (SEQ ID NO: 43) | | | |
| 17 | KTGGPIYKR | Influenza A | Nucleoprotein | HLA-A68 |
| | (SEQ ID NO: 44) | | | |
| 18 | LPFDKTTVM | Influenza A | Nucleoprotein | HLA-B7 |
| | (SEQ ID NO: 45) | | | |
| 19 | RPPIFIRRL | EBV | EBNA 3A | HLA-B7 |
| | (SEQ ID NO: 46) | | | |
| 20 | TPRVTGGGAM | HCMV | pp65 | HLA-B7 |
| | (SEQ ID NO: 47) | | | |
| 21 | ELRSRYWAI | Influenza A | Nucleoprotein | HLA-B8 |
| | (SEQ ID NO: 48) | | | |
| 22 | RAKFKQLL | EBV | BZLF1 | HLA-B8 |
| | (SEQ ID NO: 49) | | | |
| 23 | FLRGRAYGL | EBV | EBNA 3A | HLA-B8 |
| | (SEQ ID NO: 50) | | | |
| 24 | QAKWRLQTL | EBV | EBNA 3A | HLA-B8 |
| | (SEQ ID NO: 51) | | | |
| 25 | EFFWDANDIY | HCMV | PP65 | HLA-B12/B44 |
| | (SEQ ID NO: 52) | | | |
| 26 | SDEEEAIVAYTL | HCMV | IE-1 | HLA-B18 |
| | (SEQ ID NO: 53) | | | |
| 27 | SRYWAIRTR | Influenza A | Nucleoprotein | HLA-B27 |
| | (SEQ ID NO: 54) | | | |
| 28 | ASCMGLIY | Influenza A | Matrix Protein | HLA-B27 |
| | (SEQ ID NO: 55) | | | |
| 29 | RRIYDLIEL | EBV | EBNA 3C | HLA-B27 |
| | (SEQ ID NO: 56) | | | |
| 30 | YPLHEQHGM | EBV | EBNA 3A | HLA-B35 |
| | (SEQ ID NO: 57) | | | |
| 31 | IPSINVHHY | HCMV | pp65 | HLA-B35 |
| | (SEQ ID NO:58) | | | |
| 32 | EENLLDFVRF | EBV | EBNA3C | HLA-B44 |
| | (SEQ ID NO: 59) | | | |

TABLE 4
Peptides of the CEFT II peptide pool

| Peptide | | |
|---|---|---|
| # | Amino acid sequence | Source |
| 1 | FVFTLTVPSER (SEQ ID NO: 64) | Influenza A |
| 2 | SGPLKAEIAQRLEDV (SEQ ID NO: 65) | Influenza A |
| 3 | YDVPDYASLRSLVASS (SEQ ID NO: 66) | Influenza A |
| 4 | PYYTGEHAKAIGN (SEQ ID NO: 67) | Influenza B |
| 5 | GQIGNDPNRDIL (SEQ ID NO: 68) | Tetanus |
| 6 | PKYVKQNTLKLA (SEQ ID NO: 69) | Influenza A |
| 7 | PKYVKQNTLKLAT (SEQ ID NO: 70) | Influenza A |
| 8 | DRLRRDQKS (SEQ ID NO: 71) | Influenza A |
| 9 | AGLTLSLLVICSYLFISRG | EBV |
| | (SEQ ID NO: 72) | |
| 10 | QYIKANSKFIGITEL (SEQ ID NO: 60) | Tetanus |
| 11 | QYIKANSKFIGITE (SEQ ID NO: 73) | Tetanus |
| 12 | FNNFTVSFWLRVPKVSASHLE (SEQ ID | Tetanus |
| | NO: 61) | |
| 13 | TSLYNLRRGRALA (SEQ ID NO: 74) | EBV |
| 14 | KFIIKRYTPNNEIDSF (SEQ ID NO: 62) | Tetanus |
| 15 | VSIDKFRIFCKALNPK (SEQ ID NO: 63) | Tetanus |
| 16 | VPGLYSPCRAFFNKEELL | EBV |
| | (SEQ ID NO: 75) | |
| 17 | DKREMWMACIKELH (SEQ ID NO: 76) | Cytomegalovirus |
| 18 | TGHGARTSTEPTTDY (SEQ ID NO: 77) | EBV |
| 19 | KELKRQYEKKLRQ (SEQ ID NO: 78) | EBV |
| 20 | RGYFKMRTGKSSIMRS (SEQ ID NO: 79) | Influenza A |
| 21 | TVFYNIPPMPL (SEQ ID NO: 80) | EBV |
| 22 | AEGLRALLARSHVER (SEQ ID NO: 81) | EBV |
| 23 | PGPLRESIVCYFMVFLQTHI (SEQ ID | EBV |
| | NO: 82) | |

TABLE 5
Peptides of the EFT peptide pool

| Peptide | Amino acid sequence | Source |
|---|---|---|
| # | | |
| 1 | FVFTLTVPSER (SEQ ID NO: 64) | Influenza A |
| 2 | SGPLKAEIAQRLEDV | Influenza A |
| | (SEQ ID NO: 65) | |
| 3 | GQIGNDPNRDIL (SEQ ID NO: 68) | Tetanus |
| 4 | DRLRRDQKS (SEQ ID NO: 71) | Influenza A |
| 5 | AGLTLSLLVICSYLFISRG | EBV |
| | (SEQ ID NO: 72) | |
| 6 | QYIKANSKFIGITEL (SEQ ID NO: 60) | Tetanus |
| 7 | TSLYNLRRGRALA (SEQ ID NO: 74) | EBV |
| 8 | AEGLRALLARSHVER (SEQ ID NO: 81) | EBV |
| 9 | PGPLRESIVCYFMVFLQTHI (SEQ ID | EBV |
| | NO: 82) | |

TABLE 6
Peptides of the CEFTv peptide pool:
CEFTv Peptide pool

| Peptide | | | HLA- |
|---|---|---|---|
| # | Amino acid sequence | Source | Restriction |
| 1v | KAGLLVSDGGPNLYNIRNL | Influenza PB1 | HLA-A1 |
| | (SEQ ID NO: 1) | | |
| 2v | FYIQMCTELKLSDYEGRLI | Influenza NP | HLA-A1 |
| | (SEQ ID NO: 2) | | |
| 3v | SPLTKGILGFVFTLTVPSE | Influenza Ml | HLA-A2 |
| | (SEQ ID NO: 3) | | |
| 4v | AIQNAGLCTLVAMLEETIF | EBV BMLF1 | HLA-A2 |
| | (SEQ ID NO: 4) | | |
| 5v | GILARNLVPMVATVQGQNL | CMV pp65 | HLA-A2 |
| | (SEQ ID NO: 5) | | |
| 6v | GKDPKKTGGPIYKRVDRKW | Influenza NP | HLA-A68 |
| | (SEQ ID NO: 6) | | |
| 7v | ARSALILRGSVAHKSCLPA | Influenza NP | HLA-A3 |
| | (SEQ ID NO: 7) | | |
| 8v | LLKHSRVRAYTYSKVLGVD | EBV BRLF-1 | HLA-A3 |
| | (SEQ ID NO: 8) | | |
| 9v | RDRLARLRAEAQVKQASVE | EBV EBNA-3A | HLA-A3 |
| | (SEQ ID NO: 9) | | |
| 10v | KKCRAIVTDFSVIKAIEEE | EBV EBNA-3B | HLA-A11 |
| | (SEQ ID NO: 10) | | |
| 11V | RVMIPATIGTAMYKLLKHS | EBV BRLF1 | HLA-A11 |
| | (SEQ ID NO: 11) | | |
| 12v | GSLVSDYCNVLNKEFTAGSV | EBV RTA | HLA-A24 |
| | (SEQ ID NO: 12) | | |
| 13v | GPKVKRPPIFIRRLHRLLL | EBV EBNA-3A | HLA-B7 |
| | (SEQ ID NO: 13) | | |
| 14v | DSNTLELRSRYWAIRTRSG | Influenza NP | HLA-B8 |
| | (SEQ ID NO: 14) | | |
| 15v | AARKSRAKFKQLLQHYRE | EBV BZLF-1 | HLA-B8 |
| | (SEQ ID NO: 15) | | |
| 16v | AWNAGFLRGRAYGLDLLRT | EBV EBNA 3A | HLA-B8 |
| | (SEQ ID NO: 16) | | |
| 17v | ASRRDQAKWRLQTLAAGWP | EBV EBNA 3 | HLA-B8 |
| | (SEQ ID NO: 17) | | |
| 18v | TLELRSRYWAIRTRSGGNT | Influenza NP | HLA-B27 |
| | (SEQ ID NO: 18) | | |
| 19v | WQRRYRRIYDLIELCGSLH | EBV EBNA 3C | HLA-B27 |
| | (SEQ ID NO: 19) | | |
| 20v | AQGMAYPLHEQHGMAPCPV | EBV EBNA 3A | IILA-B35 |
| | (SEQ ID NO: 20) | | |
| 21v | NLLQTEENLLDFVRFMGVMS | EBV EBNA-3C | HLA-B44 |
| | (SEQ ID NO: 21) | | |
| 22v | NLKYQEFFWDANDIYRIFAE | CMV pp65 | HLA-B44 |
| | (SEQ ID NO: 22) | | |
| 23v | TTERKTPRVTGGGAMAGAST | CMV pp65 | HLA-B7 |
| | (SEQ ID NO: 23) | | |
| 24v | KNILMQYIKANSKFIGITELKKLES | Tetanus Toxin | HLA-Klasse II Allele |
| | (SEQ ID NO: 24) | Precursor | not determined |
| 25v | EYNDMFNNFTVSFWLRVPKVSASHLEQYGTN | Tetanus Toxin | HLA-DRB1 HLA-DPB1 |
| | (SEQ ID NO: 25) | Precursor | |
| 26v | LYNGLKFIIKRYTPNNEIDSFVKSGD | Tetanus Toxin | HLA-DRB1 |
| | (SEQ ID NO: 26) | Precursor | |
| 27v | NNNQYVSIDKFRIFCKALNPKEIEKL | Tetanus Toxin | HLA-DRB1 |
| | (SEQ ID NO: 27) | Precursor | |

In a preferred embodiment, the peptide pool consists or comprises epitopes enabled to activate all kinds of T-cells, preferably, T helper cells and cytotoxic T cells, more preferably at least CD8 memory and CD4 memory cells.

In another preferred embodiment the first substance additionally comprises a substance capable to stimulate NKT (natural killer T cells), preferably alpha-Galactosylceramide (a-Gal-Cer). In a further preferred embodiment, alpha-Galactosylceramide (a-Gal-Cer) is a synthetic a-Gal-Cer analogue, preferably KRN7000 or the a-Gal-Cer analogue 8. Preferably the concentration of alpha-Galactosylceramide (a-Gal-Cer) is ranging from 0.0001 μg/ml to 1000 g/ml, more preferably from 0.001 μg/ml to 100 μg/ml, even more preferably from 0.01 μg/ml to 10 μg/ml, most preferably from 6.5 μg/ml to 10 μg/ml. In another preferred embodiment the concentration of alpha-Galactosylceramide is 0.001, 0.001, 0.01, 0.5, 1, 2, 3, 4, 5, 6, 7, 8, 9, 10, 11, 12, 13, 14, 15, 16, 17, 18, 19, 20, 25, 30, 35, 40, 50, 60, 70, 80, 90 or 100 μg/ml, most preferably 6.5 μg/ml or 10 μg/ml.

In a preferred embodiment, the composition according to the present invention comprises a peptide pool, LPS, polyinosinic:polycytidylic acid (poly(I:C)), IL-12 and a-Gal-Cer, preferably KRN7000 or the a-Gal-Cer analogue 8. In a more preferred embodiment, the composition according to the present invention comprises 1 μg p.p./ml peptide pool, 1 EU/ml LPS, 10 μg/ml poly(I:C), 0.01 μg/ml IL-12 and 10 μg/ml a-Gal-Cer preferably KRN7000 or the a-Gal-Cer analogue 8, even more preferred 1 μg p.p./ml CEFTv pool, 1 EU/ml LPS, 10 μg/ml poly(I:C), 0.01 μg/ml IL-12 and 10 μg/ml a-Gal-Cer, preferably KRN7000 or the a-Gal-Cer analogue 8. In another preferred embodiment, the composition according to the present invention comprises 1 μg p.p./ml peptide pool, 1 EU/ml LPS, 10 μg/ml poly(I:C), 0.01 μg/ml IL-12 and 6.5 μg/ml a-Gal-Cer preferably KRN7000 or the a-Gal-Cer analogue 8, even more preferred 1 μg p.p./ml CEFTv pool, 1 EU/ml LPS, 10 μg/ml poly(I:C), 0.01 μg/ml IL-12 and 6.5 μg/ml a-Gal-Cer, preferably KRN7000 or the a-Gal-Cer analogue 8.

The second substance according to the present invention may stimulate NK cells to produce at least one immune effector molecule. The capability of the second substance to stimulate NK cells to produce at least one immune effector molecule may be determined by determining the immune effector molecule(s), preferably cytokines, more preferably IFN-β, INF-γ, TNF-α, IL-2, IL-4, IL-5, IL-6, IL-10, IL-12, IL-13, GM-CSF, TGF-β, MIP1a, MIP1b, 4-1BB, preferably soluble and/or membrane bound, CD25, perforin and/or granzyme by means of an ELISA, an ELISpot assay or by means of FACS technology, PCR, quantitative PCR (qPCR), multiplex bead assays and/or microarray. In a preferred embodiment the soluble immune effector molecules, preferably cytokines, more preferably IFN-β, INF-γ, TNF-α, IL-2, IL-4, IL-5, IL-6, IL-10, IL-12, IL-13, GM-CSF, TGF-β, MIP1a, MIP1b, 4-1BB, preferably soluble may be localized intracellular and/or extracellular.

In a preferred embodiment the composition according to the present invention comprises at least one, preferably two or more second substances, most preferably 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90 or 100 second substances.

In another embodiment, the second substance according to the present invention comprises a double stranded nucleic acid, single stranded nucleic acid, unmethylated CpG oligodeoxynucleotide, TLR agonist except lipopolysaccharide (LPS), arabinoxylan (BioBran® MGN-3), an immunoglobulin, a murine Cytomegalovirus (MCMV)-encoded protein, CCL5 (chemokine (C—C motif) ligand 5), a UL-16-binding protein (ULBP), CD48, CD70, CD155, CD112, Ned-1, B7-H6, ICAM-1, RAE-1 (retinoic acid early inducible 1), H60, Multi and hemagglutinin. Preferably, the murine Cytomegalovirus-encoded protein may be m157. Preferably, the ULBP may be ULBP1, ULBP2, ULBP3, ULBP4, ULBP5 or ULBP6. Preferably, the immunoglobulin may be IgG. Preferably the hemagglutinin may be a viral hemagglutinin, preferably an influenza hemagglutinin. Preferably, the second substance of the composition according to the present invention comprises one or more TLR (Toll-like receptor) agonists. In another embodiment of the present invention, the one or more TLR agonists may be a TLR-7/8, TLR-4, TLR-3 or TLR-2 agonist. Examples include the imidazoquinoline, R848, lipomannan, polyinosinic:polycytidylic acid (poly(I:C)), poly(I:C)-LMW (low molecular weight), poly(I:C)-LMW/LyoVec, Pam3CS 4 and CpG oligodeoxynucleotides. In one embodiment, the TLR agonist is poly(I:C), poly(I:C)-LMW or poly(I:C)-LMW/LyoVec.

In another embodiment of the present invention the second substance comprise an extract of Viscum album (mistletoe), Cichorium intybus, Echinaces purpurae root, Derris scandens, Nigella sativum seeds, Allium sativum bulb, Onopordum acanthium stem and leaves, Allium cepa bulbs, Chinese herb (e.g. Shikaron), Phyllantus emblica, and mushrooms, like Lentinus edodes and Agaricus blazei.

In a preferred embodiment, the composition according to the present invention comprises LPS and/or urea each in a concentration which is not capable to stimulate immune cells to release an immune effector molecule, preferably IFN-γ if applied alone. Preferably, the concentration of urea is 10, 20, 30, 40, 50, 100, 150, 200, 300, 400, 500, 600, 700, 800, 900 or 1000 mM and/or the concentration of LPS is 0.5, 0.75, 1, 1.5, 2, 2.5, 3, 4, 5, 6, 7, 8, 9, 10, 11, 12, 13, 14, 15, 16, 15, 16, 17, 18, 19 or 20 EU/ml, more preferably the concentration of urea is 100 mM and/or the concentration of LPS is 1 EU/ml.

In a preferred embodiment, the composition according to the present invention comprises LPS and/or urea each in a concentration which is capable to stimulate immune cells, preferably monocytes to release an immune effector molecule, preferably TNF-α and/or IL-10 if applied alone. Preferably, the concentration of urea is 10, 20, 30, 40, 50, 100, 150, 200, 300, 400, 500, 600, 700, 800, 900 or 1000 mM and/or the concentration of LPS is 0.5, 0.75, 1, 1.5, 2, 2.5, 3, 4, 5, 6, 7, 8, 9, 10, 11, 12, 13, 14, 15, 16, 15, 16, 17, 18, 19 or 20 EU/ml, more preferably the concentration of urea is 100 mM and/or the concentration of LPS is 1 EU/ml.

In another preferred embodiment the concentration of LPS is ranging from 0.001 EU/ml to 1000 EU/ml, more preferably from 0.01 EU/ml to 100 EU/ml, even more preferably from 0.1 EU/ml to 20 EU/ml and most preferably the concentration is 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 EU/ml, most preferably 1 EU/ml.

In a further preferred embodiment LPS according to the present invention is a bacterial lipopolysaccharide, preferably lipopolysaccharide from Escherichia coli 026:B6. In another preferred embodiment LPS according to the present invention is the lipopolysaccharide, according to the catalogue number: L4391 as provided by the company Sigma-Aldrich.

In another preferred embodiment the concentration of LPS is verified using the Limulus Amebocyte Lysate (LAL) assay as provided by Lonza Group Ltd. Basel, Schweiz. DEML

In another preferred embodiment the concentration of urea is ranging from 0.001 mM to 1000 mM, more preferably from 0.01 mM to 500 mM, even more preferably from 0.1 mM to 100 mM and most preferably the concentration is 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190, 200, 300, 400, 500, 600, 700, 800, 900 or 1000 mM. In another preferred embodiment the concentration of urea is 100 mM. Urea may be important, because it is capable to solve an interaction between LPS and a peptide, protein or polypeptide.

In another preferred embodiment the concentration of the peptide pool, preferably CEFTv is ranging from 0.00001 μg p.p./ml to 1000 μg p.p./ml, more preferably from 0.0001 μg p.p./ml to 100 μg p.p./ml, even more preferably from 0.01 μg p.p./ml to 10 μg p.p./ml and most preferably the concentration is 0.05, 0.1, 0.2, 0.3, 0.4, 0.5, 0.6, 0.7, 0.8, 0.9 or 1 μg p.p./ml, even more preferably 1 μg p.p./ml.

In another preferred embodiment the concentration of the anti-CD3 antibody is ranging from 1 ng/10⁸ cells to 500 μg/10⁸ cells, more preferably from 5 ng/10⁸ cells to 100 μg/10⁸ cells, even more preferably from 5 ng/10⁸ cells to 100 μg/10⁸ cells and most preferably the concentration is 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9 or 10 μg/10⁸ cells, even more preferably 0.76 μg/10⁸ cells or 30 ng/10⁸ cells.

In another preferred embodiment the concentration of the anti-CD28 antibody is ranging from 1 ng/10⁸ cells to 5 mg/10⁸ cells, more preferably from 10 ng/10⁸ cells to 1 mg/10⁸ cells, even more preferably from 100 ng/10⁸ cells to 100 μg/10⁸ cells and most preferably the concentration is 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13 14 or 15 μg/10⁸ cells, even more preferably 3.8 μg/10⁸ cells or 12 μg/10⁸ cells.

In another preferred embodiment the concentration of poly(I:C), poly(I:C)-LMW or poly(I:C)-LMW/LyoVec is ranging from 0.0001 μg/ml to 1000 g/ml, more preferably from 0.001 μg/ml to 100 μg/ml, even more preferably from 0.01 μg/ml to 10 μg/ml. In another preferred embodiment the concentration of poly(I:C), poly(I:C)-LMW or poly(I:C)-LMW/LyoVec is 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190 or 200 μg/ml, even more preferably 10 μg/ml.

It becomes clear that the composition according to the present invention comprises LPS and/or urea and additionally at least one first substance and at least one second substance. Moreover, it should be understood that there is no overlap in the single components of the composition, i.e. the composition according to the present invention comprises at least three different components including LPS and/or urea. In other words, the first and second substance(s) are not LPS or urea.

In a further embodiment, the composition according to the present invention may comprise an additional agent modulating the activity of regulatory T-cells (T-reg cells). The latter encompasses inhibiting the suppressor function of T-reg cells. Agents which modulate T-reg cells encompassed herein include a CD25 ligand; a sense or antisense oligonucleotide to genetic material encoding JAK1 or TYK2; a neutralizing antibody; a CpG containing oligonucleotide; an oligonucleotide acting as a toll-like receptor (TLR) modulating agent; and other TLR modulating agents.

Examples of inhibitors or modulators of T-reg function include CD25 ligands such as but not limited to a polyclonal or monoclonal antibody to CD25 or an antigen-binding fragment thereof, humanized or deimmunized polyclonal or monoclonal antibodies to CD25 or a recombinant or synthetic form of the polyclonal or monoclonal antibodies. Other examples of agents include sense or antisense nucleic and molecules directed to the mRNA or DNA (i.e. genetic material) encoding Janus Tyrosine Kinase 1 (JAK1) or Tyrosine Kinase 2 (TYK2) or small molecule inhibitors of JAK1 or TYK2 proteins. Reference to “small molecules” includes immunoglobulin new antigen receptors (IgNARs) as described in WO 2005/1 18629. Further examples of suitable agents stimulating agents such as CpG molecules which act via Toll-like receptors (TLRs) and/or other mechanisms. Hence, CpG containing oligonucleotides and an oligonucleotide acting as a TLR modulating agent also form part of the present disclosure. A CpG molecule is an oligonucleotide comprising a CpG sequence or motif.

A single type of agent may be used or two or more types of agents may be employed to modulate T-reg cells. For example, the assay may be conducted with a CD25 ligand and a JAK1/TYK2 sense or antisense oligonucleotide; a CD25 ligand and a TLR modulating agent; a JAK1/TYK2 sense or antisense oligonucleotide and a TLR modulating agent; or a CD25 ligand, a JAK1/TY 2 sense or antisense oligonucleotide and a TLR modulating agent. Alternatively, just one type of agent is employed. In another alternative, a CpG comprising oligonucleotide and a TLR modulating agent is used.

In a further embodiment, the composition according to the present invention comprises additionally polypeptides comprising 15, 16, 17, 18, 19, 20, 21, 22, 23, 24, 25, 26, 27, 28, 29, 30, 40, 50, 60, 70, 80, 90, 100 or more amino acids. These polypeptides are processed by the antigen presenting cells (APC) and cannot be loaded via MHC class 1 without processing. Subjects which are immunosuppressed may not process the polypeptides and thus, APC may not induce T cells which produce immune effector cells.

In a further embodiment, the composition according to the present invention comprises a mixture of urea and cyanate. In another embodiment, the first substance according to the invention may be pretreated with urea, cyanate ions or a mixture of urea and cyanate. The pre-treatment with cyanate ions may be performed in accordance with the teaching of WO2010/115984.

In a further embodiment, the composition according to the present invention additionally comprises an enhancer for increasing of the stimulatory activity of the first and/or second substance on immune cells. In another embodiment, the composition according to the present invention additionally comprises an enhancer for increasing the stimulatory activity of poly(I:C) on immune cells. In a preferred embodiment the enhancer is IL-2, IL-12, IL-15 and/or IL-18, more preferably IL-12. In a preferred embodiment the enhancer is one or more accessory cells, e.g. dendritic cells. In another preferred embodiment the concentration of IL-2, IL-12, IL-15 and/or IL-18 is ranging from 0.001 μg/ml to 100 μg/ml, more preferably from 0.01 μg/ml to 10 μg/ml, most preferably the concentration is 0.0001, 0.0005, 0.001, 0.005, 0.01, 0.05, 0.1, 0.2, 0.3, 0.4, 0.5, 1, 2, 3, 4 or 5 μg/ml, even more preferably 0.01 μg/ml.

In a preferred embodiment the composition according to the present invention comprises a first substance, preferably a peptide pool, a poly(I:C) variant, preferably poly(I:C), poly(I:C)-LMW or poly(I:C)-LMW/LyoVec, LPS and urea. In a preferred embodiment the composition according to the present invention comprises a first substance, preferably a peptide pool, a poly(I:C) variant, preferably poly(I:C), poly(I:C)-LMW or poly(I:C)-LMW/LyoVec, LPS and IL-12. In a further preferred embodiment the composition according to the present invention comprises a first substance, preferably a peptide pool, a poly(I:C) variant, preferably poly(I:C), poly(I:C)-LMW or poly(I:C)-LMW/LyoVec, LPS, IL-12 and urea. In a preferred embodiment the composition according to the present invention comprises a first substance, preferably a peptide pool, a poly(I:C) variant, preferably poly(I:C), poly(I:C)-LMW or poly(I:C)-LMW/LyoVec and urea. In a preferred embodiment the composition according to the present invention comprises a first substance, preferably a peptide pool, a poly(I:C) variant, preferably poly(I:C), poly(I:C)-LMW or poly(I:C)-LMW/LyoVec and IL-12. In a preferred embodiment the composition according to the present invention comprises a first substance, preferably a peptide pool, LPS and urea. In a further preferred embodiment the composition according to the present invention comprises a first substance, preferably a peptide pool, LPS, a poly(I:C) variant, preferably poly(I:C), poly(I:C)-LMW or poly(I:C)-LMW/LyoVec.

In a preferred embodiment the composition according to the present invention consists of a first substance, preferably a peptide pool, a poly(I:C) variant, preferably poly(I:C), poly(I:C)-LMW or poly(I:C)-LMW/LyoVec, LPS and urea. In a preferred embodiment the composition according to the present invention consists of a first substance, preferably a peptide pool, a poly(I:C) variant, preferably poly(I:C), poly(I:C)-LMW or poly(I:C)-LMW/LyoVec, LPS and IL-12. In a further preferred embodiment the composition according to the present invention consists of a first substance, preferably a peptide pool, a poly(I:C) variant, preferably poly(I:C), poly(I:C)-LMW or poly(I:C)-LMW/LyoVec, LPS, IL-12 and urea. In a preferred embodiment the composition according to the present invention consists of a first substance, preferably a peptide pool, a poly(I:C) variant, preferably poly(I:C), poly(I:C)-LMW or poly(I:C)-LMW/LyoVec and urea. In a preferred embodiment the composition according to the present invention consists of a first substance, preferably a peptide pool, a poly(I:C) variant, preferably poly(I:C), poly(I:C)-LMW or poly(I:C)-LMW/LyoVec and IL-12. In a preferred embodiment the composition according to the present invention consists of a first substance, preferably a peptide pool, LPS and urea. In a further preferred embodiment the composition according to the present invention consists of a first substance, preferably a peptide pool, LPS, a poly(I:C) variant, preferably poly(I:C), poly(I:C)-LMW or poly(I:C)-LMW/LyoVec.

In a preferred embodiment the composition according to the present invention comprises a first substance, preferably an antibody or a combination of two antibodies, preferably anti-CD3 and/or anti-CD28, a poly(I:C) variant, preferably poly(I:C), poly(I:C)-LMW or poly(I:C)-LMW/LyoVec, LPS and urea. In a preferred embodiment the composition according to the present invention comprises a first substance, preferably an antibody or a combination of two antibodies, preferably anti-CD3 and/or anti-CD28, a poly(I:C) variant, preferably poly(I:C), poly(I:C)-LMW or poly(I:C)-LMW/LyoVec, LPS and IL-12. In a further preferred embodiment the composition according to the present invention comprises a first substance, preferably an antibody or a combination of two antibodies, preferably anti-CD3 and/or anti-CD28, a poly(I:C) variant, preferably poly(I:C), poly(I:C)-LMW or poly(I:C)-LMW/LyoVec, LPS, IL-12 and urea. In a preferred embodiment the composition according to the present invention comprises a first substance, preferably an antibody or a combination of two antibodies, preferably anti-CD3 and/or anti-CD28, a poly(I:C) variant, preferably poly(I:C), poly(I:C)-LMW or poly(I:C)-LMW/LyoVec and urea. In a preferred embodiment the composition according to the present invention comprises a first substance, preferably an antibody or a combination of two antibodies, preferably anti-CD3 and/or anti-CD28, a poly(I:C) variant, preferably poly(I:C), poly(I:C)-LMW or poly(I:C)-LMW/LyoVec and IL-12. In a preferred embodiment the composition according to the present invention comprises a first substance, preferably an antibody or a combination of two antibodies, preferably anti-CD3 and/or anti-CD28, LPS and urea. In a further preferred embodiment the composition according to the present invention comprises a first substance, preferably an antibody or a combination of two antibodies, preferably anti-CD3 and/or anti-CD28, LPS, a poly(I:C) variant, preferably poly(I:C), poly(I:C)-LMW or poly(I:C)-LMW/LyoVec.

In a preferred embodiment the composition according to the present invention consists of a first substance, preferably an antibody or a combination of two antibodies, preferably anti-CD3 and/or anti-CD28, a poly(I:C) variant, preferably poly(I:C), poly(I:C)-LMW or poly(I:C)-LMW/LyoVec, LPS and urea. In a preferred embodiment the composition according to the present invention consists of a first substance, preferably an antibody or a combination of two antibodies, preferably anti-CD3 and/or anti-CD28 a poly(I:C) variant, preferably poly(I:C), poly(I:C)-LMW or poly(I:C)-LMW/LyoVec, LPS and IL-12. In a further preferred embodiment the composition according to the present invention consists of a first substance, preferably an antibody or a combination of two antibodies, preferably anti-CD3 and/or anti-CD28, a poly(I:C) variant, preferably poly(I:C), poly(I:C)-LMW or poly(I:C)-LMW/LyoVec, LPS, IL-12 and urea. In a preferred embodiment the composition according to the present invention consists of a first substance, preferably an antibody or a combination of two antibodies, preferably anti-CD3 and/or anti-CD28, a poly(I:C) variant, preferably poly(I:C), poly(I:C)-LMW or poly(I:C)-LMW/LyoVec and urea. In a preferred embodiment the composition according to the present invention consists of a first substance, preferably an antibody or a combination of two antibodies, preferably anti-CD3 and/or anti-CD28, a poly(I:C) variant, preferably poly(I:C), poly(I:C)-LMW or poly(I:C)-LMW/LyoVec and IL-12. In a preferred embodiment the composition according to the present invention consists of a first substance, preferably an antibody or a combination of two antibodies, preferably anti-CD3 and/or anti-CD28, LPS and urea. In a further preferred embodiment the composition according to the present invention consists of a first substance, preferably an antibody or a combination of two antibodies, preferably anti-CD3 and/or anti-CD28, LPS, a poly(I:C) variant, preferably poly(I:C), poly(I:C)-LMW or poly(I:C)-LMW/LyoVec.

The composition according to the present invention may comprise further compounds comprising suitable carriers, stabilizers, buffers, medium or other suitable reagents.

In a preferred embodiment the composition according to the present invention comprises a peptide pool, preferably CEFTv in a concentration ranging from 0.00001 μg p.p./ml to 1000 μg p.p./ml, more preferably from 0.0001 μg p.p./ml to 100 μg p.p./ml, even more preferably from 0.01 μg p.p./ml to 10 μg p.p./ml, LPS in a concentration ranging from 0.001 EU/ml to 1000 EU/ml, more preferably from 0.01 EU/ml to 100 EU/ml, even more preferably from 0.1 EU/ml to 20 EU/ml and a poly(I:C) variant, preferably poly(I:C), poly(I:C)-LMW or poly(I:C)-LMW/LyoVec in a concentration ranging from 0.0001 μg/ml to 1000 g/ml, more preferably from 0.001 μg/ml to 100 μg/ml, even more preferably from 0.01 μg/ml to 10 μg/ml.

In a preferred embodiment the composition according to the present invention comprises an antibody or a combination of two antibodies, preferably anti-CD3 and/or anti-CD28 in a concentration ranging from 1 ng/10⁸ cells to 5 mg/10⁸ cells, more preferably from 10 ng/10⁸ cells to 1 mg/10⁸ cells, even more preferably from 100 ng/10⁸ cells to 100 μg/10⁸ cells, LPS in a concentration ranging from 0.001 EU/ml to 1000 EU/ml, more preferably from 0.01 EU/ml to 100 EU/ml, even more preferably from 0.1 EU/ml to 20 EU/ml and a poly(I:C) variant, preferably poly(I:C), poly(I:C)-LMW or poly(I:C)-LMW/LyoVec in a concentration ranging from 0.0001 μg/ml to 1000 g/ml, more preferably from 0.001 μg/ml to 100 μg/ml, even more preferably from 0.01 μg/ml to 10 μg/ml.

In a further preferred embodiment the composition according to the present invention comprises a peptide pool, preferably CEFTv in a concentration ranging from 0.00001 μg p.p./ml to 1000 μg p.p./ml, more preferably from 0.0001 μg p.p./ml to 100 μg p.p./ml, even more preferably from 0.01 μg p.p./ml to 10 μg p.p./ml, an enhancer, preferably IL-12 in a concentration ranging from 0.001 μg/ml to 100 μg/ml, more preferably from 0.01 μg/ml to 10 μg/ml, LPS in a concentration ranging from 0.001 EU/ml to 1000 EU/ml, more preferably from 0.01 EU/ml to 100 EU/ml, even more preferably from 0.1 EU/ml to 20 EU/ml and a poly(I:C) variant, preferably poly(I:C), poly(I:C)-LMW or poly(I:C)-LMW/LyoVec in a concentration ranging from 0.0001 μg/ml to 1000 g/ml, more preferably from 0.001 μg/ml to 100 μg/ml, even more preferably from 0.01 μg/ml to 10 μg/ml.

In a further preferred embodiment the composition according to the present invention comprises an antibody or a combination of two antibodies, preferably anti-CD3 and/or anti-CD28 in a concentration ranging from 1 ng/10⁸ cells to 5 mg/10⁸ cells, more preferably from 10 ng/10⁸ cells to 1 mg/10⁸ cells, even more preferably from 100 ng/10⁸ cells to 100 μg/10⁸ cells, an enhancer, preferably IL-12 in a concentration ranging from 0.001 μg/ml to 100 μg/ml, more preferably from 0.01 μg/ml to 10 μg/ml, LPS in a concentration ranging from 0.001 EU/ml to 1000 EU/ml, more preferably from 0.01 EU/ml to 100 EU/ml, even more preferably from 0.1 EU/ml to 20 EU/ml and a poly(I:C) variant, preferably poly(I:C), poly(I:C)-LMW or poly(I:C)-LMW/LyoVec in a concentration ranging from 0.0001 μg/ml to 1000 g/ml, more preferably from 0.001 μg/ml to 100 μg/ml, even more preferably from 0.01 μg/ml to 10 μg/ml.

In a further preferred embodiment the composition according to the present invention comprises a peptide pool, preferably CEFTv in a concentration ranging from 0.00001 μg p.p./ml to 1000 μg p.p./ml, more preferably from 0.0001 μg p.p./ml to 100 μg p.p./ml, even more preferably from 0.01 μg p.p./ml to 10 μg p.p./ml, LPS in a concentration ranging from 0.001 EU/ml to 1000 EU/ml, more preferably from 0.01 EU/ml to 100 EU/ml, even more preferably from 0.1 EU/ml to 20 EU/ml, a poly(I:C) variant, preferably poly(I:C), poly(I:C)-LMW or poly(I:C)-LMW/LyoVec in a concentration ranging from 0.0001 μg/ml to 1000 g/ml, more preferably from 0.001 μg/ml to 100 μg/ml, even more preferably from 0.01 μg/ml to 10 μg/ml and urea in a concentration ranging from 0.001 mM to 1000 mM, more preferably from 0.01 mM to 500 mM, even more preferably from 0.1 mM to 100 mM.

In a further preferred embodiment the composition according to the present invention comprises an antibody or a combination of two antibodies, preferably anti-CD3 and/or anti-CD28 in a concentration ranging from 1 ng/10⁸ cells to 5 mg/10⁸ cells, more preferably from 10 ng/10⁸ cells to 1 mg/10⁸ cells, even more preferably from 100 ng/10⁸ cells to 100 μg/10⁸ cells, LPS in a concentration ranging from 0.001 EU/ml to 1000 EU/ml, more preferably from 0.01 EU/ml to 100 EU/ml, even more preferably from 0.1 EU/ml to 20 EU/ml, a poly(I:C) variant, preferably poly(I:C), poly(I:C)-LMW or poly(I:C)-LMW/LyoVec in a concentration ranging from 0.0001 μg/ml to 1000 g/ml, more preferably from 0.001 μg/ml to 100 μg/ml, even more preferably from 0.01 μg/ml to 10 μg/ml and urea in a concentration ranging from 0.001 mM to 1000 mM, more preferably from 0.01 mM to 500 mM, even more preferably from 0.1 mM to 100 mM.

In a further preferred embodiment the composition according to the present invention comprises peptide pool, preferably CEFTv in a concentration ranging from 0.00001 μg p.p./ml to 1000 μg p.p./ml, more preferably from 0.0001 μg p.p./ml to 100 μg p.p./ml, even more preferably from 0.01 μg p.p./ml to 10 μg p.p./ml, LPS in a concentration ranging from 0.001 EU/ml to 1000 EU/ml, more preferably from 0.01 EU/ml to 100 EU/ml, even more preferably from 0.1 EU/ml to 20 EU/ml, a poly(I:C) variant, preferably poly(I:C), poly(I:C)-LMW or poly(I:C)-LMW/LyoVec in a concentration ranging from 0.0001 μg/ml to 1000 g/ml, more preferably from 0.001 μg/ml to 100 μg/ml, even more preferably from 0.01 μg/ml to 10 μg/ml, IL-12 in a concentration ranging from 0.001 μg/ml to 100 μg/ml, more preferably from 0.01 μg/ml to 10 μg/ml and urea in a concentration ranging from 0.001 mM to 1000 mM, more preferably from 0.01 mM to 500 mM, even more preferably from 0.1 mM to 100 mM.

In a further preferred embodiment the composition according to the present invention comprises an antibody or a combination of two antibodies, preferably anti-CD3 and/or anti-CD28 in a concentration ranging from 1 ng/10⁸ cells to 5 mg/10⁸ cells, more preferably from 10 ng/10⁸ cells to 1 mg/10⁸ cells, even more preferably from 100 ng/10⁸ cells to 100 μg/10⁸ cells, LPS in a concentration ranging from 0.001 EU/ml to 1000 EU/ml, more preferably from 0.01 EU/ml to 100 EU/ml, even more preferably from 0.1 EU/ml to 20 EU/ml, a poly(I:C) variant, preferably poly(I:C), poly(I:C)-LMW or poly(I:C)-LMW/LyoVec in a concentration ranging from 0.0001 μg/ml to 1000 g/ml, more preferably from 0.001 μg/ml to 100 μg/ml, even more preferably from 0.01 μg/ml to 10 μg/ml, IL-12 in a concentration ranging from 0.001 μg/ml to 100 μg/ml, more preferably from 0.01 μg/ml to 10 μg/ml and urea in a concentration ranging from 0.001 mM to 1000 mM, more preferably from 0.01 mM to 500 mM, even more preferably from 0.1 mM to 100 mM.

In a further preferred embodiment the composition according to the present invention comprises a peptide pool, preferably CEFTv in a concentration ranging from 0.00001 μg p.p./ml to 1000 μg p.p./ml, more preferably from 0.0001 μg p.p./ml to 100 μg p.p./ml, even more preferably from 0.01 μg p.p./ml to 10 μg p.p./ml, a poly(I:C) variant, preferably poly(I:C), poly(I:C)-LMW or poly(I:C)-LMW/LyoVec in a concentration ranging from 0.0001 μg/ml to 1000 g/ml, more preferably from 0.001 μg/ml to 100 μg/ml, even more preferably from 0.01 μg/ml to 10 μg/ml and urea in a concentration ranging from 0.001 mM to 1000 mM, more preferably from 0.01 mM to 500 mM, even more preferably from 0.1 mM to 100 mM.

In a further preferred embodiment the composition according to the present invention comprises an antibody or a combination of two antibodies, preferably anti-CD3 and/or anti-CD28 in a concentration ranging from 1 ng/10⁸ cells to 5 mg/10⁸ cells, more preferably from 10 ng/10⁸ cells to 1 mg/10⁸ cells, even more preferably from 100 ng/10⁸ cells to 100 μg/10⁸ cells, a poly(I:C) variant, preferably poly(I:C), poly(I:C)-LMW or poly(I:C)-LMW/LyoVec in a concentration ranging from 0.0001 μg/ml to 1000 g/ml, more preferably from 0.001 μg/ml to 100 μg/ml, even more preferably from 0.01 μg/ml to 10 μg/ml and urea in a concentration ranging from 0.001 mM to 1000 mM, more preferably from 0.01 mM to 500 mM, even more preferably from 0.1 mM to 100 mM.

In a further preferred embodiment the composition according to the present invention comprises a peptide pool, preferably CEFTv in a concentration ranging from 0.00001 μg p.p./ml to 1000 μg p.p./ml, more preferably from 0.0001 μg p.p./ml to 100 μg p.p./ml, even more preferably from 0.01 μg p.p./ml to 10 μg p.p./ml, LPS in a concentration ranging from 0.001 EU/ml to 1000 EU/ml, more preferably from 0.01 EU/ml to 100 EU/ml, even more preferably from 0.1 EU/ml to 20 EU/ml, and urea in a concentration ranging from 0.001 mM to 1000 mM, more preferably from 0.01 mM to 500 mM, even more preferably from 0.1 mM to 100 mM.

In a further preferred embodiment the composition according to the present invention comprises an antibody or a combination of two antibodies, preferably anti-CD3 and/or anti-CD28 in a concentration ranging from 1 ng/10⁸ cells to 5 mg/10⁸ cells, more preferably from 10 ng/10⁸ cells to 1 mg/10⁸ cells, even more preferably from 100 ng/10⁸ cells to 100 μg/10⁸ cells, LPS in a concentration ranging from 0.001 EU/ml to 1000 EU/ml, more preferably from 0.01 EU/ml to 100 EU/ml, even more preferably from 0.1 EU/ml to 20 EU/ml, and urea in a concentration ranging from 0.001 mM to 1000 mM, more preferably from 0.01 mM to 500 mM, even more preferably from 0.1 mM to 100 mM.

In a further preferred embodiment the composition according to the present invention comprises a peptide pool, preferably CEFTv in a concentration ranging from 0.00001 μg p.p./ml to 1000 μg p.p./ml, more preferably from 0.0001 μg p.p./ml to 100 μg p.p./ml, even more preferably from 0.01 μg p.p./ml to 10 μg p.p./ml, a poly(I:C) variant, preferably poly(I:C), poly(I:C)-LMW or poly(I:C)-LMW/LyoVec in a concentration ranging from 0.0001 μg/ml to 1000 g/ml, more preferably from 0.001 μg/ml to 100 μg/ml, even more preferably from 0.01 μg/ml to 10 μg/ml and IL-12 in a concentration ranging from 0.001 μg/ml to 100 μg/ml, more preferably from 0.01 μg/ml to 10 μg/ml.

In a further preferred embodiment the composition according to the present invention comprises an antibody or a combination of two antibodies, preferably anti-CD3 and/or anti-CD28 in a concentration ranging from 1 ng/10⁸ cells to 5 mg/10⁸ cells, more preferably from 10 ng/10⁸ cells to 1 mg/10⁸ cells, even more preferably from 100 ng/10⁸ cells to 100 μg/10⁸ cells, a poly(I:C) variant, preferably poly(I:C), poly(I:C)-LMW or poly(I:C)-LMW/LyoVec in a concentration ranging from 0.0001 μg/ml to 1000 g/ml, more preferably from 0.001 μg/ml to 100 μg/ml, even more preferably from 0.01 μg/ml to 10 μg/ml and IL-12 in a concentration ranging from 0.001 μg/ml to 100 μg/ml, more preferably from 0.01 μg/ml to 10 μg/ml.

In a preferred embodiment the composition according to the present invention comprises 0.05, 0.1, 0.2, 0.3, 0.4, 0.5, 0.6, 0.7, 0.8, 0.9 or 1 μg p.p./ml peptide pool, preferably CEFTv, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 EU/ml LPS and 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190 or 200 μg/ml of a poly(I:C) variant, preferably poly(I:C), poly(I:C)-LMW or poly(I:C)-LMW/LyoVec.

In a further preferred embodiment the composition according to the present invention comprises 0.05, 0.1, 0.2, 0.3, 0.4, 0.5, 0.6, 0.7, 0.8, 0.9 or 1 μg p.p./ml peptide pool, preferably CEFTv, 0.0001, 0.0005, 0.001, 0.005, 0.01, 0.05, 0.1, 0.2, 0.3, 0.4, 0.5, 1, 2, 3, 4 or 5 μg/ml IL-12, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 EU/ml LPS and 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190 or 200 μg/ml of a poly(I:C) variant, preferably poly(I:C), poly(I:C)-LMW or poly(I:C)-LMW/LyoVec.

In a further preferred embodiment the composition according to the present invention comprises 0.05, 0.1, 0.2, 0.3, 0.4, 0.5, 0.6, 0.7, 0.8, 0.9 or 1 μg p.p./ml peptide pool, preferably CEFTv, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 EU/ml LPS, 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190 or 200 μg/ml of a poly(I:C) variant, preferably poly(I:C), poly(I:C)-LMW or poly(I:C)-LMW/LyoVec and 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190, 200, 300, 400, 500, 600, 700, 800, 900 or 1000 mM urea.

In a further preferred embodiment the composition according to the present invention comprises 0.05, 0.1, 0.2, 0.3, 0.4, 0.5, 0.6, 0.7, 0.8, 0.9 or 1 μg p.p./ml peptide pool, preferably CEFTv, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 EU/ml LPS, 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190 or 200 μg/ml of a poly(I:C) variant, preferably poly(I:C), poly(I:C)-LMW or poly(I:C)-LMW/LyoVec, 0.0001, 0.0005, 0.001, 0.005, 0.01, 0.05, 0.1, 0.2, 0.3, 0.4, 0.5, 1, 2, 3, 4 or 5 μg/ml IL-12 and 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190, 200, 300, 400, 500 600, 700, 800, 900 or 1000 mM urea.

In a further preferred embodiment the composition according to the present invention comprises 0.05, 0.1, 0.2, 0.3, 0.4, 0.5, 0.6, 0.7, 0.8, 0.9 or 1 μg p.p./ml peptide pool, preferably CEFTv, 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190 or 200 μg/ml of a poly(I:C) variant, preferably poly(I:C), poly(I:C)-LMW or poly(I:C)-LMW/LyoVec, and 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190, 200, 300, 400, 500 600, 700, 800, 900 or 1000 mM urea.

In a further preferred embodiment the composition according to the present invention comprises 0.05, 0.1, 0.2, 0.3, 0.4, 0.5, 0.6, 0.7, 0.8, 0.9 or 1 μg p.p./ml peptide pool, preferably CEFTv, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 EU/ml LPS, and 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190, 200, 300, 400, 500, 600, 700, 800, 900 or 1000 mM urea.

In a further preferred embodiment the composition according to the present invention comprises 0.05, 0.1, 0.2, 0.3, 0.4, 0.5, 0.6, 0.7, 0.8, 0.9 or 1 μg p.p./ml peptide pool, preferably CEFTv, 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190 or 200 μg/ml of a poly(I:C) variant, preferably poly(I:C), poly(I:C)-LMW or poly(I:C)-LMW/LyoVec and 0.0001, 0.0005, 0.001, 0.005, 0.01, 0.05, 0.1, 0.2, 0.3, 0.4, 0.5, 1, 2, 3, 4 or 5 μg/ml IL-12.

In a preferred embodiment the composition according to the present invention comprises 1 μg p.p./ml peptide pool, 1 EU/ml LPS and 10 μg/ml poly(I:C). In a further preferred embodiment the composition according to the present invention comprises 1 μg p.p./ml peptide pool, 0.01 μg/ml IL-12, 1 EU/ml LPS and 10 μg/ml poly(I:C). In a further preferred embodiment the composition according to the present invention comprises 1 μg p.p./ml peptide pool, 1 EU/ml LPS, 10 μg/ml poly(I:C) and 100 mM urea. In a further preferred embodiment the composition according to the present invention comprises 1 μg p.p./ml peptide pool, 1 EU/ml LPS, 10 μg/ml poly(I:C), 0.01 μg/ml IL-12 and 100 mM urea. In a further preferred embodiment the composition according to the present invention comprises 1 μg p.p./ml peptide pool, 10 μg/ml poly(I:C), and 100 mM urea. In a further preferred embodiment the composition according to the present invention comprises 1 μg p.p./ml peptide pool, 0.01 μg/ml poly(I:C), and 0.01 μg/ml IL-12. In a further preferred embodiment the composition according to the present invention comprises 1 μg p.p./ml peptide pool, 1 EU/ml LPS, and 100 mM urea.

In a preferred embodiment the composition according to the present invention comprises 1 μg p.p./ml CEFTv pool, 1 EU/ml LPS and 10 μg/ml poly(I:C). In a further preferred embodiment the composition according to the present invention comprises 1 μg p.p./ml CEFTv pool, 0.01 μg/ml IL-12, 1 EU/ml LPS and 10 μg/ml poly(I:C). In a further preferred embodiment the composition according to the present invention comprises 1 μg p.p./ml CEFTv pool, 1 EU/ml LPS, 10 μg/ml poly(I:C) and 100 mM urea. In a further preferred embodiment the composition according to the present invention comprises 1 μg p.p./ml CEFTv pool, 1 EU/ml LPS, 10 μg/ml poly(I:C), 0.01 μg/ml IL-12 and 100 mM urea. In a further preferred embodiment the composition according to the present invention comprises 1 μg p.p./ml CEFTv pool, 10 μg/ml poly(I:C), and 100 mM urea. In a further preferred embodiment the composition according to the present invention comprises 1 μg p.p./ml CEFTv pool, 1 EU/ml LPS, and 100 mM urea. In a further preferred embodiment the composition according to the present invention comprises 1 μg p.p./ml CEFTv pool, 1 EU/ml LPS and 0.01 μg/ml IL-12. In a further preferred embodiment the composition according to the present invention comprises 1 μg p.p./ml CEFTv pool, 10 μg/ml poly(I:C) and 0.01 μg/ml IL-12.

In a preferred embodiment the composition according to the present invention comprises 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13, 14 or 15 μg/10⁸ cells of antibody or each antibody of a combination of two antibodies, preferably 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9 or 10 μg/10⁸ cells of anti-CD3 and/or 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13 14 or 15 μg/10⁸ cells of anti-CD28, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 EU/ml LPS and 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190 or 200 μg/ml of a poly(I:C) variant, preferably poly(I:C), poly(I:C)-LMW or poly(I:C)-LMW/LyoVec.

In a further preferred embodiment the composition according to the present invention comprises 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13, 14 or 15 μg/10⁸ cells of antibody or each antibody of a combination of two antibodies, preferably 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9 or 10 μg/10⁸ cells of anti-CD3 and/or 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13 14 or 15 μg/10⁸ cells of anti-CD28, 0.0001, 0.0005, 0.001, 0.005, 0.01, 0.05, 0.1, 0.2, 0.3, 0.4, 0.5, 1, 2, 3, 4 or 5 μg/ml IL-12, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 EU/ml LPS and 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190 or 200 μg/ml of a poly(I:C) variant, preferably poly(I:C), poly(I:C)-LMW or poly(I:C)-LMW/LyoVec.

In a further preferred embodiment the composition according to the present invention comprises 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13, 14 or 15 μg/10⁸ cells of antibody or each antibody of a combination of two antibodies, preferably 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9 or 10 μg/10⁸ cells of anti-CD3 and/or 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13 14 or 15 μg/10⁸ cells of anti-CD28, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 EU/ml LPS, 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190 or 200 μg/ml of a poly(I:C) variant, preferably poly(I:C), poly(I:C)-LMW or poly(I:C)-LMW/LyoVec and 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190, 200, 300, 400, 500, 600, 700, 800, 900 or 1000 mM urea.

In a further preferred embodiment the composition according to the present invention comprises 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13, 14 or 15 μg/10⁸ cells of antibody or each antibody of a combination of two antibodies, preferably 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9 or 10 μg/10⁸ cells of anti-CD3 and/or 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13 14 or 15 μg/10⁸ cells of anti-CD28, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 EU/ml LPS, 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190 or 200 μg/ml of a poly(I:C) variant, preferably poly(I:C), poly(I:C)-LMW or poly(I:C)-LMW/LyoVec, 0.0001, 0.0005, 0.001, 0.005, 0.01, 0.05, 0.1, 0.2, 0.3, 0.4, 0.5, 1, 2, 3, 4 or 5 μg/ml IL-12 and 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190, 200, 300, 400, 500 600, 700, 800, 900 or 1000 mM urea.

In a further preferred embodiment the composition according to the present invention comprises 00.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13, 14 or 15 μg/10⁸ cells of antibody or each antibody of a combination of two antibodies, preferably 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9 or 10 μg/10⁸ cells of anti-CD3 and/or 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13 14 or 15 μg/10⁸ cells of anti-CD28, 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190 or 200 μg/ml of a poly(I:C) variant, preferably poly(I:C), poly(I:C)-LMW or poly(I:C)-LMW/LyoVec, and 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190, 200, 300, 400, 500 600, 700, 800, 900 or 1000 mM urea.

In a further preferred embodiment the composition according to the present invention comprises 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13, 14 or 15 μg/10⁸ cells of antibody or each antibody of a combination of two antibodies, preferably 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9 or 10 μg/10⁸ cells of anti-CD3 and/or 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13 14 or 15 μg/10⁸ cells of anti-CD28, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 EU/ml LPS, and 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190, 200, 300, 400, 500, 600, 700, 800, 900 or 1000 mM urea.

In a further preferred embodiment the composition according to the present invention comprises 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13, 14 or 15 μg/10⁸ cells of antibody or each antibody of a combination of two antibodies, preferably 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9 or 10 μg/10⁸ cells of anti-CD3 and/or 0.05, 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13 14 or 15 μg/10⁸ cells of anti-CD28, 1, 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190 or 200 μg/ml of a poly(I:C) variant, preferably poly(I:C), poly(I:C)-LMW or poly(I:C)-LMW/LyoVec and 0.0001, 0.0005, 0.001, 0.005, 0.01, 0.05, 0.1, 0.2, 0.3, 0.4, 0.5, 1, 2, 3, 4 or 5 μg/ml IL-12.

In a preferred embodiment the composition according to the present invention comprises 30 ng/10⁸ cells of anti-CD3 or 0.76 μg/10⁸ cells of anti-CD3 and 3.8 μg/10⁸ cells of anti-CD28, 1 EU/ml LPS and 10 μg/ml poly(I:C). In a further preferred embodiment the composition according to the present invention comprises 30 ng/10⁸ cells of anti-CD3 or 0.76 μg/10⁸ cells of anti-CD3 and 3.8 μg/10⁸ cells of anti-CD28, 0.01 μg/ml IL-12, 1 EU/ml LPS and 10 μg/ml poly(I:C). In a further preferred embodiment the composition according to the present invention comprises 30 ng/10⁸ cells of anti-CD3 or 0.76 μg/10⁸ cells of anti-CD3 and 3.8 μg/10⁸ cells of anti-CD28, 1 EU/ml LPS, 10 μg/ml poly(I:C) and 100 mM urea. In a further preferred embodiment the composition according to the present invention comprises 30 ng/10⁸ cells of anti-CD3 or 0.76 μg/10⁸ cells of anti-CD3 and 3.8 μg/10⁸ cells of anti-CD28, 1 EU/ml LPS, 10 μg/ml poly(I:C), 0.01 μg/ml IL-12 and 100 mM urea. In a further preferred embodiment the composition according to the present invention comprises 30 ng/10⁸ cells of anti-CD3 or 0.76 μg/10⁸ cells of anti-CD3 and 3.8 μg/10⁸ cells of anti-CD28, 10 μg/ml poly(I:C), and 100 mM urea. In a further preferred embodiment the composition according to the present invention comprises 30 ng/10⁸ cells of anti-CD3 or 0.76 μg/10⁸ cells of anti-CD3 and 3.8 μg/10⁸ cells of anti-CD28, 1 EU/ml LPS, and 100 mM urea. In a further preferred embodiment the composition according to the present invention comprises 30 ng/10⁸ cells of anti-CD3 or 0.76 μg/10⁸ cells of anti-CD3 and 3.8 μg/10⁸ cells of anti-CD28, 1 EU/ml LPS and 0.01 μg/ml IL-12. In a further preferred embodiment the composition according to the present invention comprises 30 ng/10⁸ cells of anti-CD3 or 0.76 μg/10⁸ cells of anti-CD3 and 3.8 μg/10⁸ cells of anti-CD28, 10 μg/ml poly(I:C) and 0.01 μg/ml IL-12.

In still another preferred embodiment of the present invention, the composition further comprises a non-reducing sugar.

The present invention further relates to a method (i.e. an in vitro method) for measuring, determining and/or detecting the status of cell-mediated immune responsiveness of a subject, the method comprising

a) contacting a sample from the subject with the composition according to the present invention and

b) detecting the presence, elevation or decrease in the level of at least one immune effector molecule from immune cells, wherein the presence or level of the immune effector molecule is indicative of the level of cell-mediated responsiveness of the subject.

The immune effector molecule(s) according to the invention may be a nucleic acid or a protein or polypeptide, in particular an RNA, a DNA, a nucleic acid fragment and is induced by contact and incubation with the composition according to the invention. The immune effector molecule according to the present invention may be a cytokine, such as a lymphokine, interleukin or chemokine, such as such as IFN-β, INF-γ, TNF-α, IL-2, IL-4, IL-5, IL-6, IL-10, IL-12, IL-13, a colony stimulating factor (CSF) such as granulocyte (G)-CSF or granulocyte macrophage (GM)-CSF amongst many others such as complement or components in the complement pathway, TGF-β, MIP1a, MIP1b, 4-1BB, CD25, preferably soluble and/or membrane bound, perforin and/or granzyme. Preferably the immune effector molecule is interferon gamma (IFN-γ). Alternatively or additionally, the immune effector molecule may be a co-stimulatory molecule, such as a member of the TNF-receptor or TNF-ligand superfamily, such as 4-1BB Ligand (4-1BBL), preferably soluble or membrane-bound, OX40 ligand (OX40L), TNFSF (CD70), B7.1 (CD80), B7.2 (CD86), FcγRIII (CD16), FcγRII (CD32), FcγRI (CD64) or a further representative of the TNF/TNF-receptor and/or immunoglobulin superfamily or a member of the CXCL family e.g. CXCL9, CXCL10, CXCL11 or a member of the chemokine (C—C motif) ligand family e.g. CCL2, CCL 7, CCL8, CCL10 or IL1RN.

In a preferred embodiment the soluble immune effector molecules, preferably cytokines, more preferably IFN-β, INF-γ, TNF-α, IL-2, IL-4, IL-5, IL-6, IL-10, IL-12, IL-13, GM-CSF, TGF-β, MIP1a, MIP1b, 4-1BB, preferably soluble may be localized intracellular and/or extracellular.

The production of an immune effector molecule after stimulation with the composition can be determined by means of PCR, quantitative PCR (qPCR), reverse transcription quantitative real-time PCR (RT-qPCR), microarray, an ELISA, an ELISpot assay, multiplex bead assays and/or by means of FACS technology by determining for example intracellular cytokines, e.g. intracellular cytokine staining or secreted cytokines, e.g. FACS secretion assay.

The preferred method for detecting the presence of said immune effector molecule is the well-known ELISpot assay.

An immune effector molecule whose detection is particularly preferred in the context of the present invention is interferon gamma (IFN-γ) as this cytokine is produced by CD4+ T-cells, CD8+ T-cells and NK-cell. The detection method that is most preferred in the context of the present invention is ELISpot assay. ELISpot assays employ a technique very similar to the sandwich enzyme-linked immunosorbent assay (ELISA) technique. Either a monoclonal (preferred for greater specificity) or polyclonal capture antibody is coated onto a suitable membrane such as a PVDF (polyvinylidene fluoride) membrane. These antibodies are chosen for their specificity for the immune effector molecule in question. The membrane is blocked, usually with a serum protein that is non-reactive with any of the antibodies in the assay. After this, cells of interest are plated out at varying densities, along with a stimulating composition. Immune effector molecules secreted by activated cells is captured locally by the coated antibody on the membrane. After an optional washing steps to remove cells, debris, and media components, a second antibody specific for the immune effector molecule is added to the membrane. This antibody is typically reactive with a distinct epitope and is also modified by a detectable label, e.g. biotin, enzyme, tags, fluorescent markers. Following an optional washing step to remove unbound antibody, the detected immune effector molecule is visualized using the detectable label. The detectable end product typically represents an individual cytokine-producing cell. The spots can be counted manually, e.g., with a dissecting microscope or using an automated reader. Biotin is a preferred label. It is well known that biotin labelled antibodies need to be detected with a further entity which is able to specifically interact with biotin, e.g. streptavidin. Such systems are well known and include for example the streptavidin-ALP system. Thus, depending on the selected label it is envisaged to employ a further detection entity, e.g. detection of biotinylated antibodies via streptavidin.

In a preferred embodiment, the method of the present invention further comprises the following step c) comparing the detected immune effector molecule level with a reference-level.

The person skilled in the art is familiar with the concept of reference levels of immune effector molecules. In particular, the term reference level may relate to the actual value of the level in one or more control samples or it may relate to a value derived from the actual level in one or more control samples. Preferably, the number of samples is from at least 1, 2, 3, 4, 5, 6, 7, 8, 9 or 10, more preferably from at least 20, even more preferably from at least 50, most preferably from at least 100 subjects or more.

The control sample may be from one or more healthy subjects who does not undergo any of the treatments described herein or which does not suffer from any of the diseases as described herein, such as cancer or a bacterial, viral and/or fungal infection or which has not received a transplant.

However, the control sample may also be from a subject who undergoes any of the treatments described herein or who suffers from any of the diseases as described herein, such as cancer or a bacterial, viral and/or fungal infection, with the provision that the control sample is obtained from such a subject prior to any treatment or at the point in time when said subject is diagnosed to suffer from any of the diseases as described herein or prior to receipt of a transplant.

The reference level according to the method of the present invention may be the same as the level measured in the control sample or the average of the levels measured in a multitude of control samples. However, the reference level may also be calculated from more than one control sample. Preferably, the reference level relates to a range of levels that can be found in a plurality of comparable control samples, e.g. the average one or more times the standard deviation.

Similarly, the reference level may also be calculated by other statistical parameters or methods, for example as a defined percentile of the level found in a plurality of control samples, e.g. a 90%, 95%, 97.5%, or 99% percentile. The choice of a particular reference level may be determined according to the desired sensitivity, specificity or statistical significance. Calculation may be carried out according to statistical methods known and deemed appropriate by the person skilled in the art.

In a preferred embodiment of the method of the present invention, the sample is a body fluid, preferably from a mammalian subject. Accordingly, the body fluid is obtained from a mammalian subject. Examples of body fluids are lymph fluid, cerebral, fluid, tissue fluid, such as bone marrow or thymus fluid, respiratory fluid including nasal and pulmonary fluid and bronchoalveolar lavage, preferably the body fluid is whole blood or liquor. In another preferred embodiment the sample of the subject is anticoagulated blood, preferably whole blood or isolated peripheral mononuclear cells of the blood (PBMC). In another preferred embodiment the blood is anticoagulated by the addition of an anticoagulant, preferably novel oral anticoagulants (NOACs), e.g. dabigatran, rivaroxaban and apixaban; coumarins (vitamin K antagonists), as e.g. warfarin (Coumadin), acenocoumarol, phenprocoumon, atromentin and phenindione; heparin, synthetic pentasaccharide inhibitors of factor Xa, as e.g. fondaparinux and idraparinux; direct factor Xa inhibitors, as e.g. rivaroxaban, apixaban, edoxaban, darexaban and TAK-442 letaxaban and eribaxaban; direct thrombin inhibitors, as e.g. hirudin, lepirudin, bivalirudin, argatroban, dabigatran and ximelagatran; antithrombin protein therapeutics and other types of anticoagulants, as e.g. batroxobin and hementin. In an even more preferred embodiment the heparin is an ammonium, lithium or sodium heparin, most preferably a lithium heparin. More preferably, the sample of the subject is heparinized blood, preferably whole blood or isolated peripheral mononuclear cells of the blood (PBMC). In an even more preferred embodiment the sample of the subject is diluted or undiluted whole blood.

In a preferred embodiment, the sample comprises or consists of leucocytes, lymphocytes, or peripheral blood mononuclear cells (PBMCs). Preferably the sample comprises or consists of PBMCs.

The sample as described herein may comprise immune cells. Said immune cells are capable of producing at least one immune effector molecule. In a preferred embodiment of the method of the present invention, the immune cells comprised in the sample comprise at least T cells and NK-cells. T cells comprise at least CD8-positive T cells or CD4-positive T cells or both CD8-positive T cells and CD4-positive T cells.

Said immune cells may further comprise natural killer T-cells (NKT-cells). It is also contemplated that said immune cells are accompanied by antigen presenting cells (APCs). APCs support the immune cells. The sample may further comprise NKT-cells and/or APCs.

The subject according to the present invention may be a human or animal, preferably a mammal.

The subject according to the present invention may be immunosuppressed or immunodeficient. The subject according to the present invention may be immunosuppressed due to a natural immunosuppression or may be immunosuppressed due to therapeutic measures.

The subject according to the present invention may suffer from (i) cancer, or (ii) from a bacterial, viral and/or fungal infection, (iii) rheumatic disease, including rheumatoid arthritis, giant cell arthritis, reactive arthritis, undifferentiated oligoarthritis, polymyalgia rheumatic, acute sarcoidosis, ANCA-associated vasculitis and polychondritis (iv) chronic inflammatory disease (e.g. Morbus Crohn, Colitis ulcerosa), (v) systemic inflammatory response syndrome (SIRS), (vi) compensatory anti-inflammatory response syndrome (CARS)—both after sepsis or during pregnancy (vii) may be pregnant, (viii) at increased age, or (ix) may have received a transplant and/or is receiving a transplant. The bacterial, viral or fungal infection may be immunsuppressive.

Non-limiting examples of bacterial infections are infection with mycobacteria (e.g. M. tuberculosis) or MRSA. Non-limiting examples of viral infections are infection with CMV, EBV, VZV, HCV, HSV, HBV, HIV, HTLV.

The subject according to the present invention may be a critically ill subject or is an intensive care patient.

A further aspect of the present invention relates to a method (i.e. an in vitro method) for detecting the presence, absence, level or stage of a disease or condition in a subject, the method comprising a) contacting a sample of the subject with the composition according to the present invention and b) determining the presence, elevation or decrease in the level of an immune effector molecule from T-cells wherein the presence or level of the immune effector molecule is indicative of the disease or condition.

Another aspect of the present invention is a method (i.e. an in vitro method) for detecting the presence, absence, level or stage of a disease or condition in a subject in the presence of a potential immune stimulant contaminant such as an endotoxin or any of the mentioned first or second substances according to the invention the method comprising a) contacting a sample of the subject with the composition according to the present invention and b) determining the presence, elevation or decrease in the level of an immune effector molecule from T-cells wherein the presence or level of the immune effector molecule is indicative of the disease or condition.

A further aspect of the present invention relates to a method (i.e. an in vitro method) for measuring, determining and/or detecting cell-mediated immune response activity in a subject, the method comprising a) contacting a sample of the subject with the composition according to the invention and b) determining the presence, elevation or decrease in the level of an immune effector molecule from immune cells wherein the presence or level of the immune effector molecule is indicative of the level of cell-mediated responsiveness of the subject, wherein the level of responsiveness is indicative of the presence or absence or level or stage of a disease or condition selected from the list comprising an infection by a pathogenic agent, an autoimmune disease, a cancer, an inflammatory condition and exposure to a toxic agent.

In a preferred embodiment the contacting of the sample and the composition according to the present invention may be from 1 to 50 hours, such as 5 to 40 hours or 8 to 24 hours or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 hours.

In another preferred embodiment the sample used in the method according to the present invention comprises 10³ to 10²⁰ cells, preferably 10³ to 10¹² cells, more preferably 10⁵ to 10⁸ cells.

In another aspect, the present invention relates to a kit comprising the composition according to the present invention. Preferably, the kit according to the present invention comprises further substances, such as buffers, salts, solutions. More preferably the kit according to the present invention comprise further buffers, antibodies and membranes to perform the detection of immune effector cells, preferably to perform ELISA, or ELISpot assay.

In a preferred embodiment the concentration of the single components of the composition, i.e. first substance, second substance, LPS and/or urea are provided in the kit in a 2, 3, 4, 5, 6, 7, 8, 9, 10, 20, 30, 40 or 50 times higher concentration, preferably 10 times higher concentration than applied in the stimulation preparation.

In another embodiment the single components of the composition are provided in the kit in separate vials or tubes. This means that the first substance, the second substance, LPS and/or urea are each provided in a separate vial or tube in the kit.

In a further aspect, the present invention relates to the use of the composition according to the present invention for the production of a diagnostic composition or a kit.

A further aspect of the present invention relates to the use of the composition according to the present invention for measuring, determining and/or detecting cell-mediated immunity (CMI), preferably CMI mediated by CD8-positive T cells, CD4-positive T cells and NK-cells, more preferably CMI mediated by CD8-positive T cells, CD4-positive T cells and NK-cells irrespective of the infection status of the subject, preferably independent of the CMV-, EBV-, VZV-, HCV-, HCMV-, Influenza- and Clostridium tetani serostatus of the subject.

Another aspect of the present invention relates to the use of the composition according to the present invention for monitoring in vitro cell-mediated immunity, preferably over a course of time.

Another aspect of the present invention relates to the use of the composition according to the present invention for determining in vitro the immune status of a subject.

Another aspect of the present invention relates to the use of the composition according to the present invention for determining in vitro the immune status in an immunosuppressed subject for determining the risk of the occurrence of complications, such as CMV disease, graft loss, and opportunistic infections due to immunosuppression.

A further aspect of the present invention relates to the use of the composition according to the present invention for screening including selecting and/or detecting an immunosuppressive drug or compounds influencing the CMI.

A further aspect of the present invention relates to the use of the composition according to the present invention for monitoring of the treatment success in any therapy, preferably in a treatment with a medicament.

A further aspect of the present invention relates to the use of the composition according to the present invention for determining including selecting a treatment regimen or dosage of an immunosuppressive drug. Preferably, the immunosuppressive drug may be preselected. The treatment regimen is preferably for an immunosuppressed subject.

A further aspect of the present invention relates to the use of the composition according to the present invention for detecting, diagnosing, monitoring in vitro an immunosuppression condition in a subject.

A further aspect of the present invention relates to the use of the composition according to the present invention for determining and/or monitoring in vitro the cell-mediated immunological response in cancer immunotherapy or in response to a vaccine or in an autoimmune disease.

“Substance”, as used herein, may refer to a single compound as well as to a group of compounds. For example, the first substance may be a single compound selected from the group consisting of a protein, a peptide, and an antibody. Alternatively, the first substance may be a peptide pool comprising two or more peptides, or may even be a combination of two or more members of the group consisting of a protein, a peptide, a peptide pool and an antibody.

It is to be understood that both the foregoing general description and the following detailed description are exemplary and explanatory only and are not restrictive of the invention, as claimed.

The following examples explain the present invention but are not considered to be limiting.

### EXAMPLE 1: PREPARATION OF THE MONONUCLEAR CELLS OF PERIPHERAL BLOOD (PMBC)

The used peripheral blood lymphocytes are derived from an established collective of healthy CMV and EBC serotyped donors.

The table 7 lists the serostatus for EBV and CMV of the donors whose blood was used in the examples:

| Donors | EBV serostatus | CMV serostatus |
|---|---|---|
| d022 | − | − |
| d034 | + | + |
| d042 | + | − |
| d067 | − | − |
| d098 | + | − |
| d172 | + | + |
| d204 | + | + |
| d219 | + | − |
| d233 | + | − |
| d237 | + | − |
| d241 | + | + |
| d242 | + | + |
| d248 | + | − |
| d253 | + | − |
| d254 | + | + |
| d258 | − | − |
| d268 | − | + |
| d270 | + | + |
| d274 | + | − |
| d279 | − | − |

The mononuclear cells of the peripheral blood (PBMC) were obtained under sterile conditions using density gradient centrifugation from lithium heparin whole blood. Therefore 15 ml Pancoll were given in a 50 ml centrifugation tube (alternatively 17 ml Pancoll (PAN-Biotech GmbH, Aidenbach) were centrifuged in a blood separation tube (PAA Laboratories GmbH, Cölbe)) and overfilled with lithium heparin whole blood diluted 1:2 with PBS. Subsequently an unbraked centrifugation at 880×g for 30 minutes at room temperature followed (alternatively unbraked centrifugation of the blood separation tube at 840×g for 20 minutes at room temperature). Thereby the blood is separated in erythrocytes, PBMC and plasma. After centrifugation the PBMC is pipetted and transferred via a Pasteur pipette in a new 50 ml centrifugation tube. Subsequently the PBMC suspension is filled up with PBS to 50 ml and is sedimented at 300×g for 10 minutes at room temperature. The supernatant was decanted and the remaining cell pellet was resuspended with a pipette in 1 ml PBS, the cell suspension is then again filled up to 50 ml PBS and sedimented at 300×g for 10 minutes at room temperature. Then the supernatant was decanted and the remaining cell pellet is resuspended in 1 ml AIM-V media (Life Technologies Corporation, Carlsbad, Calif., USA).

### EXAMPLE 2: LIVE/DEAD STAINING

Per preparation 1 ml cell suspension (corresponding to PBMC adjusted to 1×10⁶ lymphocytes) were given in a 5 ml round bottom tube. Upon addition of the stimulants the stimulation occurred, wherein per used donor an unstimulated negative control and a positive control (addition of 3 μg/ml SEB Staphylococcus Enterotoxin B from Staphylococcus aureus (Sigma-Aldrich Co. LLC., St. Luis, Mo., USA)) was applied. Upon addition of the stimulants the samples were mixed by vortexing and incubated at 37° C. and 5% CO₂ for 19 hours. After incubation the samples were washed with 3 ml PBS (centrifugation at 300×g for 10 minutes at room temperature). After washing the supernatant was decanted and the cells suspended in 750 μl PBS. Then the live/dead staining with 5.5 μl of the mixture composed of 0.5 μl Sytox® Red (Life Technologies Corporation, Carlsbad, Calif., USA) and 5 μl Annexin V FITC (Becton, Dickinson and Company, Franklin Lakes, N.J., USA) per preparation was performed. The samples were mixed by vortexing and incubated at 4° C. in dark for 15 minutes. Afterwards the samples were analyzed in the flow cytometer.

The test method was performed under sterile conditions.

### EXAMPLE 3: METHODS FOR DETECTION OF IFN-γ SECRETING CELLS

ELISpot Assay (Enzyme Linked Immunospot Assay)

96 well PVDF (polyvinylidenfluorid) micro titer plates (Millipore GmbH, Schwalbach) were wet out at the beginning for one minute with 15 μl 35% ethanol per well. Subsequently the plates were washed three times with 200 μl PBS per well and then 100 μl per well of the anti-human interferon-g mAb 1-D1K (Mabtech AB, Hamburg) in an end concentration of 5 μg/ml (diluted in PBS) was added. The plates were then closed and stored at 4° C. over night.

On the next day the antibodies were removed by pipetting and the plates were washed twice with 150 μl USP WFI (water for cell culture) (Lonza Group Ltd., Basel, Schweiz) per well. Subsequently 200 μl AIM-V medium per well was added and the plates were incubated for 2 hours at 37° C. and 5% CO₂ to block unspecific binding sites. After blocking 50 μl stimulant in the desired concentration (diluted in AIM-V from Life Technologies Corporation (Carlsbad, Calif., USA)) was added per preparation. As negative control cells incubated with AIM-V medium was used. After addition of the stimulants 100 μl of the cell suspension per well in a concentration of PBMC adjusted to 2×10⁵ lymphocytes were added. Last, the positive control was added in form of 2 μg/ml SEB (Staphylococcus Enterotoxin B from Staphylococcus aureus (Sigma-Aldrich Co. LLC., St. Luis, Mo., USA) (diluted in AIM-V) and the plates were incubated for 19 hours at 37° C. and 5% CO₂. After incubation the cell suspension was removed and the plates were washed six times for each 3 minutes with 200 μl 0.01% Tween20 (Merck KGaA, Darmstadt) per well. Then the detection of the IFN-γ secreting cells was performed, wherein it is distinguished between a one-step and two-step detection method. The one-step detection method exhibits a lower sensitivity for detection of IFN-γ producing cells in a clearly reduced unspecific background.

One-Step Detection:

100 μl detection conjugate MAb<h-IFN-γ>M-7-B6-1-IgG-AP (MicroCoat GmbH, Bernried) were added in a concentration of 0.4 U/ml (diluted in detection conjugate buffer 1 (MicroCoat GmbH, Bernried) per well and incubated for two hours at 37° C. and 5% CO₂. Then it was washed three times for each 3 minutes with 200 μl 0.01 Tween20 (Merck KGaA, Darmstadt) (diluted in PBSo) per well and then washed three times each 3 minutes with 200 μl PBSo (137 mmol/1 NaCl, 2.7 mmol/1 KCl, 8.0 mmol/1 Na₂HPO₄×2H₂O, 1.47 mmol/1 KH₂PO₄, pH 7.4).

Two-Step Detection:

100 μl biotinylated antibody anti-human IFN-γ mAb 7-B6-1 Biotin (Mabtech AB, Hamburg) was added per well in a concentration of 1 μg/ml (diluted in detection conjugate buffer 2 (MicroCoat GmbH, Bernried) and was incubated for two hours at room temperature. Then it was washed three times for each 3 minutes with 200 μl 0.01 Tween20 (Merck KGaA, Darmstadt) (diluted in PBSo) per well and then washed three times each 3 minutes with 200 μl PBSo (137 mmol/1 NaCl, 2.7 mmol/1 KCl, 8.0 mmol/1 Na₂HPO₄×2H₂O, 1.47 mmol/1 KH₂PO₄, pH 7.4). Then 100 μl streptavidin-ALP (Mabtech AB, Hamburg) in a concentration of 1 μg/ml (diluted in detection conjugate buffer 2) was added per well. After one hour incubation at room temperature the wells were washed six times each for 3 minutes with 200 μl PBSo (137 mmol/1 NaCl, 2.7 mmol/1 KCl, 8.0 mmol/1 Na₂HPO₄×2H₂O, 1.47 mmol/1 KH₂PO₄, pH 7.4) per well.

Finally (both after the one-step and two-step detection), the staining reaction followed with 50 μl 1-Step NBT/BCIP (Thermo Fisher Scientific Inc., Waltham, Mass., USA) at which the wells are incubated for six minutes at room temperature in the dark. The staining reaction was stopped by rinsing the plates with water three times. The plates were then beaten on a tissue, the bottom plate was removed and the membrane was carefully pressed on the tissue.

The analysis of the plates was performed using the BioReader 5000-Ea (Bio-Sys GmbH Karben) after drying of the plates over night.

The experiment was performed under sterile conditions.

ELISA (Enzyme Linked Immunosorbent Assay)

The detection of secreted IFN-γ was performed with the test kit “BD OptEIA Human IFN-γ ELISA Set” from Becton, Dickinson and Company according to the recommended assay performance in the technical data sheet.

Intracellular Cytokine Staining

Per preparation 1 ml cell suspension (corresponding to PBMC adjusted to 1×10⁶ lymphocytes) were given in a 5 ml round bottom tube. Per donor a preparation with 3 μg/ml SEB (Staphylococcus Enterotoxin B from Staphylococcus aureus (Sigma-Aldrich Co. LLC., St. Luis, Mo., USA) served as positive control and an unstimulated preparation as negative control. The remaining preparations were incubated dependent on the aim with one or more specific stimulants. After addition of the stimulants the samples were mixed by vortexing. Each stimulation was done twice and was incubated for 8 and 18 hours respectively at 37° C. and 5% CO₂. Six hours prior to the end of the incubation time 20 μl BFA (Sigma-Aldrich Co. LLC., St. Luis, Mo., USA) in a concentration of 0.5 μg/μ1 (diluted in DMSO, diluted in PBS) was added per preparation, the samples were mixed by vortexing and then incubated further at 37° C. and 5% CO₂. After incubation the sample were washed each with 3 ml PBS (centrifugation at 350×g for 6 minutes at 4° C.; after incubation the supernatant was decanted and the pellet was suspended by mixing using vortex). After washing the surface staining was performed with each 40 μl of a mixture of 5 μl CD3 APC-AF750 (Human CD3 APC-Alexa Fluor® 750 Conj., Life Technologies Corporation, Carlsbad, Calif., USA), 10 μl CD4 ECD (Beckman Coulter Inc., Krefeld), 5 μl CD8 APC (eBiosciences GmbH, Frankfurt) and 20 μl CD56 PE (Becton, Dickinson and Company, Franklin Lakes, N.J., USA). After mixing of the samples they were incubated at room temperature in the dark for 20 minutes. After incubation the cells were washed again with each 3 ml PBS. Afterwards the cells were fixed with each 1 ml 1× Fix/Perm Buffer (diluted in TF Diluent Buffer) and permeabilized, mixed by vortexing for 3 seconds and incubated in the dark at 4° C. for 45 minutes. After incubation the cells were washed first with each 1 ml 1× Perm/Wash Buffer (diluted in TF Diluent Buffer) and then with 2 ml 1× Per/Wash Buffer. The intracellular staining was performed with each 81 μl of a mixture of 80 μl 1. Perm/Wash Buffer and 1 μl IFN-γ FITC (Klon B27) (Becton, Dickinson and Company, Franklin Lakes, N.J., USA). After mixing for 10 seconds by vortexing these samples were incubated in the dark at 4° C. for 45 minutes. Two washing steps followed with each 2 ml 1× Perm/Wash Buffer. The cells were finally suspended in each 750 μl 1% PFA (diluted in PBSo (137 mmol/1 NaCl, 2.7 mmol/1 KCl, 8.0 mmol/1 Na₂HPO₄×2H₂O, 1.47 mmol/1 KH₂PO₄, pH 7.4)) and stored at 4° C. in the dark over night until analysis in the flow cytometer.

### EXAMPLE 4: STIMULATING ACTIVITY OF DISTINCT POLY(I:C) VARIANTS

In this experiment the immune activating properties of distinct poly(I:C) variants inducing the IFN-γ production of NK cells were analyzed. The following three distinct poly(I:C) variants were used: poly(I:C)LWM (from InvivoGen San Diego, Calif., USA), poly(I:C) (from Enzo Life Sciences GmbH, Lörrach) and poly(I:C)-LMW/LyoVec (from InvivoGen San Diego, Calif., USA).

Poly(I:C)LMW (from Invivogen) activates cells additionally via binding to the receptor TLR3, poly(I:C) (from Enzo) activates cells additionally via binding to the receptor MDA5 and poly(I:C)-LMW/LyoVec (from Invivogen) is a transfected poly(I:C) which mainly binds to the receptors RIG-I and MDA-5 and thus activates the cells.

The PBMC used in this experiment were obtained and cultured as described in example 1.

To compare the stimulation properties of the three poly(I:C) variants and simultaneously determine the optimal working concentration, each variant was titrated in half logarithmic steps starting at a concentration of 3.16×10⁻⁴ μg/ml to a concentration of maximum 10 and 31.6 μg/ml respectively (dependent on the poly(I:C) variant). Each concentration of the three poly(I:C) variants was subsequently incubated with PBMC adjusted to 2×10⁵ lymphocytes for 19 hours at 37° C. and 5% CO₂. The detection of IFN-γ secreting cells was performed using the ELISpot assay both in a one-step and two-step manner as described in Example 3. Each poly(I:C) variant was tested with PBMC of 3 donors (d042, d098 and d233).

Using the one-step detection method an activation of IFN-γ secreting PBMC from donor d042 was observed after stimulation with poly(I:C)-LMW (from Invivogen) and poly(I:C) (from Enzo). In case of stimulation with poly(I:C) (from Enzo) a maximum of IFN-γ secreting cells around 0.3 μg/ml could be observed. In case of stimulation PBMC of donor d098 with poly(I:C)-LMW (from Invivogen) it could be observed that the number of IFN-γ secreting cells reached a plateau as from 3 μg/ml. The number of IFN-γ secreting cells stimulated by poly(I:C) (from Enzo) exceeded this plateau at the highest concentration tested of 10 μg/ml by more than 100%. Upon stimulation with poly(I:C)-LMW/LyoVec the PBMS of donor d098 did not react. The PBMC of donor d233 reacted on none of the used poly(I:C) variants with a measurable activation of IFN-γ secreting cells.

Using the two-step detection method more IFN-γ secreting cells were detected after stimulation of the PBMC of all donors. Upon stimulation of the PBMC of donor d042 with poly(I:C) (Enzo) the maximum of IFN-γ secreting cells was reached at concentrations of 0.3 to 1 μg/ml. Upon stimulation with poly(I:C)-LMW (from Invivogen) the number of IFN-γ secreting cells increased to the highest concentration tested. Upon stimulation of the PBMC of donor d098 with poly(I:C)-LMW (from Invivogen) the number of IFN-γ secreting cells reached a plateau at concentration of 10 μg/ml. In PBMC stimulated with poly(I:C) (from Enzo) higher number of IFN-γ secreting cells could be observed, which increased with increasing poly(I:C) concentrations without reaching a plateau. The PBMC of donor d233 did not reacted even using higher doses of poly(I:C) and the more sensitive detection system with a measurable activation of IFN-γ producing cells. The strongest stimulation of the PBMC of donor d042 was observed with 1 μg/ml poly(I:C)-LMW/LyoVec (from Invivogen).

In summary it could be shown that the use of poly(I:C) (from Enzo) resulted in the highest activation of IFN-γ secreting cells in culture of human PBMC followed by poly(I:C)-LMW (from Invivogen). Upon stimulation with poly(I:C)-LMW/LyoVec no IFN-γ secreting cells could be detected using the one-step detection method. But using the two-step detection method a number of IFN-γ secreting cells similar high as measured after stimulation with poly(I:C)-LMW (from Invivogen). The following working concentrations lying in a stable saturation region have been selected for further experiments:

| | |
|---|---|
| Poly(I:C)-LMW/LyoVec (from Invivogen) | 1 μg/ml |
| Poly(I:C)-LMW (from Invivogen) | 10 μg/ml |
| Poly(I:C) (Enzo) | 1 μg/ml |

### EXAMPLE 5: INFLUENCE OF IL-12 ON THE STIMULATING PROPERTIES OF POLY(I:C)

It is already described in the literature that the production of IFN-γ in NK cells may be enhanced upon stimulation with poly(I:C) if IL-12 is added (Girart et al., 2007).

The PBMC used in this experiment were obtained and cultured as described in example 1.

For analysis of the influence of IL-12 on IFN-γ secreting cells induced by poly(I:C) in PBMC, PBMC of three donors adjusted to 2×10⁵ lymphocytes were incubated with half-logarithmic increasing concentrations of IL-12 in presence and absence of various poly(I:C) variants for 19 hours at 37° C. and 5% CO₂. The detection of IFN-γ secreting cells was performed using the ELISpot assay both in a one-step and two-step manner as described in example 3.

Using the one-step detection method it could be observed in PBMC of donor 098 that IL-12 alone could not stimulate a significant number of cell to produce a measurable IFN-γ secretion. In combination with 10 μg/ml poly(I:C)-LMW (from InvivoGen San Diego, Calif., USA) the number of IFN-γ secreting cells was increased significantly and reached a maximum of a IL-12 concentration of 0.03 μg/ml. In combination with 1 μg/ml poly(I:C)-LMW/LyoVec (from InvivoGen San Diego, Calif., USA) and with 1 μg/ml poly(I:C) (from Enzo Life Sciences GmbH, Lörrach), respectively a very low increase of the number of IFN-γ secreting cells was observed in comparison with 10 μg/ml poly(I:C)-LMW (from Invivogen).

Using the two-step detection method no activation of the IFN-γ secreting cells in all tested donors upon stimulation of the PBMC with IL-12 was observed. The combination of 10 μg/ml poly(I:C)-LMW (from Invivogen) with IL-12 induced the highest numbers of IFN-γ secreting cells in PBMC. Thereby the IFN-γ secreting cells of donor d022 and d248 reached at a concentration of 0.003 μg/ml IL-12 a plateau, in donor d098 the maximum of stimulated cells has been reached at a concentration of 0.01 μg/ml IL-12. IL-12 in combination with 1 μg/ml poly(I:C)-LMW/LyoVec (from Invivogen) and with 1 μg/ml poly(I:C) (from Enzo) respectively induced upon stimulation of the PBMC of donor d022 almost the identical number of IFN-γ secreting cells by reaching a plateau from a concentration of 0.01 μg/ml. The PBMC of donor d098 were stimulated similarly by these combinations, wherein by 1 μg/ml poly(I:C) (from Enzo) the number of induced IFN-γ secreting cells reached a plateau already at a IL-12 concentration of 0.00 μg/ml. With 1 μg/ml poly(I:C)-LMW/LyoVec (from Invivogen) the stimulated cells reached a plateau at a IL-12 concentration of 0.003 μg/ml, although this was higher compared to 1 μg/ml poly(I:C) (from Enzo) with IL-12. The stimulation of the PBMC of donor d248 showed that again a similar course of the IFN-γ secreting cells was observed, which reached a plateau at a IL-12 concentration of 0.01 μg/ml with the distinction that at 1 μg/ml poly(I:C)-LMW/LyoVec (from Invivogen) the number of stimulated cells was 100% higher.

This experiment showed that advantageous stimulation results are obtained by combination of 10 μg/ml poly(I:C)-LMW (from Invivogen) with IL-12.

### EXAMPLE 6: STIMULATING ACTIVITY OF DISTINCT A-GAL-CER VARIANTS

The a-Gal-Cer (alpha Galactosylceramide) is a specific ligand which is presented by the MHC-like molecule CD1d. This complex is recognized by a specific T cell receptor type of human and murine NKT cells (natural killer T cells) and thus leading to a production of Th1 and Th2 cytokines. For this experiment, the synthetic a-Gal-Cer analogue KRN7000 from two different manufacturer was used to analyze differences due to production or manufacturer. The two tested variants are: KRN7000 from Enzo Life Sciences Inc. and KRN70000 from Funakoshi Co., Ltd. Additionally the a-Gal-Cer analogue 8 from Enzo Life Sciences Inc. was tested, which induced lower amounts of IFN-γ in mouse models according to the manufacturer, but same or higher amounts of IL-4 in comparison to the KRN7000.

The PBMC used in this experiment were obtained and cultured as described in example 1.

To compare the stimulation properties of the a-Gal-Cer variants and simultaneously determine the optimal working concentration, each variant was titrated in half logarithmic steps starting at a concentration of 0.01 μg/ml to a concentration of 31.6 μg/ml (dependent on the a-Gal-Cer variant). Each concentration of the three a-Gal-Cer variants was subsequently incubated with PBMC adjusted to 2×10⁵ lymphocytes for 19 hours at 37° C. and 5% CO₂. The detection of IFN-γ secreting cells was performed using the ELISpot assay both in a one-step and two-step manner as described in example 3. Each a-Gal-Cer variant was tested with PBMC of 3 donors (d022, d242 and d248).

Using the one-step detection method the PBMC of donor d022 showed with all three a-Gal-Cer variants a similar stimulation course of the IFN-γ secreting cells, wherein the saturation region was shifted each by a half log step. The cells stimulated by KRN7000 (from Funakoshi) reached the saturation region already at a concentration of 1 μg/ml followed by the cells stimulated by KRN7000 (from Enzo) at a concentration of 3 μg/ml. The cells stimulated by a-Gal-Cer analogue 8 (from Enzo) did not reach a clear saturation region. The number of IFN-γ secreting cells in the saturation region was similarly high upon stimulation with both KRN7000 variants, wherein the number of IFN-γ secreting cells induced by a-Gal-Cer analogue 8 was a bit lower.

The PBMC of the donor d242 did not react on any of the used a-Gal-Cer variants with a IFN-γ production. Upon stimulation of the PBMC of donor d248 with both KRN7000 variants the number IFN-γ secreting cells reached the saturation region at a concentration of 3 μg/ml, wherein 100% more IFN-γ secreting cells has been activated by KRN7000 (from Funakoshi) compared with KRN7000 (from Enzo). The cells stimulated by a-Gal-Cer analogue 8 did not reach a saturation region. The decrease of the number of stimulated cells at a concentration of 30 μg/ml was caused by a too high concentration of DMSO (about 3% v/v) and the resulting cytotoxic effect.

Using the two-step detection method a similar course of the stimulated cells could be observed upon stimulation of the PBMC of donor d022 in comparison to the one-step detection method. The cells stimulated by both KRN7000 variants reached both at a concentration of 3 μg/ml a plateau this time. The cells stimulated by a-Gal-Cer analogue 8 again reached a clear recognizable saturation region. KRN7000 (from Enzo) induced the highest number of IFN-γ secreting cells followed by KRN7000 (from Funakoshi). The lowest number of IFN-γ secreting cells has been activated by a-Gal-Cer analogue 8 (from Enzo). Upon stimulation of the PBMC of donor d242 an increase of IFN-γ secreting in the highest non-toxic concentration could be observed by all three a-Gal-Cer variants. The highest number of IFN-γ secreting cells was induced by KRN7000 (from Funakoshi) followed by KRN7000 (from Enzo). The lowest rate of stimulated cells was activated by the a-Gal-Cer analogue 8. The PBMC of donor d248 has been activated similarly by the three a-Gal-Cer variants. No saturation region was reached upon titration of the three a-Gal-Cer variants. The course of the IFN-γ secreting cells induced by both KRN7000 variants was identical. The course of the IFN-γ secreting cells induced by the a-Gal-Cer analogue 8 (from Enzo) was shifted by one log step. The decrease of the number of stimulated cells at a concentration of 30 μg/ml was caused by a too high concentration of DMSO (about 3% v/v) and the resulting cytotoxic effect.

In summary, this experiment showed that both KRN7000 variants stimulated a similarly high number of IFN-γ secreting cells in PBMC. Only in the one-step detection method more IFN-γ secreting cells has been detected upon stimulation of the PBMC of donor d248 with KRN7000 (from Funakoshi) than upon stimulation with KRN7000 (from Enzo). The a-Gal-Cer analogue 8 (from Enzo) induced the least IFN-γ secreting cells in PBMC of all donors.

The working concentration of KRN7000 (from Funakoshi) was set to 10 μg/ml for the following experiments and thus lying in a stable saturation region.

### EXAMPLE 7: STIMULATING ACTIVITY OF DISTINCT PEPTIDE MIXTURES

For the stimulation of T helper and cytotoxic T cells peptide mixtures has been tested. The peptides present in the peptide mixtures each represent a pathogen specific epitope and are presented on T cells using MHC complexes.

The PBMC used in this experiment were obtained and cultured as described in example 1.

Three distinct peptide mixtures were tested, which are distinct in their composition and length of the peptides. The peptide mixture CEF is composed of 23 peptides whose peptide sequences are derived from various proteins of the cytomegalic virus (CMV), the Epstein Barr virus (EBV) and the influenza virus. The peptide mixture CEFT comprised besides the 23 peptides used in CEF 4 additional peptides, whose peptide sequences are derived from the bacteria Clostridium tetani. The peptide mixture CEFTv was composed of the same peptides like the peptide mixture CEFT except that the naturally occurring peptide sequences are elongated by 5 amino acids on the N-terminal and C-terminal end of the peptides. It was the intention that the elongated peptides would not fit in the binding pocket of the MHC molecules and thus they have to be degraded by the antigen presenting cells (APC) prior to the presentation via MHC molecules. Thereby a presentation of epitope variants is expected which would lead to a broader activation of IFN-γ secreting cells.

To compare the stimulation properties of the three peptide mixtures and simultaneously determine the optimal working concentration, each mixture was titrated in half logarithmic steps starting at a concentration of 1×10⁻⁵ μg p.p./ml (μg per peptide/ml) to a concentration of 100 μg p.p./ml (dependent on the peptide mixture). Additionally preparations were performed with a concentration of 0.316 and 3.16 μg p.p./ml to illustrate the course of IFN-γ secreting cells in this region in more detail. Each concentration of the three peptide mixtures was subsequently incubated with PBMC adjusted to 2×10⁵ lymphocytes for 19 hours at 37° C. and 5% CO₂. The detection of IFN-γ secreting cells was performed using the ELISpot assay both in a one-step and two-step manner as described in example 3. Each peptide mixture was tested with PBMC of 4 donors (d022, d204, d219 and d254).

Using the one-step detection method, almost no IFN-γ secreting cells have been detected upon stimulation of PMBC of donor d022 with the peptide mixtures. The stimulation with CEFTv resulted in a low maximum of IFN-γ secreting cells at 1 μg p.p./ml. The PBMC of donor d204 were almost identically stimulated by CEFT and CEFTv and the IFN-γ secreting cells reached a plateau as from concentration of 0.01 μg p.p./ml. Upon stimulation of the PBMC with CEF the number of IFN-γ secreting cells did not reach a saturation region until the highest tested non-toxic concentration and exceeded in this concentration the highest number of IFN-γ secreting cells stimulated by CEFT and CEFTv. The PBMC of donor d219 did not react with a measurable activation of IFN-γ secreting cells due to the peptide mixtures. The only exception formed the stimulation of the PBMC with CEFT in a concentration of 10 μg p.p./ml at which a maximum of IFN-γ secreting cells was reached. The PBMC of donor d254 reacted only on CEFTv and the number of stimulated cells reached a saturation region as from 0.1 μg p.p./ml. The decrease of the number of stimulated cells at a concentration of 100 μg p.p./ml was caused by a too high concentration of DMSO (about 14% v/v) and the resulting cytotoxic effect.

Using the two-step detection method, the number of IFN-γ secreting cells was again low upon stimulation of the PBMC of donor d022. Upon stimulation of the PBMC with CEFTv a maximum of IFN-γ secreting cells was detected at a concentration of 0.3 μg p.p./ml upon a stimulation with CEFT a maximum was detected at a concentration of 10 μg p.p./ml. The PBMC of donor d204 showed a comparable IFN-γ secretion on both peptide mixtures. The plateau of IFN-γ secreting cells started at a concentration of 0.3 μg p.p./ml, wherein the cells stimulated by CEFT formed a slightly higher plateau. The PBMC of donor d219 again reacted very weakly on both peptide mixtures. The cells stimulated by CEFTv reached a plateau as from a concentration of 0.3 μg p.p./ml. The cells stimulated by CEFT reached a maximal value at 3 μg p.p./ml which exceeded the plateau of the cells stimulated by CEFT more than nearly 100%. The stimulation of the PBMC of donor d254 with CEFT induced a low number of IFN-γ secreting cells which achieved its maximal value at the highest non-toxic concentration of 10 μg p.p./ml. The PBMC were stimulated best with CEFTv. The stimulated cells again reached a plateau as from a concentration of 0.1 μg p.p./ml, which exceeded the highest number of IFN-γ secreting cells induced by CEFT by 100%. Also using the two-step detection method, the decrease of the number of stimulated cells at a concentration of 100 μg p.p./ml was caused by a too high concentration of DMSO and the resulting cytotoxic effect.

In summary the experiments showed that the highest number of IFN-γ secreting cells were detected upon stimulation of the PBMC of CMV seropositive donors. On the contrary, the PBMC of CMV seronegative donors d022 and d204 were stimulated weakly or not at all. A special case was represented by CMV-/EBV-seropositive donor d254 whose PBMC only reacted on the peptide mixture CEFTv. The working concentration for CEFTv for the following experiments was determined to be 0.1 μg p.p./ml.

### EXAMPLE 8: DETERMINATION OF THE TEMPORAL COURSE OF IFN-γ RELEASE INDUCED BY THE SPECIFIC STIMULANTS

To ensure that the activation of the IFN-γ production by the tested stimulants occurred in the typical stimulation period of 19 hours, the temporal course of the IFN-γ secretion induced by the stimulants was determined over a period of 24 hours. For this purpose, at each time point PBMC adjusted to 2×10⁵ lymphocytes of different donors have been stimulated by the determined working concentrations of the stimulants poly(I:C) in combination with IL-12, KRN7000 and CEFTv for 0 to 24 hours.

The PBMC used in this experiment were obtained and cultured as described in example 1.

In specifically stimulated cells the determination of the IFN-γ production was made to 11 time points in an interval of 2 or 4 hours. In unstimulated cells (negative control) three measurements in interval of 6 and 12 hours were made. Upon completion of each stimulation period the cells of the stimulation preparation were centrifuged and the supernatant stored at −20° C. After all stimulations were finished the IFN-γ concentration of the supernatants has been determined by IFN-γ ELISA as described in example 3.

Upon stimulation of the PBMC with poly(I:C) in combination with IL-12 the IFN-γ secretion of the PBMC of donor d022 started between 8 and 12 hours and increased over the remaining time. The PBMC of donor d098 started the IFN-γ secretion also between 8 and 12 hours, and increased markedly as from 18 hours. The PBMC of donor d248 showed in comparison a very low IFN-γ secretion which started after 12 hours and increased slightly over the remaining time.

Upon stimulation of the PBMC with KRN7000 the IFN-γ secretion of the PBMC of donor d022 started after 8 hours and reached a plateau after 12 hours which decreased after 16 hours. The IFN-γ secretion of the PBMC of donor d242 reached a maximum between 18 and 22 hours which was very low compared with the negative controls. These data points were characterized by a high variation. The IFN-γ secretion of the PBMC of donor d248 showed a slight increase after 2 hours, which reached a maximum after 18 hours which is characterized by a high variation. Afterwards a decrease could be observed.

Upon stimulation of the PBMC of CMV-seronegative donors with CEFTv a release of IFN-γ was hardly detected. Only the PBMC of donor d022 secreted low amounts of IFN-γ after 6 hours, but the time point was characterized by a high variation. The PBMC of donor d219 only exhibited a very low IFN-γ secretion in the time frame of 12 and 22 hours. Contrarily, the PBMC of both CMV-seropositive donors showed a markedly IFN-γ release upon stimulation. The PBMC of both donors secreted the first time significant amounts of IFN-γ after 8 hours. The concentration of IFN-γ secreted by PBMC of donor d204 reached a plateau at 0.03 ng/ml after 16 hours. The IFN-γ secreted by PBMC of donor d254 reached a lower plateau at 0.02 ng/ml after 12 hours.

In summary, it became clear that poly(I:C) in combination with IL-12 in comparison with KRN7000 and CEFTv induced the strongest IFN-γ release in PBMC. The IFN-γ secretion was detectable between 6 and 8 hours and increased over the remaining time. KRN7000 showed the weakest IFN-γ activating properties, wherein the maximum was reached between 12 and 22 hours dependent from the donor. Upon stimulation of the PBMC with CEFTv a significant higher IFN-γ secretion in PBMC of CMV-seropositive donors was measured, wherein the highest concentrations of IFN-γ were detected after 12 to 16 hours. The working concentration of CEFTv was set to 1 μg p.p./ml for the following experiments, since the IFN-γ production induced by CEFTv was low.

### EXAMPLE 9: ANALYSIS OF THE SELECTED STIMULANTS FOR CELL-DAMAGING EFFECTS

To exclude that the stimulants may have cell-damaging effects on lymphocytes a live/dead staining was performed with the selected working concentrations of the stimulants as described in detail in the examples 4 to 8. The live/dead staining is composed of the staining of DNA using Sytox Red, as well as the staining of phophatidylserine with Annexin V-FITC.

The PBMC used in this experiment were obtained and cultured as described in example 1.

The live/dead staining was performed after the PBMC of the donor has been incubated with the cell-specific stimulants for 19 hours as described in example 2. The stimulation period of 19 hours should thereby simulate the incubation period of the ELISpot assay as described in example 3.

Due to the comparison with the unstimulated control it could be observed that none of the tested stimulants has significant cytotoxic properties on lymphocytes. The population of apoptotic cells could contain also vital cytotoxic T cells which turned phosphatidylserine outside due to a T cell receptor mediated antigen recognition (Fischer et al., 2006).

### EXAMPLE 10: ANALYSIS OF THE INFLUENCE OF UREA BUFFER ON THE STIMULATING PROPERTIES OF CEFTV AT DIFFERENT PRE-INCUBATION CONDITIONS

In this experiment, it was analyzed if the addition of urea may improve the ability of peptides to specifically activate T cells.

The PBMC used in this experiment were obtained and cultured as described in example 1.

For analysis of the influence of urea on the activation of IFN-γ secreting cells induced by CEFTv, first 1 μg p.p./ml CEFTv in different strongly concentrated urea buffer was incubated for up to 48 hours at 37° C. and 5% CO₂. After the incubation the PBMC adjusted to 2×10⁵ lymphocytes of 4 donors were added and further incubated for 19 hours at 37° C. and 5% CO₂. The number of IFN-γ secreting cells has been detected with the ELISpot assay in a two-step manner as described in example 3.

In the donors d022 and d233 the number of IFN-γ secreting cells was increased due to the stimulation of the PBMC with CEFTv and urea with increasing concentration in all tested pre-incubation conditions (without, 24 and 48 hours pre-incubation). In donor d034 the activation of IFN-γ secreting cells in PBMC was only improved by a 24 hours pre-incubation of CEFTv or directly added urea in the stimulation preparation. The stimulation of the PBMC with CEFTv pre-incubated with urea buffer for 48 hours showed no increase of the number of IFN-γ secreting cells compared with a urea-free stimulation preparation with CEFTv. But it could be observed that the stimulating properties of CEFTv was improved by pre-incubation at 37° C. and 5% CO₂ with increasing pre-incubation time. This effect was not observed in the PBMC of any of the other donors. In donor d204 the addition of urea without pre-incubation resulted in no improvement of the cell-stimulating properties of CEFTv. However, if the PBMC of donor d204 was stimulated with CEFTv pre-incubated in urea buffer for 24 hours an increasing course of the IFN-γ secreting cells up to a concentration of 12.5 mM could be observed which was parabolic as from this concentration. Upon stimulation with CEFTv which was pre-incubated for 48 hours in urea buffer no stimulating influences were detected except a deterioration of the stimulation at 100 mM and an improvement at 150 mM urea.

The experiment showed that a pre-incubation of CEFTv in urea buffer at 37° C. and 5% CO₂ only resulted in more IFN-γ secreting cells in PBMC of donor d034 upon stimulation. Upon stimulation of the PBMC of the other three donors CEFTv induced lower numbers of IFN-γ secreting cells the longer CEFTv was pre-incubated in urea.

The working concentration for urea was set to 100 mM without pre-incubation for the following experiments.

### EXAMPLE 11: ANALYSIS OF THE INFLUENCE OF LPS ON THE STIMULATING PROPERTIES OF CEFTV

In this experiment, it should be examined if the IFN-γ secreting cells induced by CEFTv could be increased by the addition of LPS (lipopolysaccharide).

The PBMC used in this experiment were obtained and cultured as described in example 1.

For analysis of the influence of LPS on the activation of IFN-γ secreting cells induced by CEFTv, the PBMC adjusted to 2×10⁵ lymphocytes of 4 donors were incubated with LPS in half logarithmic increasing concentrations in presence and absence of CEFTv for 19 hours at 37° C. and 5% CO₂ and the number of IFN-γ secreting cells has been detected with the ELISpot assay in a one-step and two-step manner as described in example 3.

In the one-step detection method it could be observed that LPS alone induced only in the PBMC of donor d241 and d254 as from a concentration of 10 and 100 EU/ml respectively a low secretion of IFN-γ. The number of stimulated PMC of donor d219 reached a maximum in the combination of LPS with CEFTv in a concentration of 1 EU/ml, which exhibited a high variation. Upon stimulation of the PBMC of donor d237 a constant increase of IFN-γ secreting cells as from 3.2 EU/ml could be observed. The stimulation course of the PBMC of donor d241 showed a gain of stimulated cells as from a concentration of 3.2 EU/ml which is the value of stimulated cells using LPS alone. Upon stimulation of the PBMC of donor d254 with CEFTv a lower as well as a higher number of IFN-γ secreting cells have been detected by addition of LPS to CEFTv dependent on the LPS concentration.

Using the two-step detection method an activation of IFN-γ secreting cells was observed among the PBMC of all donors upon stimulation with LPS in a concentration region of 3.2 to 32 EU/ml. Thus, the number of IFN-γ secreting cells increased analogous upon stimulation of the PBMC with CEFTv in combination with LPS. Only upon stimulation of the PBMC of donor d237 a synergy due to the combination with CEFTv could be observed at the two highest concentrations of LPS. Contrarily, upon stimulation of the PBMC of donor d254 with CEFTv combined with LPS an antagony was detected at the two highest concentrations of LPS.

The experiment showed due to the results using the two-step detection method that the stimulation ability of CEFTv could not have been improved by the addition of LPS in a stimulation preparation. Because the gain of IFN-γ secreting cells was the number of IFN-γ secreting cells activated by LPS alone. For further experiments the working concentration of LPS was set to 1 EU/ml showing no own detectable stimulating effect.

### EXAMPLE 12: ANALYSIS OF THE SYNERGISTIC EFFECT OF LPS AND CEFTV ON IFN-γ SECRETING CELLS IN PBMC

Since it could be observed that the stimulation of the PBMC with a concentration of 100 EU/ml LPS and 0.037 μg p.p./ml CEFTv resulted in a synergistic induction of IFN-γ secreting NK cells a titration of LPS with different CEFTv concentrations smaller than 1 μg p.p./ml was performed.

The PBMC used in this experiment were obtained and cultured as described in example 1.

For analysis of the influence of LPS on the activation of IFN-γ secreting cells induced by CEFTv, the PBMC adjusted to 2×10⁵ lymphocytes of 3 donors were incubated with LPS and CEFTv in various concentrations in mutual presence and absence for 19 hours at 37° C. and 5% CO₂. A matrix of stimulation preparations was formed which was composed of 8 CEFTv and 7 LPS concentrations. The number of IFN-γ secreting cells has been detected with the ELISpot assay in a one-step manner as described in example 3.

Due to the stimulation of the PBMC with CEFTv and/or LPS distinct number of cells have been stimulated to a secretion of IFN-γ. The stimulation of the PBMC of the CMV-/EBV-seropositive donor d204 induced high numbers of IFN-γ secreting cells both with CEFTv and LPS. The PBMC of CMV-seronegative/EBV-seropositive donor d237 was stimulated weakly by CEFTv. Due to the stimulation with LPS high numbers of IFN-γ secreting cells have been induced in contrast. The stimulation of the PBMC of the CMV-/EBV-seropositive donor d254 induced low numbers of IFN-γ secreting cells both with CEFTv and LPS. Also in these experiments the simultaneous administration of LPS did not result in an improvement of the stimulation ability of CEFTv in all three tested donors. In combinations of high peptide and LPS concentrations a reduced number of IFN-γ secreting cells could be observed. The threshold for LPS at which even no IFN-γ secreting cells have been detected, was confirmed at a concentration of 1 EU/ml. Thus the working concentration of LPS was remained by 1 EU/ml to examine a putative positive influence of the stimulating ability of several combined stimulants by LPS in a not own stimulating concentration.

### EXAMPLE 13: DETERMINATION OF THE COMPOSITION OF IFN-γ SECRETING LYMPHOCYTE SUBPOPULATIONS UPON STIMULATION OF THE PBMC WITH DISTINCT COMBINATIONS OF CELL-SPECIFIC STIMULANTS

To analyze if the addressed cell populations produce IFN-γ due to the stimulation of selected stimulants and how the different combinations of cell-specific stimulants affect the IFN-γ production of these cells, PBMC of a CMV/EBV-seronegative and a CMV/EBV-seropositive donor were stimulated at 37° C. and 5% CO₂. The PBMC used in this experiment were obtained and cultured as described in example 1. Since using the intracellular staining (as described in example 3) the IFN-γ production of the cells could only be measured in a time frame of 6 hours two stimulation periods have been selected of 8 and 18 hours. The stimulation time of 8 hours have been selected because here despite deviating results of the time course of stimulation with CEFTv determined by ELISA the peptide induced IFN-γ production of T helper and cytotoxic T cells was expected. Additionally a stimulation time of 18 hours was selected to have a comparable incubation period to the 19 hours stimulation period of an ELISpot assay.

The last six hours of stimulation took place in presence of BFA to prevent the secretion of IFN-γ. BFA was stained with fluorescence marked antibodies besides the CD antigens CD3, CD4, CD8 and CD56 to examine the intracellular IFN-γ production of NK, NKT-like, Th cells and CTL in the last six hours of the incubation.

The combinations tested in this experiment are shown in the table 8 below.

TABLE 8
Mixtures of different stimulants

| # | CEFTv | IL-12 | KRN7000 | LPS | Poly(I:C) | Urea |
|---|---|---|---|---|---|---|
| 1 | — | 0.01 μg/ml | 6.5 μg/ml | — | — | — |
| 2 | — | 0.01 μg/ml | — | — | 10 μg/ml | — |
| 3 | 1 μg p.p./ml | — | — | 1 EU/ml | — | — |
| 4 | 1 μg p.p./ml | — | — | — | — | 100 mM |
| 5 | 1 μg p.p./ml | — | — | 1 EU/ml | — | 100 mM |
| 6 | 1 μg p.p./ml | — | — | — | 10 μg/ml | — |
| 7 | 1 μg p.p./ml | 0.01 μg/ml | — | — | 10 μg/ml | — |
| 8 | 1 μg p.p./ml | — | — | 1 EU/ml | 10 μg/ml | — |
| 9 | 1 μg p.p./ml | 0.01 μg/ml | — | 1 EU/ml | 10 μg/ml | — |
| 10 | 1 μg p.p./ml | — | — | — | 10 μg/ml | 100 mM |
| 11 | 1 μg p.p./ml | — | 6.5 μg/ml | 1 EU/ml | 10 μg/ml | 100 mM |
| 12 | 1 μg p.p./ml | 0.01 μg/ml | 6.5 μg/ml | — | 10 μg/ml | 100 mM |
| 13 | 1 μg p.p./ml | 0.01 μg/ml | 6.5 μg/ml | 1 EU/ml | 10 μg/ml | — |
| 14 | 1 μg p.p./ml | — | 6.5 μg/ml | — | 10 μg/ml | — |
| 15 | — | 0.01 μg/ml | 6.5 μg/ml | 1 EU/ml | 10 μg/ml | 100 mM |
| 16 | 1 μg p.p./ml | 0.01 μg/ml | — | 1 EU/ml | 10 μg/ml | 100 mM |
| 17 | 1 μg p.p./ml | 0.01 μg/ml | 6.5 μg/ml | 1 EU/ml | — | 100 mM |
| 18 | 1 μg p.p./ml | 0.01 μg/ml | 6.5 μg/ml | 1 EU/ml | 10 μg/ml | 100 mM |

Abbreviations:
“—” means this compound was not added to the combination

After the stimulation of the PBMC of CMV/EBV-seropositive donor d172 for 8 hours in the period of 6 hours (2 to 8 hours) mainly IFN-γ positive Th cells were measured. In the period of 12 to 18 hours (18 hours stimulation time) also IFN-γ positive NK cells were measured which dominated the analyzed IFN-γ secreting sell subpopulations of the lymphocytes of CMV/EBV-seronegative donors. In the CMV/EBV-seropositive donor additionally IFN-γ positive cytotoxic T cells (CTL) were measured after 18 hours stimulation and similar numbers of IFN-γ positive Th and NK cells were detected dependent on the combination of the stimulants. IFN-γ positive NKT-like cells were not detected or only in very low amounts. To determine an appropriate mixture besides the results of the IFN-γ producing NK and T cells also the IFN-γ producing lymphocytes without division in subpopulations have been considered closer. For this purpose the normalized IFN-γ positive lymphocytes of both donors from both periods (2 to 8 hours and 12 to 18 hours) were added and the total result opposed in a common graphical representation.

In the selection of appropriate mixtures, it was ensured that beside a high number of IFN-γ positive cells also mixtures with comparable low donor-dependent variations are considered. Due to these criteria, the following three combinations were selected as represented in table 9:

TABLE 9
Mixtures of different stimulants

| Mixture # | CEFTv | IL-12 | LPS | Poly(I:C) | Urea |
|---|---|---|---|---|---|
| 1 | 1 μg p.p./ml | | 1 EU/ml | 10 μg/ml | — |
| 2 | 1 μg p.p./ml | 0.01 μg/ml | 1 EU/ml | 10 μg/ml | — |
| 3 | 1 μg p.p./ml | 0.01 μg/ml | 1 EU/ml | 10 μg/ml | 100 mM |

Abbreviation: “—” means this compound was not added to the combination

Since it was observed that the stimulation properties of many mixtures in CMV-seropositive donors were dominated by CEFTv and comparatively poor stimulating results in PBMC of CMV/EBV-seronegative donors were achieved, additionally mixtures without CEFTv which were not considered until then were tested. The intention was to identify a mixture of stimulants which independent of the EBV/CMV-serostatus of the donor would provide good results. Thus, the following two mixtures of table 10 were also tested.

TABLE 10
Mixtures of different stimulants

| Mixture # | CEFTv | IL-12 | LPS | Poly(I:C) | Urea |
|---|---|---|---|---|---|
| 4 | — | — | 1 EU/ml | 10 μg/ml | — |
| 5 | — | 0.01 μg/ml | 1 EU/ml | 10 μg/ml | — |

Abbreviation: “—” means this compound was not added to the combination

### EXAMPLE 14: DETERMINATION OF THE STIMULATING ACTIVITY OF VARIOUS COMBINATIONS OF CELL-SPECIFIC STIMULANTS IN A DONOR COLLECTIVE

In this experiment the PBMC of 10 donors with known CMV/EBV-serostatus were stimulated with the five different selected mixtures of example 13 and the activation of IFN-γ producing cells was analyzed using ELISpot assay as described in example 3.

The PBMC used in this experiment were obtained and cultured as described in example 1.

The mixture 3 resulted in the best stimulation results. In all preparations stimulated with mixture 3 about minimal 100 to maximal 600 IFN-γ secreting cells were detected. Due to the stimulation of the PBMC with mixtures without CEFTv (mixture 4 and 5) maximal more than 100 IFN-γ secreting cells were measured. Upon stimulations of the PBMC with mixtures containing no urea but CEFTv (mixture 1 and 2) about between 40 to 250 IFN-γ secreting cells were detected, wherein the stimulation of the PBMC of donor d034 with about 500 and of donor d270 with about 400 induced IFN-γ secreting cells formed an exception here.

Due to the results of the single stimulations, it could be observed that the stimulation of the PBMC of CMV/EBV-seropositive donors with CEFTv induced the most IFN-γ secreting cells. The exception was built by the PBMC of donor d242, which reached on all stimulations barely with IFN-γ secretion. The PBMC of CMV-seropositive/EBV-seronegative donor d268 were stimulated clearly by CEFTv. The PBMC of CMV-seronegative/EBV-seropositive donors were stimulated by CEFTv, too except the PBMC of donor d274. The stimulation of the PBMCCMV/EBV-seronegative donors did not result in significant numbers of IFN-γ secreting cells. Due to the stimulation of the PBMC with IL-12 high amounts of IFN-γ secreting cells have been detected in many donors. Due to stimulation of the PBMC with LPS in the concentration of 1 EU/ml IFN-γ secreting cells were barely detected. An exception built the PBMC of donor d258 upon its stimulation a slight activation with high variation could be observed. The PBMC of donor d270 reacted upon stimulation with LPS with a high number of IFN-γ secreting cells. Upon stimulation of the PBMC of donor d270 also upon all other stimulations high amounts of IFN-γ secreting cells could be detected with exception of urea.

Due to the stimulation of the PBMC with poly(I:C) alone between 0 and 100 IFN-γ secreting cells were detected. The exception was built again by the PMBC of donor d270 with over 270 induced IFN-γ secreting cells. The stimulation of the PBMC with urea alone did not result in a IFN-γ secretion.

Due to the stimulation of the PBMC with the mixtures 1 and 2 about 40 IFN-γ secreting cells have been detected in all three CMV/EBV-seronegative donors. The mixture 3 stimulated the PBMC of all three donors the most with a high of minimal 90 to maximal 175 IFN-γ secreting cells. Upon stimulation of the PBMC with mixture 4 and 5 the lowest results of about 10 to 20 IFN-γ secreting cells could be measured. The results showed that upon stimulation of the PBMC of donor d022 with mixture 1 to 5 a synergy was observed. Upon stimulation of the PBMC of donor d258 only with mixture 5 a synergy was detected. In donor d279 a synergy could be detected in no stimulation of the PBMC with the mixtures.

Due to the stimulation of the PBMC of the CMV-seropositive/EBV-seronegative donor with the mixtures 1 and 2 about 250 IFN-γ secreting cells have been detected. Upon stimulation of the PBMC with mixture 3 again the highest amount of about 450 IFN-γ secreting cells could be measured. Due to the mixtures 4 and 5 again the fewest stimulated cells in the region of about 40 and 60 IFN-γ secreting cells have been detected. Synergies were detectable with mixtures which did not contain IL-12 with exception of mixture 3.

The values of the stimulation of the PBMC with mixture 1 and 2 in two of three CMV-seronegative/EBV-seropositive donors lied between 160 and 250 IFN-γ secreting cells. In PBMC of donor d274 with these mixtures about 50 IFN-γ secreting cells have been induced. Upon stimulation of the PBMC with mixture 3 again the most stimulated cells have been detected. The values lied between 270 and almost 600 IFN-γ secreting cells. The fewest amounts of stimulated cells with about 50 IFN-γ secreting cells were measured upon stimulation with mixture 4 and 5. The mixture 3 stimulated the PBMC of all three donors synergistically. The mixture 1 and 2 stimulated the PBMC of donor d248 synergistically, the mixture 4 and 5 not. The PBMC of donor d253 was clearly synergistically stimulated besides mixture 3 by mixture 1. Upon stimulation with mixture 2 and 4 a synergy was detected only in the frame of the variation. The PBMC of donor d274 only reacted synergistically on mixture 3.

The stimulation of the PBMC with mixture 1 and 2 in the two of three CMV/EBV-seropositive donors d034 and d270 lied very high with 400 and 500 IFN-γ secreting cells. Upon stimulation of the PBMC of donor d242 with CEFTv only almost 40 IFN-γ secreting cells have been measured. The value of the IFN-γ secreting cells induced by mixture 3 was higher but upon stimulation of the PBMC of the donor d242 only about 95 IFN-γ secreting cells have been achieved. Upon stimulation of the PBMC of the donor d034 and d270 about 600 IFN-γ secreting cells have been detected. Upon stimulation of the PBMC with mixture 4 and 5 again in all donors the fewest numbers of IFN-γ secreting cells have been measured. Synergies were only observed upon stimulation of the PBMC of donor d034 with mixture 4 and upon stimulation of the PBMC of the donor d242 with mixture 1 and 3.

The results of the synergies are presented in the table 11.

Upon stimulation of the PBMC with mixture 3 in most of the donors a synergy could be measured in comparison to the 4 other mixtures and additionally the most IFN-γ secreting cells have been induced. Upon stimulation of the PBMC with mixture 1 and 2 in only about half of the donors a synergy was detected, with mixture 4 and 5 synergies were barely detected.

TABLE 11
Synergy of different mixtures

| | Synergy of mixtures | | | | |
|---|---|---|---|---|---|
| | | | 3 | | |
| | 1 | 2 | CEFTv + IL-12 + | | 5 |
| | CEFTv + LPS + | CEFTv + IL-12 + | LPS + Poly(I:C) + | 4 | IL-12 + LPS + |
| | Poly(I:C) | LPS + Poly(I:C) | Urea | LPS + Poly(I:C) | Poly(I:C) |
| donor | evaluation (yes/no/not clear): ΣSFC of the single components vs. SFC of the mixture ± standard deviation | | | | |
| d022 | yes: 13 vs. 36 ± 12 | Yes: 14 vs. 39 ± 13 | Yes: 14 vs. 93 ± 17 | Yes: 6 vs. 17 ± 5 | Yes: 7 vs. 19 ± 9 |
| (CMV−/EBV−) | | | | | |
| d258 | no: 58 vs. 43 ± 14 | No: 134 vs. 40 ± 8 | Yes: 135 vs. 179 ± 21 | No: 59 vs. 15 ± 4 | No: 134 vs. 16 ± 4 |
| (CMV−/EBV−) | | | | | |
| d279 | no: 74 vs. 35 ± 12 | No: 290 vs. 38 ± 14 | No: 290 vs. 124 ± 12 | No: 68 vs. 7 ± 3 | No: 283 vs. 8 ± 4 |
| (CMV−/EBV−) | | | | | |
| d268 | yes: 196 vs. 237 ± 25 | No: 362 vs. 249 ± 6 | Yes: 362 vs. 444 ± 21 | Yes: 31 vs. 63 ± 12 | No: 198 vs. 30 ± 1 |
| (CMV+/EBV+) | | | | | |
| d248 | yes: 132 vs. 226 ± 3 | Yes: 178 vs. 246 ± 37 | Yes: 180 vs. 592 ± 38 | No: 81 vs. 64 ± 4 | No: 127 vs. 79 ± 17 |
| (CMV−/EBV+) | | | | | |
| d253 | yes: 73 vs. 185 ± 29 | Not clear: 143 vs. 167 ± 27 | Yes: 143 vs. 426 ± 46 | Not clear: 25 vs. 40 ± 15 | No: 95 vs. 25 ± 7 |
| (CMV−/EBV+) | | | | | |
| d274 | no: 75 vs. 56 ± 10 | No: 255 vs. 61 ± 11 | Yes: 256 vs. 271 ± 12 | No: 73 vs. 46 ± 18 | No: 254 vs. 49 ± 6 |
| (CMV−/EBV+) | | | | | |
| d034 | Not clear: 460 vs. 476 ± 37 | No: 786 vs. 521 ± 31 | No: 786 vs. 555 ± 22 | Yes: 15 vs. 49 ± 15 | No: 341 vs. 39 ± 8 |
| (CMV+/EBV+) | | | | | |
| d242 | yes: 28 vs. 37 ± 1 | No: 41 vs. 31 ± 4 | Yes: 43 vs. 95 ± 17 | Not clear: 5 vs. 6 ± 3 | No: 17 vs. 3 ± 0 |
| (CMV+/EBV+) | | | | | |
| d270 | no: 677 vs. 393 ± 14 | No: 1094 vs. 416 ± 21 | No: 1094 vs. 597 ± 16 | No: 348 vs. 99 ± 8 | No: 765 vs. 39 ± 5 |
| (CMV+/EBV+) | | | | | |

### EXAMPLE 15: SUITABILITY OF A COCKTAIL OF PRESELECTED STIMULANTS (SUBSEQUENTLY CALLED ORIGINAL COCKTAIL) TO MEASURE THE STATUS PRIOR TO AND MONITOR ALTERATION OF CELL-MEDIATED IMMUNE RESPONSIVENESS OF RHEUMATISM PATIENTS IN THE COURSE OF TREATMENT WITH IMMUNOMODULATORY SUBSTANCES (GLUCOCORTICOIDS IN PRESENCE OR ABSENCE OF RHEUMATISM DRUGS)

The aim of this experiment was to assess the suitability of a cocktail of preselected stimulants (subsequently called original cocktail) to measure the status prior to and monitor the alteration of cell-mediated immune responsiveness in rheumatism patients in the course of treatment with glucocorticoids in presence or absence of rheumatism drugs.

The original cocktail used in these experiments includes the following components:
CEFTv peptide pool: pool of 27 peptides (composition see table 6; peptides & elephants GmbH, Potsdam, Germany) at a final working concentration per peptide of 1 μg/ml
Stock concentration per peptide: 0.37 μg/μ1 in a 1 plus 1 solution of DMSO (Sigma Aldrich, cat. no. D2650) and PBS_{LONZA} (Lonza Cologne GmbH, cat. no. BE17-516F)
IL-12 (Miltenyi, cat. no. 130-096-704) at a final working concentration of 0.01 μg/ml Stock solution: 0.01 μg/μ1 in LAL Reagent water (Lonza Cologne GmbH, cat. no. W50-100)
LPS (Sigma-Aldrich, cat. no. L4391) at a final working concentration of 1 EU/ml Stock solution: 1 mg/ml in PBS_{LONZA} (Lonza Cologne GmbH, cat. no. BE17-516F)
Poly I:C LMW ((Invivogen, cat. no. tlrt-picw) at a final working concentration of 10 μg/ml
Stock solution: 20 mg/ml in endotoxin-free water (provided by Invivogen, the manufacturer of Poly I:C))
UREA (AppliChem GmbH cat. no. A5470) at a final working concentration of 100 mM
Stock solution: 8 M urea in H₂O including 2 mM DTE, 20 mM Tris

Production of the cocktail

50 μl CEFT pool (stock solution), 18.75 μl IL-12 (stock solution), 28.75 μl from a 1:10000 dilution of the stock solution of LPS, 9.375 μl Poly I:C (stock solution) and 234 μl Urea (stock solution) were combined and filled up to a total of 500 μl with PBS_{LONZA} (Lonza Cologne GmbH, cat. no. BE17-516F).

In order to analyze the suitability of the original cocktail to assess the status prior to and to monitor functional impairment of cell-mediated immune responsiveness in rheumatism patients in the course of treatment with glucocorticoids in presence or absence with other potential immunomodulatory rheumatism drugs peripheral blood mononuclear cells (PBMC) were isolated from Li-heparinized whole blood of 20 patients with newly diagnosed rheumatic disease prior to and at indicated time points after the initiation of glucocorticoid treatment and analyzed for the presence of cocktail-responsive effector cells of cell-mediated immunity (CMI) applying a highly sensitive IFN-γ ELISpot assay (T-Track® basic IFN-γ, Lophius Biosciences GmbH, Regensburg, cat. no. 12200001). The diagnosed rheumatic diseases, basic therapy and concentrations of glucocorticoids administered to each study participant are summarized in Table 12 below.

TABLE 12
List of rheumatism patients describing the diagnosed
rheumatic disease, basic therapy and concentrations
of administered glucocorticoids.

| | | | gluco- |
|---|---|---|---|
| | | | corticoid |
| patient | diagnosis | basic therapy | (mg/day) |
| P1 | giant cell arthritis | none | 35 |
| P2 | reactive arthritis | none | 20 |
| P3 | rheumatoid arthritis | 15 mg MTX before tp3 | 20-15*¹ |
| P4 | rheumatoid arthritis | 50 mg ETN before tp3 | 20 |
| P5 | undifferentiated | 15 mg MTX before tp3 | 15 |
| | oligoarthritis | | |
| P6 | polymyalgia rheumatic | none | 15-20 |
| P7 | giant cell arteritis | none | 100-70 |
| P8 | rheumatoid arthritis | none | 15 |
| P9 | polymyalgia rheumatic | none | 20 |
| P10 | rheumatoid arthritis | 20 mg ETN before tp3 | 15-12.5 |
| P12 | rheumatoid arthritis | | 15 |
| P14 | rheumatoid arthritis | 15 mg MTX before tp3 | 12.5 |
| P15 | polymyalgia rheumatic | none | 50 |
| P16 | acute sarcoidosis | none | 50-20 |
| P17 | ANCA-associated | each 1 × 700 mg RTX | 80-60 |
| | vasculitis | before tp3, tp4 and tp5 | |
| P18 | polychondritis | none | 50-20 |
| P19 | giant cell arteritis | none | 250-80 |
| P20 | polymyalgia rheumatic | none | 15-15 |
| P21 | polymyalgia rheumatic | none | 20-25 |
| P22 | rheumatoid arthritis | none | 20 |

*¹concentrations of corticosteroids (in mg per day, daily application) administered to individual patients during the indicated observation period.
ETN: etanercep; MTX: methotrexate; P: patient; RTX: rituximab; tp: time point.

Therefore 14 μl of the original cocktail were diluted with 161 μl AIM-V medium (Life Technologies Corporation (Carlsbad, Calif., USA)) to produce the cocktail working solution. In order to analyse cell-mediated immune responsiveness of patients, each 50 μl cocktail working solution were added in quadruplicates in wells of 8 well strips pre-coated with an anti IFN-γ antibody and then filled up with 100 μl per well of the PBMC solution in a concentration of PBMC adjusted to 2×10⁵ lymphocytes per well. For negative control, 50 μl AIM-V medium (Gibco, cat. no. 31035-025) were added in quadruplicates in wells of the 8 well strips pre-coated with an anti IFN-γ antibody and filled up with 100 μl per well of the PBMC solution in a concentration of PBMC adjusted to 2×10⁵ lymphocytes per well. Last, a positive control well included 50 μl of a 2 μg/ml SEB solution (Enterotoxin B from Staphylococcus aureus (Sigma-Aldrich Co. LLC., St. Luis, Mo., USA, cat. no. S881 filled up with 100 μl of the PBMC solution per well in a concentration of PBMC adjusted to 2×10⁵ lymphocytes per well. Then, stripes (laced in frames) were incubated for 19 hours at 37° C. and 5% CO₂. After removal of cell suspension stripes were washed six times for each 3 minutes with 200 μl 0.01% Tween20 (Merck KGaA, Darmstadt) per well. Then, 100 μl detection conjugate MAK<h-IFNg>M-7-B6-10-AP(Ox) (MicroCoat GmbH, Bernried, Germany) were added in a concentration of 0.4 U/ml (diluted in detection conjugate buffer 1 (MicroCoat GmbH, Bernried)) per well and incubated for two hours at 37° C. and 5% CO₂. Then wells was washed three times for each 3 minutes with 200 μl 0.01 Tween20 (Merck KGaA, Darmstadt) (diluted in PBSo (LONZA, Verviers, Belgium, cat. no. BE17-516F)) per well and then washed three times each 3 minutes with 200 μl PBSo (LONZA). Finally, staining of spots was performed by incubating wells for 6 minutes in the dark with 50 μl of a 1-step nitroblue tetrazolium-5-bromo-4-chloro-3-indolylphosphate (NBT/BCIP) substrate (Thermo Fischer Scientific, Waltham, USA). The staining reaction was stopped by rinsing the plates three times with water. The stripes were then striked on an adsorbent paper, the bottom plate was removed and the membrane was carefully pressed on the tissue. The analysis of the plates was performed using the BioReader 5000-Ea (Bio-Sys GmbH Karben) after drying of the plates overnight. Isolation, stimulation and culture of cells were performed under sterile conditions.

A significant number of responsive cells before the initiation of treatment with immunomodulatory substances—defined as more than 10 spot forming cells (SFC)/2×10⁵ PBMC and a number of SFC 2.5 times higher in the cocktail stimulated cells compared to the unstimulated control cells—could be observed in 17 of 20 patients (85%) prior to the start of treatment allowing for monitoring of CMI in the majority of patients. Treatment of rheumatism patients with glucocorticoids and/or other immunomodulatory rheumatism drugs resulted in an individual impairment of cell-mediated immune responsiveness, as a significant decrease between 13 to 63% of IFN-γ secreting cells could be observed in patients with a daily glucocorticoid dosage of at least 35 mg prednisolone equivalent (n=6, 30%), whereas no or only a minimal decrease (13%) of IFN-γ secreting cells was detectable in some of the patients (n=2, 10%) treated with the highest glucocorticoid dosages (100 or 80 mg prednisolone equivalent/day (p)).

The present results demonstrate substantial differences in the frequency of functional cells in patients suffering from rheumatic diseases following initiation of immunosuppression. We propose that significant drop in the numbers of cocktail-reactive cells reflects an incremental deterioration of the immune network putting the patient to a higher risk for infection.

Substantial increase in the numbers of cocktail-reactive cells may indicate ongoing activation and expansion of pathogen-reactive effector cells of CMI in response to pathogen-replication. Therefore, the utilization of the novel test has the potential to guide immunosuppression as a valuable tool for functional immune monitoring.

Exemplarily, patient P1 suffering from a newly diagnosed giant cell arthritis and thus treated daily with 35 mg prednisolone equivalent without any standard therapy showed continuous drop in the numbers of cocktail-reactive IFN-g-producing cells at days 6 and 9 post initiation of treatment with prednisolone (FIG. 17 A), whereas patient P3 (with newly diagnosed rheumatoid arthritis) receiving a daily dose between 20 and 15 mg prednisolone equivalent in addition to a basic therapy of 15 mg methotrexate at day 5 post initiation of treatment with glycocorticoid showed substantially increased numbers of cocktail-reactive IFN-γ producing cells. This patient showed a weak CMV reactivation within the observation time of day 0 to 9 post initiation of glucocorticoid treatment, which was self-clearing without antiviral therapy (FIG. 17 B).

### EXAMPLE 16: COMPARATIVE ANALYSIS OF THE SUITABILITY OF THE ORIGINAL COCKTAIL AND DIFFERENT CMV ANTIGENS TO ASSESS AND MONITOR CELL-MEDIATED IMMUNE RESPONSIVENESS IN LYMPHOCYTES OF A RHEUMATIC PATIENT PRIOR TO AND AT DIFFERENT TIME POINTS IN THE COURSE OF IMMUNOSUPPRESSIVE TREATMENT

The aim of this experiment was to compare the suitability of the original cocktail and different cytomegalovirus (CMV) antigens to assess cell-mediated immune responsiveness in freshly isolated lymphocytes of a CMV seropositive rheumatism patient prior to and in the course of treatment with immunomodulatory substances (glucocorticoids in presence or absence of rheumatism drugs). Tested CMV-specific stimulants included T-activated CMV IE-1 and pp65 antigens (Lophius Biosciences GmbH, Regensburg, cat. no. 12311001 and 12311002) as well as an IE-1 Maxipool (pool of 120 15-mer peptides with 11 amino acids (aa) overlap covering the complete CMV IE-1 protein; Towne strain) and a pp65 Maxipool (pool of 44 15-mer peptides with 11 aa overlap covering aa 366-546 of the CMV pp65 protein; strain AD169)) (peptides & elephants GmbH, Potsdam, Germany).

The CMV-seropositive rheumatic patient 4 (P4) has been newly diagnosed to be suffering from rheumatoid arthritis and was treated within the observation period of day 0 to 6 post start of glucocorticoid treatment with 20 mg prednisolone equivalent/day. At day 5 the patient received basic therapy of 50 mg ETN.

In order to compare suitability of the original cocktail and different cytomegalovirus (CMV) antigens to assess and monitor cell-mediated immune responsiveness in lymphocytes of rheumatic patient P4 prior to and at different time points in the course of immunosuppressive treatment, 50 μl working solution of the original cocktail (1) or 50 μl AIM-V medium including either 3 μg/ml T-activated pp65 (2), 1 μg/ml/peptide pp65 Maxipool (pool of 44 15-mer peptides with 11 aa overlap covering aa 366-546 of the CMV pp65 protein; strain AD169) (3)), 3 μg/ml/peptide IE-1 Maxipool (pool of 120 15-mer peptides with 11 aa overlap covering the complete CMV IE-1 protein; Towne strain) (4)) or 15 μg/ml T-activated IE-1 (5) were added in quadruplicates in wells of 8 well strips pre-coated with an anti IFN-γ antibody and then filled up with 100 μl per well of the PBMC solution in a concentration of PBMC adjusted to 2×10⁵ lymphocytes per well. For negative control, 50 μl AIM-V medium (Gibco, cat. no. 31035-025) were added in quadruplicates in wells of the 8 well strips pre-coated with an anti IFN-γ antibody and filled up with 100 μl per well of the PBMC solution in a concentration of PBMC adjusted to 2×10⁵ lymphocytes per well. Incubation of cells as well as IFN-γ ELISpot assay were performed as essentially described in example 15.

In this experiment, stimulation of lymphocytes obtained prior to the start of treatment with glucocorticoids (d0) with the original cocktail resulted in the activation of substantional numbers of cells (app. 550/2×10⁵ cells) for the production of IFN-γ. In this patient a slight increase of original cocktail-reactive cells was observed within the observation period of six days post start of glucocorticoid treatment resulting in 615 and app. 600 spots at days 2 and 6. Herein, stimulation of cells with all other tested CMV antigens resulted in substantially reduced numbers of reactive, IFN-g producing cells with maximum spot counts of app. 450 for T activated pp65 at day 2, 140 spots for T activated IE-1 at day 6, app. 350 spots for the pp65 Maxipool at day 2, app. 270 spots for the IE-1 Maxipool at day 6.

Notably, spot counts of all tested stimulants showed a similar course within the observational period of 6 days after the initiation of glucocorticoid treatment.

This experiment shows, that even in CMV-seropositive patients with high numbers of CMV-reactive T cells, the original cocktail was more efficient in the monitoring of cell-mediated immune responsiveness than well established and commonly used CMV-specific stimulants.

### EXAMPLE 17: COMPARATIVE ANALYSIS OF THE SUITABILITY OF THE ORIGINAL COCKTAIL INCLUDING THE POOL OF CEFTV PEPTIDES AND A MODIFIED COCKTAIL INCLUDING DYNABEADS® HUMAN T-ACTIVATOR CD3/CD28 INSTEAD OF THE POOL OF CEFTV PEPTIDES TO ASSESS AND MONITOR CELL-MEDIATED IMMUNE RESPONSIVENESS IN PBMC OF THREE HEALTHY DONORS D22, D295, D296)

The aim of this experiment was to compare the effectiveness of the original cocktail which includes the pool of CEFTv peptides for an antigen-specific stimulation of T cells with a modified cocktail including Dynabeads® Human T-Activator CD3/CD28 instead of the CEFTv pool as a component for the unspecific activation of T cells.

For the production of modified cocktail 5 μl of Dynabeads® Human T-Activator CD3/CD28 solution (ThermoFisher, cat. no. 111.61D) (suitable for the stimulation of 200.000 cells) were washed 3 times with PBS. After the last washing step, the beads were resuspended in 4 μl Cocktail w/o CEFTv or 4 μl PBS, respectively.

In order to compare effectiveness of the original cocktail with the modified cocktail 50 μl working solution of the original cocktail, 50 μl working solution of the original cocktail without the pool of CEFTv peptides, 50 μl of AIM-V medium including Human T-Activator CD3/CD28 beads for 2×10⁵ cells or 50 μl working solution of the cocktail without the pool of CEFTv peptides but including Human T-Activator CD3/CD28 recommended for 2×10⁵ cells were added in quadruplicates in wells of 8 well strips pre-coated with an anti IFN-γ antibody and then filled up with 100 μl per well of AIM-V medium including 2×10⁵ PBMC. Incubation of cells as well as IFN-γ ELISpot assay were performed as essentially described in example 15.

In all three experiments modified cocktail including Dynabeads® Human T-Activator CD3/CD28 instead of the CEFTv pool showed substantially higher stimulatory capacity when compared to the original cocktail or cocktail without CEFTv. Notably, in PBMC of donor d022, modified cocktail including Dynabeads® Human T-Activator CD3/CD28 instead of the CEFTv pool showed a stronger stimulatory capacity than the Dynabeads® Human T-Activator CD3/CD28 instead of the CEFTv pool. In PBMC of d295 and d296, spot counts upon stimulation with modified cocktail including Dynabeads® Human T-Activator CD3/CD28 instead of the CEFTv pool and the Dynabeads® Human T-Activator CD3/CD28 were >1000 and thus are not directly comparable.

1. A composition comprising:
i) a first substance which is capable to stimulate T cells,
ii) a second substance which is capable to stimulate NK cells (natural killer cells), and
iii) lipopolysaccharide (LPS) and urea,
wherein the first substance is a peptide pool, a protein, a peptide, and/or an antibody and/or wherein the second substance is a double stranded nucleic acid, single stranded nucleic acid, unmethylated CpG oligodeoxynucleotide, TLR agonist except lipopolysaccharide (LPS), arabinoxylan (BioBran® MGN-3), an immunoglobulin, a murine cytomegalovirus (MCMV)-encoded protein, CCL5 (chemokine (C—C motif) ligand 5), a UL-16-binding protein (ULBP), CD48, CD70, CD155, CD112, Necl-1, B7-H6, ICAM-1, RAE-1 (retinoic acid early inducible 1), H60, Multi and/or hemagglutinin.

2. The composition according to claim 1, wherein the composition additionally comprises an enhancer.

3. The composition according to claim 1, wherein the first substance is (i) a peptide pool comprising at least two peptides which are each a complete protein antigen, or a part thereof, derived from distinct species, wherein the peptides have a length ranging from 18 to 31 amino acids and/or (ii) an anti-CD3 antibody, TGN1412, anti-CD28 antibody and/or anti-CD49 antibody and/or any combination thereof, and/or (iv) a stimulant or (v) pp65 and/or a fragment thereof.

4. The composition according to claim 3, wherein the murine cytomegalovirus-encoded protein is m157, the ULBP is ULBP1, ULBP2, ULBP3, ULBP4, ULBP5 or ULBP6, the immunoglobulin is IgG, the hemagglutinin is a viral hemagglutinin, the TLR (Toll-like receptor) agonist is imidazoquinoline, R848, lipomannan, polyinosinic:polycytidylic acid (poly(I:C)), poly(I:C)-LMW (low molecular weight), poly(I:C)-LMW/LyoVec, Pam3CS 4 and CpG oligodeoxynucleotides.

5. The composition according to claim 1, wherein the composition comprises LPS and/or urea each in a concentration which is not capable to stimulate immune cells to release an immune effector molecule if LPS or urea is applied alone.

6. The composition according to claim 1, wherein the concentration of urea is 10, 20, 30, 40, 50, 100, 150, 200, 300, 400, 500, 600, 700, 800, 900 or 1000 mM and/or the concentration of LPS is 0.5, 0.75, 1, 1.5, 2, 2.5, 3, 4, 5, 6, 7, 8, 9, 10, 11, 12, 13, 14, 15, 16, 15, 16, 17, 18, 19 or 20 EU/ml.

7. A method for measuring, determining and/or detecting the status of cell-mediated immune responsiveness of a subject, the method comprising:
a) contacting a sample from the subject with the composition according to any one of claim 1, and
b) detecting the presence or elevation in the level of at least one immune effector molecule from immune cells, wherein the presence or level of the immune effector molecule is indicative of the level of cell-mediated responsiveness of the subject.

8. The method according to claim 7, wherein the method further comprises the step c) comparing the detected immune effector molecule level with a reference-level.

9. A kit comprising the composition according to claim 1.

10. The kit according to claim 9, wherein the single components of the composition are provided in separate vials or tubes.

11. (canceled)

12. The method of claim 7, wherein said subject has or is suspected of having an immunosuppression condition.

13. A peptide pool comprising at least two peptides which are each a complete protein antigen, or a part thereof, derived from distinct species, wherein the peptides have a length ranging from 18 to 31 amino acids.

14. The peptide pool according to claim 13, wherein the peptide pool comprises 20, 21, 22, 23, 24, 25, 26, 27, 28, 29 30, 31, 32, 34, 35, 35, 36, 37, 38, 39 or 40 peptides.

15. The peptide pool according to claim 13, wherein the at least two peptides are each a complete protein antigen, or a part thereof, derived from the influenza virus, Epstein-Barr virus, cytomegalovirus and Clostridium tetani.

16. The peptide pool according to claim 13, wherein the peptide pool is a CEFT pool comprising peptides that are extended at their respective N- and C-terminus by at least one, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 31, 32, 34, 35, 36, 37, 38, 39, 40, 50, 60, 70, 80, 90, 100 or more amino acids.

17. The peptide pool according to claim 13, wherein said extension reflects the wild type amino acid sequence that is found at the N- and C-terminal end of the respective peptide when compared to the antigen from which this peptide was derived from.

18. The peptide pool according to claim 13, wherein the peptide pool comprises the peptides according to SEQ ID NO: 1 to 27.

19. The composition of claim 3, wherein the least two peptides are derived from two or more distinct species, and/or wherein the stimulant is selected from the group consisting of Staphylococcal enterotoxin B (SEB), plant lectin, such as phytohemagglutinin (PHA) and pokeweed mitogen (PWM).

20. The composition of claim 4, wherein the viral hemagglutinin is an influenza hemagglutinin.

21. The peptide pool of claim 13, wherein the least two peptides are derived from two or more distinct species.


SEQUENCE LISTING (88 sequences)

SEQ ID NO: 1 - protein, 19 aa, Influenza virus
KAGLLVSDGGPNLYNIRNL

SEQ ID NO: 2 - protein, 19 aa, Influenza virus
FYIQMCTELKLSDYEGRLI

SEQ ID NO: 3 - protein, 19 aa, Influenza virus
SPLTKGILGFVFTLTVPSE

SEQ ID NO: 4 - protein, 19 aa, Epstein Barr virus
AIQNAGLCTLVAMLEETIF

SEQ ID NO: 5 - protein, 19 aa, Cytomegalovirus
GILARNLVPMVATVQGQNL

SEQ ID NO: 6 - protein, 19 aa, Influenza virus
GKDPKKTGGPIYKRVDRKW

SEQ ID NO: 7 - protein, 19 aa, Influenza virus
ARSALILRGSVAHKSCLPA

SEQ ID NO: 8 - protein, 19 aa, Epstein Barr virus
LLKHSRVRAYTYSKVLGVD

SEQ ID NO: 9 - protein, 19 aa, Epstein Barr virus
RDRLARLRAEAQVKQASVE

SEQ ID NO: 10 - protein, 19 aa, Epstein Barr virus
KKCRAIVTDFSVIKAIEEE

SEQ ID NO: 11 - protein, 19 aa, Epstein Barr Virus
RVMIPATIGTAMYKLLKHS

SEQ ID NO: 12 - protein, 20 aa, Epstein Barr virus
GSLVSDYCNVLNKEFTAGSV

SEQ ID NO: 13 - protein, 19 aa, Epstein Barr virus
GPKVKRPPIFIRRLHRLLL

SEQ ID NO: 14 - protein, 19 aa, Influenza virus
DSNTLELRSRYWAIRTRSG

SEQ ID NO: 15 - protein, 18 aa, Epstein Barr virus
AARKSRAKFKQLLQHYRE

SEQ ID NO: 16 - protein, 19 aa, Epstein Barr virus
AWNAGFLRGRAYGLDLLRT

SEQ ID NO: 17 - protein, 19 aa, Epstein Barr virus
ASRRDQAKWRLQTLAAGWP

SEQ ID NO: 18 - protein, 19 aa, Influenza virus
TLELRSRYWAIRTRSGGNT

SEQ ID NO: 19 - protein, 19 aa, Epstein Barr virus
WQRRYRRIYDLIELCGSLH

SEQ ID NO: 20 - protein, 19 aa, Epstein Barr virus
AQGMAYPLHEQHGMAPCPV

SEQ ID NO: 21 - protein, 20 aa, Epstein Barr virus
NLLQTEENLLDFVRFMGVMS

SEQ ID NO: 22 - protein, 20 aa, Cytomegalovirus
NLKYQEFFWDANDIYRIFAE

SEQ ID NO: 23 - protein, 20 aa, Cytomegalovirus
TTERKTPRVTGGGAMAGAST

SEQ ID NO: 24 - protein, 25 aa, Clostridium tetani
KNILMQYIKANSKFIGITELKKLES

SEQ ID NO: 25 - protein, 31 aa, Clostridium tetani
EYNDMFNNFTVSFWLRVPKVSASHLEQYGTN

SEQ ID NO: 26 - protein, 26 aa, Clostridium tetani
LYNGLKFIIKRYTPNNEIDSFVKSGD

SEQ ID NO: 27 - protein, 26 aa, Clostridium tetani
NNNQYVSIDKFRIFCKALNPKEIEKL

SEQ ID NO: 28 - protein, 9 aa, Influenza A virus
VSDGGPNLY

SEQ ID NO: 29 - protein, 9 aa, Influenza A virus
CTELKLSDY

SEQ ID NO: 30 - protein, 9 aa, Influenza A virus
GILGFVFTL

SEQ ID NO: 31 - protein, 9 aa, Influenza A virus
FMYSDFHFI

SEQ ID NO: 32 - protein, 9 aa, Epstein Barr virus
CLGGLLTMV

SEQ ID NO: 33 - protein, 9 aa, Epstein Barr virus
GLCTLVAML

SEQ ID NO: 34 - protein, 9 aa, Cytomegalovirus
NLVPMVATV

SEQ ID NO: 35 - protein, 10 aa, Influenza A virus
RVLSFIKGTK

SEQ ID NO: 36 - protein, 9 aa, Influenza A virus
ILRGSVAHK

SEQ ID NO: 37 - protein, 9 aa, Epstein Barr virus
RVRAYTYSK

SEQ ID NO: 38 - protein, 9 aa, Epstein Barr virus
RLRAEAQVK

SEQ ID NO: 39 - protein, 9 aa, Influenza A virus
SIIPSGPLK

SEQ ID NO: 40 - protein, 10 aa, Epstein Barr virus
AVFDRKSDAK

SEQ ID NO: 41 - protein, 9 aa, Epstein Barr virus
IVTDFSVIK

SEQ ID NO: 42 - protein, 9 aa, Epstein Barr virus
ATIGTAMYK

SEQ ID NO: 43 - protein, 10 aa, Epstein Barr virus
DYCNVLNKEF

SEQ ID NO: 44 - protein, 9 aa, Influenza A virus
KTGGPIYKR

SEQ ID NO: 45 - protein, 9 aa, Influenza A virus
LPFDKTTVM

SEQ ID NO: 46 - protein, 9 aa, Epstein Barr virus
RPPIFIRRL

SEQ ID NO: 47 - protein, 10 aa, Cytomegalovirus
TPRVTGGGAM

SEQ ID NO: 48 - protein, 9 aa, Influenza A virus
ELRSRYWAI

SEQ ID NO: 49 - protein, 8 aa, Epstein Barr virus
RAKFKQLL

SEQ ID NO: 50 - protein, 9 aa, Epstein Barr virus
FLRGRAYGL

SEQ ID NO: 51 - protein, 9 aa, Epstein Barr virus
QAKWRLQTL

SEQ ID NO: 52 - protein, 10 aa, Cytomegalovirus
EFFWDANDIY

SEQ ID NO: 53 - protein, 12 aa, Cytomegalovirus
SDEEEAIVAYTL

SEQ ID NO: 54 - protein, 9 aa, Influenza A virus
SRYWAIRTR

SEQ ID NO: 55 - protein, 8 aa, Influenza A virus
ASCMGLIY

SEQ ID NO: 56 - protein, 9 aa, Epstein Barr virus
RRIYDLIEL

SEQ ID NO: 57 - protein, 9 aa, Epstein Barr virus
YPLHEQHGM

SEQ ID NO: 58 - protein, 9 aa, Cytomegalovirus
IPSINVHHY

SEQ ID NO: 59 - protein, 10 aa, Epstein Barr virus
EENLLDFVRF

SEQ ID NO: 60 - protein, 15 aa, Clostridium tetani
QYIKANSKFIGITEL

SEQ ID NO: 61 - protein, 21 aa, Clostridium tetani
FNNFTVSFWLRVPKVSASHLE

SEQ ID NO: 62 - protein, 16 aa, Clostridium tetani
KFIIKRYTPNNEIDSF

SEQ ID NO: 63 - protein, 16 aa, Clostridium tetani
VSIDKFRIFCKALNPK

SEQ ID NO: 64 - protein, 11 aa, Influenza A virus
FVFTLTVPSER

SEQ ID NO: 65 - protein, 15 aa, Influenza A virus
SGPLKAEIAQRLEDV

SEQ ID NO: 66 - protein, 16 aa, Influenza A virus
YDVPDYASLRSLVASS

SEQ ID NO: 67 - protein, 13 aa, Influenza B virus
PYYTGEHAKAIGN

SEQ ID NO: 68 - protein, 12 aa, Clostridium tetani
GQIGNDPNRDIL

SEQ ID NO: 69 - protein, 12 aa, Influenza A virus
PKYVKQNTLKLA

SEQ ID NO: 70 - protein, 13 aa, Influenza A virus
PKYVKQNTLKLAT

SEQ ID NO: 71 - protein, 9 aa, Influenza A virus
DRLRRDQKS

SEQ ID NO: 72 - protein, 19 aa, Epstein Barr virus
AGLTLSLLVICSYLFISRG

SEQ ID NO: 73 - protein, 14 aa, Clostridium tetani
QYIKANSKFIGITE

SEQ ID NO: 74 - protein, 13 aa, Epstein Barr virus
TSLYNLRRGRALA

SEQ ID NO: 75 - protein, 18 aa, Epstein Barr virus
VPGLYSPCRAFFNKEELL

SEQ ID NO: 76 - protein, 14 aa, Cytomegalovirus
DKREMWMACIKELH

SEQ ID NO: 77 - protein, 15 aa, Epstein Barr virus
TGHGARTSTEPTTDY

SEQ ID NO: 78 - protein, 13 aa, Epstein Barr virus
KELKRQYEKKLRQ

SEQ ID NO: 79 - protein, 16 aa, Influenza A virus
RGYFKMRTGKSSIMRS

SEQ ID NO: 80 - protein, 11 aa, Epstein Barr virus
TVFYNIPPMPL

SEQ ID NO: 81 - protein, 15 aa, Epstein Barr virus
AEGLRALLARSHVER

SEQ ID NO: 82 - protein, 20 aa, Epstein Barr virus
PGPLRESIVCYFMVFLQTHI

SEQ ID NO: 83 - protein, 561 aa, Cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 84 - protein, 186 aa, Cytomegalovirus
MRIRMHPTFTSQYRIQGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDA

SEQ ID NO: 85 - protein, 187 aa, Cytomegalovirus
MRIRMHPTFTSQYRIQGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDAL

SEQ ID NO: 86 - protein, 182 aa, Cytomegalovirus
HPTFTSQYRIQGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDAL

SEQ ID NO: 87 - protein, 194 aa, artificial sequence
  CMV pp65 fragment with HIS-tag
MRIRMHPTFTSQYRIQGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALEHHHHHH

SEQ ID NO: 88 - protein, 29 aa, Cytomegalovirus
PAAQPKRRRHRQDALPGPCIASTPKKHRG